SEQUENCE LISTING (41 sequences)

SEQ ID NO: 1 - protein, 1061 aa, Homo sapiens
MPGPRRPAGSRLRLLLLLLLPPLLLLLRGSHAGNLTVAVVLPLANTSYPWSWARVGPAVELALAQVKARPDLLPGWTVRTVLGSSENALGVCSDTAAPLAAVDLKWEHNPAVFLGPGCVYAAAPVGRFTAHWRVPLLTAGAPALGFGVKDEYALTTRAGPSYAKLGDFVAALHRRLGWERQALMLYAYRPGDEEHCFFLVEGLFMRVRDRLNITVDHLEFAEDDLSHYTRLLRTMPRKGRVIYICSSPDAFRTLMLLALEAGLCGEDYVFFHLDIFGQSLQGGQGPAPRRPWERGDGQDVSARQAFQAAKIITYKDPDNPEYLEFLKQLKHLAYEQFNFTMEDGLVNTIPASFHDGLLLYIQAVTETLAHGGTVTDGENITQRMWNRSFQGVTGYLKIDSSGDRETDFSLWDMDPENGAFRVVLNYNGTSQELVAVSGRKLNWPLGYPPPDIPKCGFDNEDPACNQDHLSTLEVLALVGSLSLLGILIVSFFIYRKMQLEKELASELWRVRWEDVEPSSLERHLRSAGSRLTLSGRGSNYGSLLTTEGQFQVFAKTAYYKGNLVAVKRVNRKRIELTRKVLFELKHMRDVQNEHLTRFVGACTDPPNICILTEYCPRGSLQDILENESITLDWMFRYSLTNDIVKGMLFLHNGAICSHGNLKSSNCVVDGRFVLKITDYGLESFRDLDPEQGHTVYAKKLWTAPELLRMASPPVRGSQAGDVYSFGIILQEIALRSGVFHVEGLDLSPKEIIERVTRGEQPPFRPSLALQSHLEELGLLMQRCWAEDPQERPPFQQIRLTLRKFNRENSSNILDNLLSRMEQYANNLEELVEERTQAYLEEKRKAEALLYQILPHSVAEQLKRGETVQAEAFDSVTIYFSDIVGFTALSAESTPMQVVTLLNDLYTCFDAVIDNFDVYKVETIGDAYMVVSGLPVRNGRLHACEVARMALALLDAVRSFRIRHRPQEQLRLRIGIHTGPVCAGVVGLKMPRYCLFGDTVNTASRMESNGEALKIHLSSETKAVLEEFGGFELELRGDVEMKGKGKVRTYWLLGERGSSTRG

SEQ ID NO: 2 - protein, 1057 aa, Mus musculus
MPGSRRVRPRLRALLLLPPLLLLRSGHASDLTVAVVLPLTNTSYPWSWARVGPAVELALGRVKARPDLLPGWTVRMVLGSSENAAGVCSDTAAPLAAVDLKWEHSPAVFLGPGCVYSAAPVGRFTAHWRVPLLTAGAPALGIGVKDEYALTTRTGPSHVKLGDFVTALHRRLGWEHQALVLYADRLGDDRPCFFIVEGLYMRVRERLNITVNHQEFVEGDPDHYTKLLRTVQRKGRVIYICSSPDAFRNLMLLALDAGLTGEDYVFFHLDVFGQSLQGAQGPVPRKPWERDDGQDRRARQAFQAAKIITYKEPDNPEYLEFLKQLKLLADKKFNFTMEDGLKNIIPASFHDGLLLYVQAVTETLAQGGTVTDGENITQRMWNRSFQGVTGYLKIDRNGDRDTDFSLWDMDPETGAFRVVLNFNGTSQELMAVSEHRLYWPLGYPPPDIPKCGFDNEDPACNQDHFSTLEVLALVGSLSLVSFLIVSFFIYRKMQLEKELVSELWRVRWEDLQPSSLERHLRSAGSRLTLSGRGSNYGSLLTTEGQFQVFAKTAYYKGNLVAVKRVNRKRIELTRKVLFELKHMRDVQNEHLTRFVGACTDPPNICILTEYCPRGSLQDILENESITLDWMFRYSLTNDIVKGMLFLHNGAIGSHGNLKSSNCVVDGRFVLKITDYGLESFRDPEPEQGHTLFAKKLWTAPELLRMASPPARGSQAGDVYSFGIILQEIALRSGVFYVEGLDLSPKEIIERVTRGEQPPFRPSMDLQSHLEELGQLMQRCWAEDPQERPPFQQIRLALRKFNKENSSNILDNLLSRMEQYANNLEELVEERTQAYLEEKRKAEALLYQILPHSVAEQLKRGETVQAEAFDSVTIYFSDIVGFTALSAESTPMQVVTLLNDLYTCFDAVIDNFDVYKVETIGDAYMVVSGLPVRNGQLHAREVARMALALLDAVRSFRIRHRPQEQLRLRIGIHTGPVCAGVVGLKMPRYCLFGDTVNTASRMESNGEALRIHLSSETKAVLEEFDGFELELRGDVEMKGKGKVRTYWLLGERGCSTRG

SEQ ID NO: 3 - protein, 1057 aa, Rattus norvegicus
MPGSRRVRPRLRALLLLPPLLLLRGGHASDLTVAVVLPLTNTSYPWSWARVGPAVELALARVKARPDLLPGWTVRMVLGSSENAAGVCSDTAAPLAAVDLKWEHSPAVFLGPGCVYSAAPVGRFTAHWRVPLLTAGAPALGIGVKDEYALTTRTGPSHVKLGDFVTALHRRLGWEHQALVLYADRLGDDRPCFFIVEGLYMRVRERLNITVNHQEFVEGDPDHYPKLLRAVRRKGRVIYICSSPDAFRNLMLLALNAGLTGEDYVFFHLDVFGQSLKSAQGLVPQKPWERGDGQDRSARQAFQAAKIITYKEPDNPEYLEFLKQLKLLADKKFNFTVEDGLKNIIPASFHDGLLLYVQAVTETLAQGGTVTDGENITQRMWNRSFQGVTGYLKIDRNGDRDTDFSLWDMDPETGAFRVVLNYNGTSQELMAVSEHKLYWPLGYPPPDVPKCGFDNEDPACNQDHFSTLEVLALVGSLSLISFLIVSFFIYRKMQLEKELVSELWRVRWEDLQPSSLERHLRSAGSRLTLSGRGSNYGSLLTTEGQFQVFAKTAYYKGNLVAVKRVNRKRIELTRKVLFELKHMRDVQNEHLTRFVGACTDPPNICILTEYCPRGSLQDILENESITLDWMFRYSLTNDIVKGMLFLHNGAICSHGNLKSSNCVVDGRFVLKITDYGLESFRDPEPEQGHTLFAKKLWTAPELLRMASPPARGSQAGDVYSFGIILQEIALRSGVFYVEGLDLSPKEIIERVTRGEQPPFRPSMDLQSHLEELGQLMQRCWAEDPQERPPFQQIRLALRKFNKENSSNILDNLLSRMEQYANNLEELVEERTQAYLEEKRKAEALLYQILPHSVAEQLKRGETVQAEAFDSVTIYFSDIVGFTALSAESTPMQVVTLLNDLYTCFDAVIDNFDVYKVETIGDAYMVVSGLPVRNGQLHAREVARMALALLDAVRSFRIRHRPQEQLRLRIGIHTGPVCAGVVGLKMPRYCLFGDTVNTASRMESNGEALKIHLSSETKAVLEEFDGFELELRGDVEMKGKGKVRTYWLLGERGCSTRG

SEQ ID NO: 4 - protein, 10 aa, Homo sapiens
GFTFSSYWMN

SEQ ID NO: 5 - protein, 17 aa, Homo sapiens
VIESKGNYIFYADSVKG

SEQ ID NO: 6 - protein, 16 aa, Homo sapiens
DRYSMIYSYGAGAFDY

SEQ ID NO: 7 - protein, 5 aa, Homo sapiens
SYWMN

SEQ ID NO: 8 - protein, 7 aa, Homo sapiens
GFTFSSY

SEQ ID NO: 9 - protein, 6 aa, Homo sapiens
ESKGNY

SEQ ID NO: 10 - protein, 8 aa, Homo sapiens
GFTFSSYW

SEQ ID NO: 11 - protein, 8 aa, Homo sapiens
IESKGNYI

SEQ ID NO: 12 - protein, 18 aa, Homo sapiens
ARDRYSMIYSYGAGAFDY

SEQ ID NO: 13 - protein, 125 aa, Homo sapiens
EVQLLESGGGLVQPGGSLRLSCAASGFTFSSYWMNWVRQAPGKGLEWVSVIESKGNYIFYADSVKGRFTISRDNSKNTLYLQMNSLRAEDTAVYYCARDRYSMIYSYGAGAFDYWGQGTLVTVSS

SEQ ID NO: 14 - DNA, 375 bp, Homo sapiens
  source 1..375: mol_type = other DNA
gaagtgcagctgctggaatccggcggaggtctggtccagcctggaggttccctgcgcctgtcatgcgcagcctccggattcaccttttcgtcgtactggatgaactgggtcagacaggctcctggaaagggcctggaatgggtgtctgtgattgaatccaaggggaactacatcttctacgcggacagcgtgaagggccggttcactatcagcagagacaacagcaagaacaccctgtacctccaaatgaactcgctgagggccgaagatactgccgtgtactactgtgcccgcgatcgctactcgatgatctacagctatggtgccggagcgttcgattactggggacagggaaccctcgtgaccgtcagctcc

SEQ ID NO: 15 - protein, 455 aa, Homo sapiens
EVQLLESGGGLVQPGGSLRLSCAASGFTFSSYWMNWVRQAPGKGLEWVSVIESKGNYIFYADSVKGRFTISRDNSKNTLYLQMNSLRAEDTAVYYCARDRYSMIYSYGAGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - DNA, 1365 bp, Homo sapiens
  source 1..1365: mol_type = other DNA
gaagtgcagctgctggaatccggcggaggtctggtccagcctggaggttccctgcgcctgtcatgcgcagcctccggattcaccttttcgtcgtactggatgaactgggtcagacaggctcctggaaagggcctggaatgggtgtctgtgattgaatccaaggggaactacatcttctacgcggacagcgtgaagggccggttcactatcagcagagacaacagcaagaacaccctgtacctccaaatgaactcgctgagggccgaagatactgccgtgtactactgtgcccgcgatcgctactcgatgatctacagctatggtgccggagcgttcgattactggggacagggaaccctcgtgaccgtcagctccgcctcaaccaagggcccgtcagtgttcccgctggctccatcgtcgaagtccacctccggaggaaccgcagcactcggttgcctggtcaaggactacttccctgagccagtgaccgtgtcgtggaacagcggagccctgacttccggcgtgcacacttttcccgcggtgctgcagtcctccggtctgtactccctttcgtccgtggtcaccgtgccgtcgtctagcctgggcacccagacctacatctgcaacgtgaaccacaagccgtccaacaccaaagtggataagcgggtggagccgaagtcctgcgataagacacacacgtgcccgccatgtccagcgcctgaattgcttggcggaccttccgtgttcctgttcccgcctaagcccaaggacaccttgatgattagccggactcccgaagtcacctgtgtggtggtggcagtgtcccacgaggaccccgaggtcaagtttaattggtacgtggacggcgtcgaagtgcacaacgccaagactaagccccgggaggaacagtacaacagcacctaccgggtcgtgtccgtgctgaccgtgctgcaccaggactggctgaatgggaaagagtacaagtgcaaagtgtccaacaaggccttggccgctcctatcgaaaaaactatcagcaaggctaagggacagccgagggaaccccaagtctacaccctgcccccttcacgcgaagagatgaccaagaatcaagtgtcgctgacctgcctcgtcaagggattctacccctccgacattgcggtggagtgggagtccaacggccagcccgagaacaactacaagactactccgcccgtgctggactccgacggcagcttcttcctgtattccaagctgaccgtggacaagtcccggtggcagcaaggaaacgtgttctcctgctcggtcatgcacgaagccctgcacaaccactatacgcagaagtccctgtccttgagcccggggaaa

SEQ ID NO: 17 - protein, 11 aa, Homo sapiens
RASQGISSYLA

SEQ ID NO: 18 - protein, 7 aa, Homo sapiens
TASTLQS

SEQ ID NO: 19 - protein, 9 aa, Homo sapiens
QQTWRKPRT

SEQ ID NO: 20 - protein, 7 aa, Homo sapiens
SQGISSY

SEQ ID NO: 21 - skipped

SEQ ID NO: 22 - protein, 6 aa, Homo sapiens
TWRKPR

SEQ ID NO: 23 - protein, 6 aa, Homo sapiens
QGISSY

SEQ ID NO: 24 - protein, 107 aa, Homo sapiens
DIQMTQSPSSLSASVGDRVTITCRASQGISSYLAWYQQKPGKAPKLLIYTASTLQSGVPSRFSGSGSGTDFTLTISSLQPEDFATYYCQQTWRKPRTFGQGTKVEIK

SEQ ID NO: 25 - DNA, 321 bp, Homo sapiens
  source 1..321: mol_type = other DNA
gacatccagatgacccagtctccgtcctccctgtccgcatcagtgggggacagagtgaccatcacttgtcgggcctcccaaggcatctcgtcatacctggcctggtatcagcagaaacccggaaaggctccaaagctgctcatctacaccgcctcgactctgcaatccggagtgccttcccgcttctccggatccggttcgggaaccgacttcaccctcaccattagcagccttcagccggaagatttcgcgacctactactgccagcaaacctggcggaagcccaggacatttggccagggcactaaggtcgagattaag

SEQ ID NO: 26 - protein, 214 aa, Homo sapiens
DIQMTQSPSSLSASVGDRVTITCRASQGISSYLAWYQQKPGKAPKLLIYTASTLQSGVPSRFSGSGSGTDFTLTISSLQPEDFATYYCQQTWRKPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - DNA, 642 bp, Homo sapiens
  source 1..642: mol_type = other DNA
gacatccagatgacccagtctccgtcctccctgtccgcatcagtgggggacagagtgaccatcacttgtcgggcctcccaaggcatctcgtcatacctggcctggtatcagcagaaacccggaaaggctccaaagctgctcatctacaccgcctcgactctgcaatccggagtgccttcccgcttctccggatccggttcgggaaccgacttcaccctcaccattagcagccttcagccggaagatttcgcgacctactactgccagcaaacctggcggaagcccaggacatttggccagggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 28 - protein, 125 aa, Homo sapiens
QVQLLESGGGLVQPGGSLRLSCAASGFTFSSYWMNWVRQAPGKGLEWVSVIESKGNYIFYADSVKGRFTISRDNSKNTLYLQMNSLRAEDTAVYYCARDRYSMIYSYGAGAFDYWGQGTLVTVSS

SEQ ID NO: 29 - DNA, 375 bp, Homo sapiens
  source 1..375: mol_type = other DNA
caagttcaattgctggaaagcggaggtggacttgtccaacctggagggtcactccgactgtcttgcgctgcatcaggattcacctttagtagctattggatgaactgggtccggcaggctcctgggaaagggcttgagtgggtaagtgtcattgaatcaaagggcaactacatcttttatgctgattctgtaaagggtaggttcaccatctccagggacaattcaaaaaatactttgtatctgcagatgaactctctcagggcagaagacacggccgtttattactgcgcccgcgatcgatacagcatgatatactcctacggcgcaggagcttttgactactggggtcaaggcacacttgttactgtcagtagc

SEQ ID NO: 30 - protein, 455 aa, Homo sapiens
QVQLLESGGGLVQPGGSLRLSCAASGFTFSSYWMNWVRQAPGKGLEWVSVIESKGNYIFYADSVKGRFTISRDNSKNTLYLQMNSLRAEDTAVYYCARDRYSMIYSYGAGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - DNA, 1365 bp, Homo sapiens
  source 1..1365: mol_type = other DNA
caagttcaattgctggaaagcggaggtggacttgtccaacctggagggtcactccgactgtcttgcgctgcatcaggattcacctttagtagctattggatgaactgggtccggcaggctcctgggaaagggcttgagtgggtaagtgtcattgaatcaaagggcaactacatcttttatgctgattctgtaaagggtaggttcaccatctccagggacaattcaaaaaatactttgtatctgcagatgaactctctcagggcagaagacacggccgtttattactgcgcccgcgatcgatacagcatgatatactcctacggcgcaggagcttttgactactggggtcaaggcacacttgttactgtcagtagcgcctcaacgaaaggaccgtccgtgtttcctcttgctcctagctccaaatccacctcaggtggaacggccgccctggggtgcctggtaaaggactatttcccagagccagttactgtgtcttggaattctggtgcattgacaagtggcgtacacacttttcccgcggtcctccaatctagtggtctgtactcactgtcctccgttgtgactgtcccaagtagctcacttggcacacagacttacatctgtaatgttaatcataagccgtcaaacacgaaggtggataagagggtagaacctaagtcatgtgacaaaacgcatacttgccccccctgccctgcgccggaagccgctggcggaccctccgtattcttgttccctccaaagccaaaggacactctgatgattagccggacaccggaggtcacttgtgttgtagttgacgtcagccatgaggatcctgaggtgaaatttaattggtacgtggacggggttgaagtccacaatgctaaaactaaacctagggaagagcaatataatagtacatacagggttgtcagtgtgctgaccgttctccatcaggactggctgaacggcaaggaatacaagtgcaaggtcagcaacaaggccttgccggcccccatcgagaagacgatctccaaagccaaggggcaaccccgagaaccgcaggtatacacgctcccccctagtagagaagagatgacaaagaatcaagtttccttgacgtgccttgtgaaaggcttctaccctagtgacatcgcagtcgaatgggagagcaacgggcagccggagaataactataaaacaaccccccccgtgcttgactcagacgggtcattttttctgtatagcaaattgactgttgataaatcacggtggcaacaaggaaacgtgtttagttgcagcgtaatgcacgaagctctccacaatcactatactcaaaagtcactgtcactctcccctggcaag

SEQ ID NO: 32 - DNA, 321 bp, Homo sapiens
  source 1..321: mol_type = other DNA
gacattcaaatgacacaaagtccgtccagtcttagtgcttctgtgggcgatagggtcaccatcacttgtcgggcgtctcaggggatcagctcttacttggcatggtatcaacaaaagccaggaaaagcacctaaattgcttatttatacagcgtccaccctccagtcaggagtgcctagtaggttctcaggctctgggtccggtactgacttcacgctgactatatcaagcttgcaacccgaagattttgcaacatactactgccaacagacatggaggaagccaagaactttcggtcagggaacgaaagttgagataaag

SEQ ID NO: 33 - DNA, 642 bp, Homo sapiens
  source 1..642: mol_type = other DNA
gacattcaaatgacacaaagtccgtccagtcttagtgcttctgtgggcgatagggtcaccatcacttgtcgggcgtctcaggggatcagctcttacttggcatggtatcaacaaaagccaggaaaagcacctaaattgcttatttatacagcgtccaccctccagtcaggagtgcctagtaggttctcaggctctgggtccggtactgacttcacgctgactatatcaagcttgcaacccgaagattttgcaacatactactgccaacagacatggaggaagccaagaactttcggtcagggaacgaaagttgagataaagcgcactgtcgcagcaccttccgtgttcattttcccgccttccgacgagcagcttaaatcagggaccgcgagtgttgtttgcttgcttaataacttttacccacgggaagccaaagttcagtggaaggtggacaatgcactccaaagcgggaatagtcaggagtcagttactgagcaagatagtaaagactctacttactctttgagttcaaccttgaccctctcaaaagcggactacgagaagcataaagtgtacgcctgcgaggtgacgcatcaaggtttgtcttccccggttacgaagtcctttaataggggggaatgt

SEQ ID NO: 34 - protein, 9 aa, Homo sapiens
QQEWVKPRT

SEQ ID NO: 35 - protein, 6 aa, Homo sapiens
EWVKPR

SEQ ID NO: 36 - protein, 107 aa, Homo sapiens
DIQMTQSPSSLSASVGDRVTITCRASQGISSYLAWYQQKPGKAPKLLIYTASTLQSGVPSRFSGSGSGTDFTLTISSLQPEDFATYYCQQEWVKPRTFGQGTKVEIK

SEQ ID NO: 37 - DNA, 321 bp, Homo sapiens
  source 1..321: mol_type = other DNA
gacatccagatgacccagtctccgtcctccctgtccgcatcagtgggggacagagtgaccatcacttgtcgggcctcccaaggcatctcgtcatacctggcctggtatcagcagaaacccggaaaggctccaaagctgctcatctacaccgcctcgactctgcaatccggagtgccttcccgcttctccggatccggttcgggaaccgacttcaccctcaccattagcagccttcagccggaagatttcgcgacctactactgccagcaagaatgggtgaagcccaggacatttggccagggcactaaggtcgagattaag

SEQ ID NO: 38 - protein, 214 aa, Homo sapiens
DIQMTQSPSSLSASVGDRVTITCRASQGISSYLAWYQQKPGKAPKLLIYTASTLQSGVPSRFSGSGSGTDFTLTISSLQPEDFATYYCQQEWVKPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - DNA, 642 bp, Homo sapiens
  source 1..642: mol_type = other DNA
gacatccagatgacccagtctccgtcctccctgtccgcatcagtgggggacagagtgaccatcacttgtcgggcctcccaaggcatctcgtcatacctggcctggtatcagcagaaacccggaaaggctccaaagctgctcatctacaccgcctcgactctgcaatccggagtgccttcccgcttctccggatccggttcgggaaccgacttcaccctcaccattagcagccttcagccggaagatttcgcgacctactactgccagcaagaatgggtgaagcccaggacatttggccagggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 40 - DNA, 321 bp, Homo sapiens
  source 1..321: mol_type = other DNA
gacatacaaatgacgcaatctccgagtagcttgtcagcgtccgtaggcgaccgagtaacgattacgtgtagagcgagccagggaatttcatcttatttggcttggtatcagcaaaagccgggaaaagcacccaaactcctcatttatactgccagcacgttgcaaagcggcgttccgagtcggttctctggatcagggtccgggacggacttcaccttgacgatttcatctttgcaacctgaagattttgcaacatactactgtcaacaggagtgggtgaagccaaggaccttcggacaaggcacgaaggtcgaaatcaag

SEQ ID NO: 41 - DNA, 642 bp, Homo sapiens
  source 1..642: mol_type = other DNA
gacatacaaatgacgcaatctccgagtagcttgtcagcgtccgtaggcgaccgagtaacgattacgtgtagagcgagccagggaatttcatcttatttggcttggtatcagcaaaagccgggaaaagcacccaaactcctcatttatactgccagcacgttgcaaagcggcgttccgagtcggttctctggatcagggtccgggacggacttcaccttgacgatttcatctttgcaacctgaagattttgcaacatactactgtcaacaggagtgggtgaagccaaggaccttcggacaaggcacgaaggtcgaaatcaagcgaaccgtggcagctccgtccgtgtttatttttccgccttccgacgaacaacttaaaagtggaacagcctctgtcgtctgtctccttaacaacttctaccccagggaagctaaagtacagtggaaggtagataacgctctgcaaagtggtaattctcaggagagcgtcacggaacaggactccaaagactccacctattctctgagctctacactgacgctcagcaaggcagactacgaaaagcacaaagtatatgcgtgtgaggtgacgcatcaaggccttagcagtccagttacaaaaagttttaacaggggagaatgc

(19) United States
(12) Patent Application Publication
(10) Pub. No.: US 2025/0223370 A1
(43) Pub. Date: Jul. 10, 2025

(54) METHODS OF TREATING DISORDERS USING ANTI-NATRIURETIC PEPTIDE RECEPTOR 1 (NPR1) ANTIBODIES

(71) Applicant: NOVARTIS AG, Basel (CH)

(72) Inventors: Yuichiro ADACHI, Arlington, MA (US); John Louis DIENER, Cambridge, MA (US); Robert J. A. FROST, Allschwil (CH); Yan-Ling HE, Belmont, MA (US); Venkateswar JARUGULA, Belle Mead, NJ (US); Adel R. RIZKALA, East Brunswick, NJ (US); Cesare RUSSO, Basel (CH); Xueping WU, Schriesheim (DE)

(21) Appl. No.: 18/851,514

(22) PCT Filed: Mar. 28, 2023
(86) PCT No.: PCT/IB2023/053094

§ 371 Date: Sep. 26, 2024

Related U.S. Application Data
(60) Provisional application No. 63/325,208, filed on Mar. 30, 2022.
(60) Provisional application No. 63/408,669, filed on Sep. 21, 2022.
(60) Provisional application No. 63/482,619, filed on Feb. 1, 2023.

(51) Int. Cl. C07K 16/28

(52) U.S. Cl. CPC C07K 16/2869

(57) ABSTRACT

Described herein are methods of using an anti-natriuretic peptide receptor 1 (NPR1) antibody or antigen binding fragment thereof for the treatment or prevention of a disease or disorder in a human, in particular for a disorder or a disease associated with natriuretic peptide receptor activity (including, but not limited to heart failure, e.g., HFrEF, HFmrEF, or HFpEF; or hypertension, e.g., resistant hypertension).

### FIELD

Described herein are methods of using an anti-NPR1 antibodies or antigen binding fragments thereof for the treatment of one or more conditions including cardiovascular diseases such as heart failure (e.g., heart failure with reduced ejection fraction, heart failure with mid-range ejection fraction or heart failure with mildly reduced ejection fraction, heart failure with preserved ejection fraction); and/or hypertension (e.g., arterial hypertension, resistant hypertension).

### BACKGROUND

The natriuretic peptide (NP) system is an endocrine, autocrine, and paracrine system involved in the homeostasis of intravascular volume and blood pressure. Natriuretic Peptide Receptor 1 (NPR1; also known as NPRA) is a membrane-bound particulate guanylate cyclase and is the receptor for natriuretic peptides ANP and BNP. See: Kuhn (2003) Circ. Res. p. 700-9; and Boerrigter and Burnett (2004) Expert Opin Investig Drugs p. 643-52. Binding of ANP and BNP to NPR1 stimulates an intracellular cGMP-dependent signaling cascade, which mediates the physiological actions of ANP and BNP. ANP and BNP play a pivotal role in the regulation of blood pressure (BP) and intravascular volume. NPs act on the regulation of vascular tone via a direct relaxant effect on vascular smooth muscle cells. Furthermore, the NPs have been shown to suppress the renin-angiotensin-aldosterone system (RAAS), reduce the sympathetic tone, and inhibit secretion of the vasoconstrictor endothelin-1 (ET-1). See: Nishikimi et al. (2006) Cardiovasc. Res. p. 318-28. NPs also regulate intravascular volume through a natriuretic effect in the kidneys and mediating direct effects on endothelial permeability in the vasculature. See: Curry (2005) J. Clin. Invest. p. 1458-61. Moreover, ANP has shown direct myocardial antihypertropic and antifibrotic properties. See: Calderone et al (1998) J. Clin. Invest. p. 812-8; Patel et al (2005) Am. J. Physiol. Heart Circ. Physiol. p. H777-84; Nishikimi et al. (2006) Cardiovasc. Res. p. 318-28; Ichiki et al. (2014) J. Mol. Cell. Cardiol. p. 199-205; and Theilig and Wu (2015) Am. J. Physiol. Renal Physiol. p. F1047-55.

Heart failure (HF) is a major public health problem concerning more than 20 million patients around the world (Orso et al., 2014, Expert Opin Pharmacother. 15 (13): 1849-1861) and is associated with high morbidity (Ibebuogu et al., 2011, Circulation. Heart failure 4 (2): 114-120). In the United States alone, over 6 million patients have heart failure, and around half of them have a reduced left venticular ejection fraction (HFrEF). Recently, sacubatril-valsartan has been shown to lead to significant improvement in cardiovascular outcomes with HFrEF and in those with lower than normal ejection fraction (EF). See: McMurray et al. (2014) N Engl J Med p. 993-1004 and Solomon et al. (2019) N Engl J Med p. 1609-1620. Treatment with sacubatril-valsartan results in an indirect stimulation of NPR1 by increasing the circulating levels of ANP and BNP through the inhibition of neprilysin, the enzyme responsible for degrading the NPs.

Certain antibodies against NPR1 have been described, for example, in WO2010/065293, WO2020/086406, and WO2020/250159, the contents of each of which are herein incorporated by reference for this purpose. However, there is a need for specific methods of treatment and uses to supplement or replace existing therapies for disorders or diseases associated with natriuretic peptide receptor 1 (NPR1) activity such as cardiovascular diseases. Such cardiovascular diseases include, but are not limited to: heart failure (e.g., heart failure with reduced ejection fraction, heart failure with mid-range ejection fraction or heart failure with mildly reduced ejection fraction, or heart failure with preserved ejection fraction); and/or hypertension (e.g., arterial hypertension, resistant hypertension). Additionally, there is a need for specific methods of treatment and uses to supplement or replace existing therapies for other disorders or diseases associated with natriuretic peptide receptor 1 (NPR1) activity including, but not limited to, preeclampsia, asthma, glaucoma, cytokine release syndrome and/or kidney disorders.

### SUMMARY

The methods and uses presented herein describe administration of anti-NPR1 antibodies or antigen binding fragments thereof that are agonists of NPR1 to supplement or replace existing therapies for disorders or diseases associated with natriuretic peptide receptor 1 (NPR1) activity (e.g., cardiovascular diseases including, but not limited to: heart failure (e.g., heart failure with reduced ejection fraction, heart failure with mid-range ejection fraction or heart failure with mildly reduced ejection fraction, or heart failure with preserved ejection fraction); and/or hypertension (e.g., arterial hypertension or resistant hypertension). The action of the described antibodies or antigen binding fragments thereof is specific for NPR1, because such antibodies or antigen binding fragments thereof selectively bind to and activate NPR1, but do not either bind to NPR3 and/or activate NPR2 in vitro. The described antibodies replicate the action of ANP (increasing plasma cGMP, lowering BP and increasing diuresis) in non-clinical pharmacology studies. The described antibodies can effectively replace ANP in ANP knockout mice, reversing both cardiac hypertrophy and elevations in NT-proBNP. The described antibodies also increase plasma cGMP and lower blood pressure in cynomolgus monkeys. These preclinical data, together with evidence in humans of the potential beneficial effects of ANP and other ANP mimetics on blood pressure and pulmonary wedge pressure, provide a scientific rationale for methods and uses of the antibodies described herein for certain conditions which may be treatable with NPR1 agonists, including, but not limited to, cardiovascular diseases such as heart failure (e.g., heart failure with reduced ejection fraction, heart failure with mid-range ejection fraction or heart failure with mildly reduced ejection fraction, or heart failure with preserved ejection fraction); and/or hypertension (e.g., arterial hypertension, resistant hypertension).

In one aspect, presented herein is a method of treating a disorder or a disease associated with natriuretic peptide receptor 1 (NPR1) activity in a subject in need thereof, the method comprising administering to the subject about 50 mg to about 700 mg of an anti-NPR1 antibody or antigen binding fragment thereof.

In certain embodiments, the disorder or disease associated with natriuretic peptide receptor activity is a cardiovascular disorder. In certain embodiments, the cardiovascular disorder is selected from: hypertension, peripheral vascular disease, heart failure, coronary artery disease (CAD), ischemic heart disease (IHD), mitral stenosis and regurgitation, angina, hypertrophic cardiomyopathy, diabetic cardiomyopathy, supraventricular and ventricular arrhythmias, cardiac dysrhythmia, atrial fibrillation (AF), new onset of atrial fibrillation, recurrent atrial fibrillation, cardiac fibrosis, atrial flutter, detrimental vascular remodeling, plaque stabilization, and myocardial infarction (MI). In some embodiments, the subject has hypertrophic cardiomyopathy, wherein the hypertrophic cardiomyopathy is ventricular hypertrophy. In a specific embodiment, the cardiovascular disorder is heart failure. In some embodiments, the heart failure is selected from heart failure with reduced ejection fraction (HFrEF), heart failure with mid-range ejection fraction or heart failure with mildly reduced ejection fraction (HFmrEF), heart failure with preserved ejection fraction (HFpEF), heart failure after acute myocardial infarct, or acute decompensated heart failure. In a specific embodiment, the heart failure is heart failure with reduced ejection fraction (HFrEF). In a specific embodiment, the heart failure is heart failure with mid-range ejection fraction or heart failure with mildly reduced ejection fraction or heart failure with mildly reduced ejection fraction (HFmrEF). In some embodiments, the cardiovascular disorder is hypertension. In certain embodiments, the hypertension is selected from resistant hypertension, hypertensive heart disease, pulmonary hypertension, isolated systolic hypertension, and pulmonary arterial hypertension. In a specific embodiment, the hypertension is resistant hypertension.

In some embodiments, the disorder or disease associated with natriuretic peptide receptor activity is selected from hypertension, preeclampsia, asthma, glaucoma, and/or cytokine release syndrome. In certain embodiments, the disorder or disease associated with natriuretic peptide receptor activity is a kidney disorder. In specific embodiments, the kidney disorder is selected from: diabetic renal insufficiency, non-diabetic renal insufficiency, renal failure, diabetic nephropathy, non-diabetic nephropathy, acute renal injury, contrast induced nephropathy, nephrotic syndrome, glomerulonephritis, scleroderma, glomerular sclerosis, proteinuria of primary renal disease, renal vascular hypertension, diabetic retinopathy and end-stage renal disease (ESRD), endothelial dysfunction, diastolic dysfunction, renal fibrosis, and polycystic kidney disease (PKD).

In some embodiments, the anti-NPR1 antibody or antigen binding fragment thereof comprises three heavy chain complementarity determining regions (HCDR1, HCDR2, and HCDR3) and three light chain complementarity determining regions (LCDR1, LCDR2, and LCDR3) selected from: (I) SEQ ID NO: 4 (HCDR1), SEQ ID NO: 5 (HCDR2), SEQ ID NO: 6 (HCDR3), SEQ ID NO: 17 (LCDR1), SEQ ID NO: 18 (LCDR2), and SEQ ID NO: 19 (LCDR3); (II) SEQ ID NO: 7 (HCDR1), SEQ ID NO: 5 (HCDR2), SEQ ID NO: 6 (HCDR3), SEQ ID NO: 17 (LCDR1), SEQ ID NO: 18 (LCDR2), and SEQ ID NO: 19 (LCDR3); (III) SEQ ID NO: 8 (HCDR1), SEQ ID NO: 9 (HCDR2), SEQ ID NO: 6 (HCDR3), SEQ ID NO: 20 (LCDR1), SEQ ID NO: 21 (LCDR2), and SEQ ID NO: 22 (LCDR3); or (IV) SEQ ID NO: 10 (HCDR1), SEQ ID NO: 11 (HCDR2), SEQ ID NO: 12 (HCDR3), SEQ ID NO: 23 (LCDR1), SEQ ID NO: 21 (LCDR2), and SEQ ID NO: 19 (LCDR3). In certain embodiments, the anti-NPR1 antibody or antigen binding fragment thereof comprises: (a) a heavy chain variable region comprising an amino acid sequence having at least 95%, 96%, 97%, 98%, or 99% identity with SEQ ID NO: 13, and a light chain variable region comprising an amino acid sequence having at least 95%, 96%, 97%, 98%, or 99% identity with SEQ ID NO: 24; or (b) a heavy chain variable region comprising an amino acid sequence having at least 95%, 96%, 97%, 98%, or 99% identity with SEQ ID NO: 28, and a light chain variable region comprising an amino acid sequence having at least 95%, 96%, 97%, 98%, or 99% identity with SEQ ID NO: 24. In certain embodiments, the anti-NPR1 antibody or antigen binding fragment thereof comprises: (a) a heavy chain variable region comprising an amino acid sequence of SEQ ID NO: 13, and a light chain variable region comprising an amino acid sequence of SEQ ID NO: 24; or (b) a heavy chain variable region comprising an amino acid sequence of SEQ ID NO: 28, and a light chain variable region comprising an amino acid sequence of SEQ ID NO: 24. In certain embodiments, the anti-NPR1 antibody or antigen binding fragment thereof comprises: (a) a heavy chain comprising an amino acid sequence having at least 95%, 96%, 97%, 98%, or 99% identity with SEQ ID NO: 15, and a light chain comprising an amino acid sequence having at least 95%, 96%, 97%, 98%, or 99% identity with SEQ ID NO: 26; or (b) a heavy chain comprising an amino acid sequence having at least 95%, 96%, 97%, 98%, or 99% identity with SEQ ID NO: 30, and a light chain comprising an amino acid sequence having at least 95%, 96%, 97%, 98%, or 99% identity with SEQ ID NO: 26. In certain embodiments, the anti-NPR1 antibody or antigen binding fragment thereof comprises: (a) a heavy chain comprising an amino acid sequence of SEQ ID NO: 15, and a light chain comprising an amino acid sequence of SEQ ID NO: 26; or (b) a heavy chain comprising an amino acid sequence of SEQ ID NO: 30, and a light chain comprising an amino acid sequence of SEQ ID NO: 26.

In certain embodiments, the anti-NPR1 antibody or antigen binding fragment thereof comprises three heavy chain complementarity determining regions (HCDR1, HCDR2, and HCDR3) and three light chain complementarity determining regions (LCDR1, LCDR2, and LCDR3) selected from: (I) SEQ ID NO: 4 (HCDR1), SEQ ID NO: 5 (HCDR2), SEQ ID NO: 6 (HCDR3), SEQ ID NO: 17 (LCDR1), SEQ ID NO: 18 (LCDR2), and SEQ ID NO: 34 (LCDR3); (II) SEQ ID NO: 7 (HCDR1), SEQ ID NO: 5 (HCDR2), SEQ ID NO: 6 (HCDR3), SEQ ID NO: 17 (LCDR1), SEQ ID NO: 18 (LCDR2), and SEQ ID NO: 34 (LCDR3); (III) SEQ ID NO: 8 (HCDR1), SEQ ID NO: 9 (HCDR2), SEQ ID NO: 6 (HCDR3), SEQ ID NO: 20 (LCDR1), SEQ ID NO: 21 (LCDR2), and SEQ ID NO: 35 (LCDR3); or (IV) SEQ ID NO: 10 (HCDR1), SEQ ID NO: 11 (HCDR2), SEQ ID NO: 12 (HCDR3), SEQ ID NO: 23 (LCDR1), SEQ ID NO: 21 (LCDR2), and SEQ ID NO: 34 (LCDR3). In certain embodiments, the anti-NPR1 antibody or antigen binding fragment thereof comprises: (a) a heavy chain variable region comprising an amino acid sequence having at least 95%, 96%, 97%, 98%, or 99% identity with SEQ ID NO: 13, and a light chain variable region comprising an amino acid sequence having at least 95%, 96%, 97%, 98%, or 99% identity with SEQ ID NO: 36; or (b) a heavy chain variable region comprising an amino acid sequence having at least 95%, 96%, 97%, 98%, or 99% identity with SEQ ID NO: 28, and a light chain variable region comprising an amino acid sequence having at least 95%, 96%, 97%, 98%, or 99% identity with SEQ ID NO: 36. In certain embodiments, the anti-NPR1 antibody or antigen binding fragment thereof comprises: (a) a heavy chain variable region comprising an amino acid sequence of SEQ ID NO: 13, and a light chain variable region comprising an amino acid sequence of SEQ ID NO: 36; or (b) a heavy chain variable region comprising an amino acid sequence of SEQ ID NO: 28, and a light chain variable region comprising an amino acid sequence of SEQ ID NO: 36. In certain embodiments, the anti-NPR1 antibody or antigen binding fragment thereof comprises: (a) a heavy chain 15 comprising an amino acid sequence having at least 95%, 96%, 97%, 98%, or 99% identity with SEQ ID NO: 15, and a light chain comprising an amino acid sequence having at least 95%, 96%, 97%, 98%, or 99% identity with SEQ ID NO: 38; or (b) a heavy chain comprising an amino acid sequence having at least 95%, 96%, 97%, 98%, or 99% identity with SEQ ID NO: 30, and a light chain comprising an amino acid sequence having at least 95%, 96%, 97%, 98%, or 99% identity with SEQ ID NO: 38. In certain embodiments, the anti-NPR1 antibody or antigen binding fragment thereof comprises: (a) a heavy chain comprising an amino acid sequence of SEQ ID NO: 15, and a light chain comprising an amino acid sequence of SEQ ID NO: 38; or (b) a heavy chain comprising an amino acid sequence of SEQ ID NO: 30, and a light chain comprising an amino acid sequence of SEQ ID NO: 38.

In specific embodiments, the anti-NPR1 antibody or antigen binding fragment thereof is therapeutic. In some embodiments, the antigen binding fragment is selected from the group consisting of a Fab, Fab′, F(ab′)2, Fv, and a single chain variable fragment (scFv). In some embodiments, about 60 mg, about 120 mg, about 240 mg, about 450 mg, or about 600 mg of the anti-NPR1 antibody or antigen binding fragment thereof is administered to the subject. In some embodiments, about 120 mg or about 240 mg of the anti-NPR1 antibody or antigen binding fragment thereof is administered to the subject. In some embodiments, about 60 mg of the anti-NPR1 antibody or antigen binding fragment thereof is administered to the subject. In some embodiments, about 120 mg of the anti-NPR1 antibody or antigen binding fragment thereof is administered to the subject. In some embodiments about 240 mg of the anti-NPR1 antibody or antigen binding fragment thereof is administered to the subject. In some embodiments about 450 mg of the anti-NPR1 antibody or antigen binding fragment thereof is administered to the subject. In some embodiments about 600 mg of the anti-NPR1 antibody or antigen binding fragment thereof is administered to the subject. In certain embodiments, one or more additional therapeutically active agents are being administered to the subject. In certain embodiments, the one or more additional therapeutically active agents is selected from an ACE (angiotensin-converting-enzyme) inhibitor, an angiotensin receptor blocker (ARB), a neprilysin inhibitor, a mineralocorticoid receptor antagonist (MRA), a beta blocker, a diuretic, a calcium channel blocker, a cardiac glycoside, a sodium-glucose co-transporter 2 inhibitor (SGLT2i), an angiotensin receptor neprilysin inhibitor (ARNI), a corticosteroid, a leukotriene modifier, a bronchodilator, a beta-adrenoceptor antagonist, a carbonic anhydrase inhibitor, an alpha 2-adrenoceptor agonist, a parasympathomimetic, a prostaglandin analog, a rho kinase inhibitor, a cell therapy, a cardiac-specific myosin activator, and combinations thereof. In some embodiments, the one or more additional therapeutically active agents is an ACE (angiotensin-converting-enzyme) inhibitor, an angiotensin receptor blocker (ARB), a beta blocker, a diuretic, or combinations thereof. In some embodiments, the one or more additional therapeutically active agents is an ACE (angiotensin-converting-enzyme) inhibitor, an angiotensin receptor blocker (ARB), an angiotensin receptor neprilysin inhibitor (ARNI), a beta blocker, a mineralocorticoid receptor antagonist (MRA), a sodium-glucose co-transporter 2 inhibitor (SGLT2i), a diuretic, or combinations thereof. In some embodiments, the one or more additional therapeutically active agents is selected from enalapril, benazepril, captopril, fosinopril, lisinopril, moexipril, perindopril, quinapril, ramipril, trandolapril, valsartan, azilsartan, candesartan, eprosartan, irbesartan, losartan, olmesartan, telmisartan, sacubitril, bisoprolol, carvedilol, propanolol, metoprolol, metoprolol tartrate, metoprolol succinate, thiazide diuretics, loop diuretics, potassium-sparing diuretics, amlodipine, clevidipine, diltiazem, felodipine, isradipine, nicardipine, nifedipine, nisoldipine, verapamil, a digitalis glycoside, canagliflozin, dapagliflozin, empagliflozin, sotagliflozin, ertugliflozin, chlorothiazide, chlorthalidone, hydrochlorothiazide, indapamide, metolazone, bumetanide, ethacrynic acid, furosemide, torsemide, amiloride, eplerenone, finerone, spironolactone, triamterene, digoxin, fluticasone, budesonide, mometasone, beclomethasone, ciclesonide, fluticasone furoate, prednisone, methylprednisolone, montelukast, zafirlukast, zileuton, a long-acting beta agonist, a short-acting beta agonist, theophylline, ipratropium, salmeterol, formoterol, albuterol, levalbuterol, timolol, levobunolol, metipranolol, carteolol, betaxolol, acetazolamide, dorzolamide, brinzolamide, methazolamide, brimonidine, apraclonidine, a cholinomimetic, latanoprost, latanoprostene bunod, travoprost, bimatoprost, tafluprost, omecamtiv mecarbil, allogeneic cell therapy rexlemestrocel-L (REVASCOR®), CardiAMP™ Cell Therapy, netarsudil, ripasudil, and combinations thereof. In certain embodiments, the anti-NPR1 antibody or antigen binding fragment thereof is subcutaneously administered to the subject. In some embodiments, the anti-NPR1 antibody or antigen binding fragment thereof is administered about once every week, once every 2 weeks, about once every 3 weeks, about once every 4 weeks, about once every 5 weeks, about once every 6 weeks, about once every 7 weeks, about once every 8 weeks, about once every 9 weeks, about once every 10 weeks, about once every 11 weeks, or about once every 12 weeks. In some embodiments, the anti-NPR1 antibody or antigen binding fragment thereof is administered about once every week. In some embodiments, the anti-NPR1 antibody or antigen binding fragment thereof is administered about once every 2 weeks. In some embodiments, the anti-NPR1 antibody or antigen binding fragment thereof is administered about once every 3 weeks. In some embodiments, the anti-NPR1 antibody or antigen binding fragment thereof is administered about once every 4 weeks. In some embodiments, the anti-NPR1 antibody or antigen binding fragment thereof is administered about once every month. In some embodiments, the anti-NPR1 antibody or antigen binding fragment thereof is administered about once every 5 weeks. In some embodiments, the anti-NPR1 antibody or antigen binding fragment thereof is administered about once every 6 weeks. In some embodiments, the anti-NPR1 antibody or antigen binding fragment thereof is administered about once every 7 weeks. In some embodiments, the anti-NPR1 antibody or antigen binding fragment thereof is administered about once every 8 weeks. In some embodiments, the anti-NPR1 antibody or antigen binding fragment thereof is administered about once every two months. In some embodiments, the anti-NPR1 antibody or antigen binding fragment thereof is provided for administration about once every 9 weeks. In some embodiments, the anti-NPR1 antibody or antigen binding fragment thereof is provided for administration about once every 10 weeks. In some embodiments, the anti-NPR1 antibody or antigen binding fragment thereof is provided for administration about once every 11 weeks. In some embodiments, the anti-NPR1 antibody or antigen binding fragment thereof is provided for administration about once every 12 weeks. In some embodiments, the anti-NPR1 antibody or antigen binding fragment thereof is provided for administration about once every three months. In one aspect, provided herein is an anti-NPR1 antibody or antigen binding fragment thereof for use in a method of treating a disorder or a disease associated with natriuretic peptide receptor activity in a subject in need thereof, wherein the anti-NPR1 antibody or antigen binding fragment thereof is provided for administration at a dose in the range of about 10 mg to about 700 mg. In one aspect, provided herein is an anti-NPR1 antibody or antigen binding fragment thereof for use in a method of treating a disorder or a disease associated with natriuretic peptide receptor activity in a subject in need thereof, wherein the anti-NPR1 antibody or antigen binding fragment thereof is administered at a dose in the range of about 10 mg to about 700 mg. In one aspect, provided herein is an anti-NPR1 antibody or antigen binding fragment thereof for use in a method of treating a disorder or a disease associated with natriuretic peptide receptor activity in a subject in need thereof, wherein the anti-NPR1 antibody or antigen binding fragment thereof is provided for administration at a dose in the range of about 50 mg to about 700 mg. In one aspect, provided herein is an anti-NPR1 antibody or antigen binding fragment thereof for use in a method of treating a disorder or a disease associated with natriuretic peptide receptor activity in a subject in need thereof, wherein the anti-NPR1 antibody or antigen binding fragment thereof is administered at a dose in the range of about 50 mg to about 700 mg. In some embodiments, the disorder or disease associated with natriuretic peptide receptor activity is a cardiovascular disorder. In certain embodiments, the cardiovascular disorder is selected from: hypertension, peripheral vascular disease, heart failure, coronary artery disease (CAD), ischemic heart disease (IHD), mitral stenosis and regurgitation, angina, hypertrophic cardiomyopathy, diabetic cardiomyopathy, supraventricular and ventricular arrhythmias, cardiac dysrhythmia, atrial fibrillation (AF), new onset of atrial fibrillation, recurrent atrial fibrillation, cardiac fibrosis, atrial flutter, detrimental vascular remodeling, plaque stabilization, and myocardial infarction (MI). In some embodiments, the subject has hypertrophic cardiomyopathy, wherein the hypertrophic cardiomyopathy is ventricular hypertrophy. In some embodiments, the cardiovascular disorder is heart failure. In certain embodiments, the heart failure is selected from a heart failure with reduced ejection fraction (HFrEF), heart failure with mid-range ejection fraction or heart failure with mildly reduced ejection fraction (HFmrEF), heart failure with preserved ejection fraction (HFpEF), heart failure after acute myocardial infarct, or acute decompensated heart failure. In specific embodiments, the heart failure is heart failure with reduced ejection fraction (HFrEF). In specific embodiments, the heart failure is heart failure with mid-range ejection fraction or heart failure with mildly reduced ejection fraction or heart failure with mildly reduced ejection fraction (HFmrEF). In some embodiments, the cardiovascular disorder is hypertension. In certain embodiments, the hypertension is selected from resistant hypertension, hypertensive heart disease, pulmonary hypertension, isolated systolic hypertension, and pulmonary arterial hypertension. In a specific embodiment, the hypertension is resistant hypertension. In some embodiments, the disorder or disease associated with natriuretic peptide receptor activity is selected from hypertension, preeclampsia, asthma, glaucoma, and/or cytokine release syndrome. In certain embodiments, the disorder or disease associated with natriuretic peptide receptor activity is a kidney disorder. In certain embodiments, the kidney disorder is selected from: diabetic renal insufficiency, non-diabetic renal insufficiency, renal failure, diabetic nephropathy, non-diabetic nephropathy, acute renal injury, contrast induced nephropathy, nephrotic syndrome, glomerulonephritis, scleroderma, glomerular sclerosis, proteinuria of primary renal disease, renal vascular hypertension, diabetic retinopathy and end-stage renal disease (ESRD), endothelial dysfunction, diastolic dysfunction, renal fibrosis, and polycystic kidney disease (PKD). In some embodiments, the anti-NPR1 antibody or antigen binding fragment thereof comprises three heavy chain complementarity determining regions (HCDR1, HCDR2, and HCDR3) and three light chain complementarity determining regions (LCDR1, LCDR2, and LCDR3) selected from: (I) SEQ ID NO: 4 (HCDR1), SEQ ID NO: 5 (HCDR2), SEQ ID NO: 6 (HCDR3), SEQ ID NO: 17 (LCDR1), SEQ ID NO: 18 (LCDR2), and SEQ ID NO: 19 (LCDR3); (II) SEQ ID NO: 7 (HCDR1), SEQ ID NO: 5 (HCDR2), SEQ ID NO: 6 (HCDR3), SEQ ID NO: 17 (LCDR1), SEQ ID NO: 18 (LCDR2), and SEQ ID NO: 19 (LCDR3); (III) SEQ ID NO: 8 (HCDR1), SEQ ID NO: 9 (HCDR2), SEQ ID NO: 6 (HCDR3), SEQ ID NO: 20 (LCDR1), SEQ ID NO: 21 (LCDR2), and SEQ ID NO: 22 (LCDR3); or (IV) SEQ ID NO: 10 (HCDR1), SEQ ID NO: 11 (HCDR2), SEQ ID NO: 12 (HCDR3), SEQ ID NO: 23 (LCDR1), SEQ ID NO: 21 (LCDR2), and SEQ ID NO: 19 (LCDR3). In certain embodiments, the anti-NPR1 antibody or antigen binding fragment thereof comprises: (a) a heavy chain variable region comprising an amino acid sequence having at least 95%, 96%, 97%, 98%, or 99% identity with SEQ ID NO: 13, and a light chain variable region comprising an amino acid sequence having at least 95%, 96%, 97%, 98%, or 99% identity with SEQ ID NO: 24; or (b) a heavy chain variable region comprising an amino acid sequence having at least 95%, 96%, 97%, 98%, or 99% identity with SEQ ID NO: 28, and a light chain variable region comprising an amino acid sequence having at least 95%, 96%, 97%, 98%, or 99% identity with SEQ ID NO: 24. In certain embodiments, the anti-NPR1 antibody or antigen binding fragment thereof comprises: (a) a heavy chain variable region comprising an amino acid sequence of SEQ ID NO: 13, and a light chain variable region comprising an amino acid sequence of SEQ ID NO: 24; or (b) a heavy chain variable region comprising an amino acid sequence of SEQ ID NO: 28, and a light chain variable region comprising an amino acid sequence of SEQ ID NO: 24. In certain embodiments, the anti-NPR1 antibody or antigen binding fragment thereof comprises: (a) a heavy chain comprising an amino acid sequence having at least 95%, 96%, 97%, 98%, or 99% identity with SEQ ID NO: 15, and a light chain comprising an amino acid sequence having at least 95%, 96%, 97%, 98%, or 99% identity with SEQ ID NO: 26; or (b) a heavy chain comprising an amino acid sequence having at least 95%, 96%, 97%, 98%, or 99% identity with SEQ ID NO: 30, and a light chain comprising an amino acid sequence having at least 95%, 96%, 97%, 98%, or 99% identity with SEQ ID NO: 26. In certain embodiments, the anti-NPR1 antibody or antigen binding fragment thereof comprises: (a) a heavy chain comprising an amino acid sequence of SEQ ID NO: 15, and a light chain comprising an amino acid sequence of SEQ ID NO: 26; or (b) a heavy chain comprising an amino acid sequence of SEQ ID NO: 30, and a light chain comprising an amino acid sequence of SEQ ID NO: 26. In certain embodiments, the anti-NPR1 antibody or antigen binding fragment thereof comprises three heavy chain complementarity determining regions (HCDR1, HCDR2, and HCDR3) and three light chain complementarity determining regions (LCDR1, LCDR2, and LCDR3) selected from: (I) SEQ ID NO: 4 (HCDR1), SEQ ID NO: 5 (HCDR2), SEQ ID NO: 6 (HCDR3), SEQ ID NO: 17 (LCDR1), SEQ ID NO: 18 (LCDR2), and SEQ ID NO: 34 (LCDR3); (II) SEQ ID NO: 7 (HCDR1), SEQ ID NO: 5 (HCDR2), SEQ ID NO: 6 (HCDR3), SEQ ID NO: 17 (LCDR1), SEQ ID NO: 18 (LCDR2), and SEQ ID NO: 34 (LCDR3); (III) SEQ ID NO: 8 (HCDR1), SEQ ID NO: 9 (HCDR2), SEQ ID NO: 6 (HCDR3), SEQ ID NO: 20 (LCDR1), SEQ ID NO: 21 (LCDR2), and SEQ ID NO: 35 (LCDR3); or (IV) SEQ ID NO: 10 (HCDR1), SEQ ID NO: 11 (HCDR2), SEQ ID NO: 12 (HCDR3), SEQ ID NO: 23 (LCDR1), SEQ ID NO: 21 (LCDR2), and SEQ ID NO: 34 (LCDR3). In certain embodiments, the anti-NPR1 antibody or antigen binding fragment thereof comprises: (a) a heavy chain variable region comprising an amino acid sequence having at least 95%, 96%, 97%, 98%, or 99% identity with SEQ ID NO: 13, and a light chain variable region comprising an amino acid sequence having at least 95%, 96%, 97%, 98%, or 99% identity with SEQ ID NO: 36; or (b) a heavy chain variable region comprising an amino acid sequence having at least 95%, 96%, 97%, 98%, or 99% identity with SEQ ID NO: 28, and a light chain variable region comprising an amino acid sequence having at least 95%, 96%, 97%, 98%, or 99% identity with SEQ ID NO: 36. In certain embodiments, the anti-NPR1 antibody or antigen binding fragment thereof comprises: (a) a heavy chain variable region comprising an amino acid sequence of SEQ ID NO: 13, and a light chain variable region comprising an amino acid sequence of SEQ ID NO: 36; or (b) a heavy chain variable region comprising an amino acid sequence of SEQ ID NO: 28, and a light chain variable region comprising an amino acid sequence of SEQ ID NO: 36. In certain embodiments, the anti-NPR1 antibody or antigen binding fragment thereof comprises: (a) a heavy chain comprising an amino acid sequence having at least 95%, 96%, 97%, 98%, or 99% identity with SEQ ID NO: 15, and a light chain comprising an amino acid sequence having at least 95%, 96%, 97%, 98%, or 99% identity with SEQ ID NO: 38; or (b) a heavy chain comprising an amino acid sequence having at least 95%, 96%, 97%, 98%, or 99% identity with SEQ ID NO: 30, and a light chain comprising an amino acid sequence having at least 95%, 96%, 97%, 98%, or 99% identity with SEQ ID NO: 38. In certain embodiments, the anti-NPR1 antibody or antigen binding fragment thereof comprises: (a) a heavy chain comprising an amino acid sequence of SEQ ID NO: 15, and a light chain comprising an amino acid sequence of SEQ ID NO: 38; or (b) a heavy chain comprising an amino acid sequence of SEQ ID NO: 30, and a light chain comprising an amino acid sequence of SEQ ID NO: 38. In specific embodiments, the anti-NPR1 antibody or antigen binding fragment thereof is therapeutic. In some embodiments, the antigen binding fragment is selected from the group consisting of a Fab, Fab′, F(ab′)2, Fv, and a single chain variable fragment (scFv). In some embodiments, about 60 mg, about 120 mg, about 240 mg, about 450 mg, or about 600 mg of the anti-NPR1 antibody or antigen binding fragment thereof is administered to the subject. In some embodiments, about 120 mg or about 240 mg of the anti-NPR1 antibody or antigen binding fragment thereof is administered to the subject. In some embodiments, the anti-NPR1 antibody or antigen binding fragment thereof is provided at a dose of about 60 mg, about 120 mg, about 240 mg, about 450 mg, or about 600 mg. In some embodiments, about 60 mg of the anti-NPR1 antibody or antigen binding fragment thereof is administered to the subject. In some embodiments, about 120 mg of the anti-NPR1 antibody or antigen binding fragment thereof is administered to the subject. In some embodiments about 240 mg of the anti-NPR1 antibody or antigen binding fragment thereof is administered to the subject. In some embodiments about 450 mg of the anti-NPR1 antibody or antigen binding fragment thereof is administered to the subject. In some embodiments about 600 mg of the anti-NPR1 antibody or antigen binding fragment thereof is administered to the subject. In some embodiments, the anti-NPR1 antibody or antigen binding fragment thereof is provided at a dose of about 120 mg or about 240 mg. In some embodiments, the anti-NPR1 antibody or antigen binding fragment thereof is provided at a dose of about 120 mg. In some embodiments, the anti-NPR1 antibody or antigen binding fragment thereof is provided at a dose of about 240 mg. In some embodiments, the anti-NPR1 antibody or antigen binding fragment thereof is provided in combination with one or more additional therapeutically active agents. In certain embodiments, one or more additional therapeutically active agents are being administered to the subject. In certain embodiments, the one or more additional therapeutically active agents is selected from an ACE (angiotensin-converting-enzyme) inhibitor, an angiotensin receptor blocker (ARB), a neprilysin inhibitor, a mineralocorticoid receptor antagonist (MRA), a beta blocker, a diuretic, a calcium channel blocker, a cardiac glycoside, a sodium-glucose co-transporter 2 inhibitor (SGLT2i), an angiotensin receptor neprilysin inhibitor (ARNI), a corticosteroid, a leukotriene modifier, a bronchodilator, a beta-adrenoceptor antagonist, a carbonic anhydrase inhibitor, an alpha 2-adrenoceptor agonist, a parasympathomimetic, a prostaglandin analog, a rho kinase inhibitor, a cell therapy, a cardiac-specific myosin activator, and combinations thereof. In some embodiments, the one or more additional therapeutically active agents is an ACE (angiotensin-converting-enzyme) inhibitor, an angiotensin receptor blocker (ARB), a beta blocker, a diuretic, or combinations thereof. In some embodiments, the one or more additional therapeutically active agents is an ACE (angiotensin-converting-enzyme) inhibitor, an angiotensin receptor blocker (ARB), an angiotensin receptor neprilysin inhibitor (ARNI), a beta blocker, a mineralocorticoid receptor antagonist (MRA), a sodium-glucose co-transporter 2 inhibitor (SGLT2i), a diuretic, or combinations thereof. In some embodiments, the one or more additional therapeutically active agents is selected from enalapril, benazepril, captopril, fosinopril, lisinopril, moexipril, perindopril, quinapril, ramipril, trandolapril, valsartan, azilsartan, candesartan, eprosartan, irbesartan, losartan, olmesartan, telmisartan, sacubitril, bisoprolol, carvedilol, propanolol, metoprolol, metoprolol tartrate, metoprolol succinate, thiazide diuretics, loop diuretics, potassium-sparing diuretics, amlodipine, clevidipine, diltiazem, felodipine, isradipine, nicardipine, nifedipine, nisoldipine, verapamil, a digitalis glycoside, canagliflozin, dapagliflozin, empagliflozin, sotagliflozin, ertugliflozin, chlorothiazide, chlorthalidone, hydrochlorothiazide, indapamide, metolazone, bumetanide, ethacrynic acid, furosemide, torsemide, amiloride, eplerenone, finerone, spironolactone, triamterene, digoxin, fluticasone, budesonide, mometasone, beclomethasone, ciclesonide, fluticasone furoate, prednisone, methylprednisolone, montelukast, zafirlukast, zileuton, a long-acting beta agonist, a short-acting beta agonist, theophylline, ipratropium, salmeterol, formoterol, albuterol, levalbuterol, timolol, levobunolol, metipranolol, carteolol, betaxolol, acetazolamide, dorzolamide, brinzolamide, methazolamide, brimonidine, apraclonidine, a cholinomimetic, latanoprost, latanoprostene bunod, travoprost, bimatoprost, tafluprost, omecamtiv mecarbil, allogeneic cell therapy rexlemestrocel-L (REVASCOR®), CardiAMP™ Cell Therapy, netarsudil, ripasudil, and combinations thereof. In certain embodiments, the anti-NPR1 antibody or antigen binding fragment thereof is subcutaneously administered to the subject. In some embodiments, the anti-NPR1 antibody or antigen binding fragment thereof is administered about once every week, once every 2 weeks, about once every 3 weeks, about once every 4 weeks, about once every 5 weeks, about once every 6 weeks, about once every 7 weeks, about once every 8 weeks, about once every 9 weeks, about once every 10 weeks, about once every 11 weeks, or about once every 12 weeks. In some embodiments, the anti-NPR1 antibody or antigen binding fragment thereof is administered about once every week. In some embodiments, the anti-NPR1 antibody or antigen binding fragment thereof is administered about once every 2 weeks. In some embodiments, the anti-NPR1 antibody or antigen binding fragment thereof is administered about once every 3 weeks. In some embodiments, the anti-NPR1 antibody or antigen binding fragment thereof is administered about once every 4 weeks. In some embodiments, the anti-NPR1 antibody or antigen binding fragment thereof is administered about once every month. In some embodiments, the anti-NPR1 antibody or antigen binding fragment thereof is administered about once every 5 weeks. In some embodiments, the anti-NPR1 antibody or antigen binding fragment thereof is administered about once every 6 weeks. In some embodiments, the anti-NPR1 antibody or antigen binding fragment thereof is administered about once every 7 weeks. In some embodiments, the anti-NPR1 antibody or antigen binding fragment thereof is administered about once every 8 weeks. In some embodiments, the anti-NPR1 antibody or antigen binding fragment thereof is administered about once every two months. In some embodiments, the anti-NPR1 antibody or antigen binding fragment thereof is provided for administration about once every 9 weeks. In some embodiments, the anti-NPR1 antibody or antigen binding fragment thereof is provided for administration about once every 10 weeks. In some embodiments, the anti-NPR1 antibody or antigen binding fragment thereof is provided for administration about once every 11 weeks. In some embodiments, the anti-NPR1 antibody or antigen binding fragment thereof is provided for administration about once every 12 weeks. In some embodiments, the anti-NPR1 antibody or antigen binding fragment thereof is provided for administration about once every three months. In some embodiments, the anti-NPR1 antibody or antigen binding fragment thereof is provided in a form for subcutaneous administration. In some embodiments, the anti-NPR1 antibody or antigen binding fragment thereof is provided for administration about once every week, once every 2 weeks, about once every 3 weeks, about once every 4 weeks, about once every 5 weeks, about once every 6 weeks, about once every 7 weeks, about once every 8 weeks, about once every 9 weeks, about once every 10 weeks, about once every 11 weeks, or about once every 12 weeks. In some embodiments, the anti-NPR1 antibody or antigen binding fragment thereof is provided for administration about once every week. In some embodiments, the anti-NPR1 antibody or antigen binding fragment thereof is provided for administration about once every 2 weeks. In some embodiments, the anti-NPR1 antibody or antigen binding fragment thereof is provided for administration about once every 3 weeks. In some embodiments, the anti-NPR1 antibody or antigen binding fragment thereof is provided for administration about once every 4 weeks. In some embodiments, the anti-NPR1 antibody or antigen binding fragment thereof is provided for administration about once every month. In some embodiments, the anti-NPR1 antibody or antigen binding fragment thereof is provided for administration about once every 5 weeks. In some embodiments, the anti-NPR1 antibody or antigen binding fragment thereof is provided for administration about once every 6 weeks. In some embodiments, the anti-NPR1 antibody or antigen binding fragment thereof is provided for administration about once every 7 weeks. In some embodiments, the anti-NPR1 antibody or antigen binding fragment thereof is provided for administration about once every 8 weeks. In some embodiments, the anti-NPR1 antibody or antigen binding fragment thereof is provided for administration about once every two months. In some embodiments, the anti-NPR1 antibody or antigen binding fragment thereof is provided for administration about once every 9 weeks. In some embodiments, the anti-NPR1 antibody or antigen binding fragment thereof is provided for administration about once every 10 weeks. In some embodiments, the anti-NPR1 antibody or antigen binding fragment thereof is provided for administration about once every 11 weeks. In some embodiments, the anti-NPR1 antibody or antigen binding fragment thereof is provided for administration about once every 12 weeks. In some embodiments, the anti-NPR1 antibody or antigen binding fragment thereof is provided for administration about once every three months.

### BRIEF DESCRIPTION OF THE FIGURES

FIG. 1: A graphical representation of a first-in-human (FIH) single ascending dose (SAD) study to explore safety, tolerability, and pharmacokinetics in healthy participants.

FIG. 2: A chart containing graphics and baseline characteristics of participants in the FIH SAD study for which interim analysis data are presented.

FIG. 3: A chart containing results regarding safety and tolerability in participants of non-Japanese descent in the FIH SAD study for which interim analysis data are presented. N=number of participants; nE=number of AEs in the category; nS=number of participants with at least one AE in the category; % is based on the number of participants.

FIG. 4: A chart containing results regarding safety and tolerability in participants of Japanese descent in the FIH SAD study for which interim analysis data are presented. N=number of participants; nE=number of AEs in the category; nS=number of participants with at least one AE in the category; % is based on the number of participants.

FIG. 5: A graphical representation of the serum concentrations of the anti-NPR1 antibody after a single subcutaneous dose of 1 to 240 mg for the non-Japanese descent cohort and the Japanese descent 240 mg dose cohort. The Tmax was determined to be 4˜10 days and the half-life (T½) at 120 and 240 mg was determined to be 15-23 days.

FIG. 6: A graphical representation of the plasma cGMP levels (ng/mL) from the interim analysis results for the non-Japanese descent cohorts over time after a single subcutaneous administration of the anti-NPR1 antibody at 30 mg, 60 mg, 120 mg, and 240 mg in comparison with placebo results.

FIG. 7: A graphical representation of the 24 h ABPM SBP (systolic blood pressure) CFB (mmHG) from the interim analysis results for the non-Japanese descent cohorts over time after a single subcutaneous administration of the anti-NPR1 antibody at 30 mg, 60 mg, 120 mg, and 240 mg in comparison with placebo results.

FIG. 8: A graphical representation of the 24 h ABPM DBP (diastolic blood pressure) CFB (mmHG) from the interim analysis results for the non-Japanese descent cohorts over time after a single subcutaneous administration of the anti-NPR1 antibody at 30 mg, 60 mg, 120 mg, and 240 mg in comparison with placebo results.

FIG. 9: A graphical representation of the 24 h ABPM heart rate (HR) CFB (beats per minute; bpm) from the interim analysis results for the non-Japanese descent cohorts over time after a single subcutaneous administration of the anti-NPR1 antibody at 30 mg, 60 mg, 120 mg, and 240 mg in comparison with placebo results.

FIG. 10: A graphical representation of Cohort 1 of a safety and tolerability study in heart failure participants with Reduced Ejection Fraction (HFrEF) or mildly reduced ejection fraction (HFmrEF).

FIG. 11: A graphical representation of Cohort 2 of a safety and tolerability study in heart failure participants with Reduced Ejection Fraction (HFrEF) or mildly reduced ejection fraction (HFmrEF).

FIG. 12: A graphical representation of an efficacy, safety, tolerability and dose finding study of an anti-NPR-1 antibody in resistant hypertension patients. ABPM=ambulatory blood pressure monitoring; mean 24 hr SBP=mean ambulatory systolic blood pressure (based on 24 hr ambulatory blood pressure monitoring); msSBP=mean sitting systolic blood pressure (based on office assessment); SC=subcutaneously; WOCBP=women of childbearing potential.

### GENERAL DEFINITIONS

In order that the methods and uses described herein may be more readily understood, certain terms are first defined. Additional definitions are set forth throughout the detailed description as required.

Throughout the description and claims of this specification, the words “comprise” and “contain” and variations of the words, for example “comprising” and “comprises”, mean “including but not limited to”, and do not exclude other components, integers, or steps. Moreover the singular encompasses the plural unless the context otherwise requires: in particular, where the indefinite article is used, the specification is to be understood as contemplating plurality as well as singularity, unless the context requires otherwise.

As used herein, “NPR1” and “NPR1 protein” refers to Natriuretic Peptide Receptor 1. This protein is also known as Atrial natriuretic peptide receptor type A (ANP-A, ANPR-A or NPR-A) and Guanylate cyclase A (GC-A). In some embodiments, the NPR1 referred to is human NPR1. In some embodiments the human NPR1 is has UniProt accession number P16066 or GenBank Accession number EAW53284.1 (SEQ ID NO: 1). In some embodiments, the NPR1 referred to is mouse (Mus musculus) NPR1. In some embodiments the mouse NPR1 has NCBI Reference Sequence number NP_032753.5 (SEQ ID NO: 2). In some embodiments, the NPR1 referred to is rat (Rattus norvegicus) NPR1. In some embodiments the rat NPR1 has NCBI Reference Sequence number NP_036745.1 (SEQ ID NO: 3). Exemplary NPR1 proteins are shown in Table 1.

TABLE 1
NPR1 Protein Sequences

| SEQ ID NO: | Animal | Sequence (amino acid) |
|---|---|---|
| 1 | Human | MPGPRRPAGSRLRLLLLLLLPPLLLLLRGSHAGNLTVAVVLPLANTSYPWS |
| | | WARVGPAVELALAQVKARPDLLPGWTVRTVLGSSENALGVCSDTAAPLA |
| | | AVDLKWEHNPAVFLGPGCVYAAAPVGRFTAHWRVPLLTAGAPALGFGV |
| | | KDEYALTTRAGPSYAKLGDFVAALHRRLGWERQALMLYAYRPGDEEHC |
| | | FFLVEGLFMRVRDRLNITVDHLEFAEDDLSHYTRLLRTMPRKGRVIYICSS |
| | | PDAFRTLMLLALEAGLCGEDYVFFHLDIFGQSLQGGQGPAPRRPWERGDG |
| | | QDVSARQAFQAAKIITYKDPDNPEYLEFLKQLKHLAYEQFNFTMEDGLVN |
| | | TIPASFHDGLLLYIQAVTETLAHGGTVTDGENITQRMWNRSFQGVTGYLK |
| | | IDSSGDRETDFSLWDMDPENGAFRVVLNYNGTSQELVAVSGRKLNWPLG |
| | | YPPPDIPKCGFDNEDPACNQDHLSTLEVLALVGSLSLLGILIVSFFIYRKMQ |
| | | LEKELASELWRVRWEDVEPSSLERHLRSAGSRLTLSGRGSNYGSLLTTEG |
| | | QFQVFAKTAYYKGNLVAVKRVNRKRIELTRKVLFELKHMRDVQNEHLTR |
| | | FVGACTDPPNICILTEYCPRGSLQDILENESITLDWMFRYSLTNDIVKGMLF |
| | | LHNGAICSHGNLKSSNCVVDGRFVLKITDYGLESFRDLDPEQGHTVYAKK |
| | | LWTAPELLRMASPPVRGSQAGDVYSFGIILQEIALRSGVFHVEGLDLSPKEI |
| | | IERVTRGEQPPFRPSLALQSHLEELGLLMQRCWAEDPQERPPFQQIRLTLR |
| | | KFNRENSSNILDNLLSRMEQYANNLEELVEERTQAYLEEKRKAEALLYQI |
| | | LPHSVAEQLKRGETVQAEAFDSVTIYFSDIVGFTALSAESTPMQVVTLLND |
| | | LYTCFDAVIDNFDVYKVETIGDAYMVVSGLPVRNGRLHACEVARMALAL |
| | | LDAVRSFRIRHRPQEQLRLRIGIHTGPVCAGVVGLKMPRYCLFGDTVNTA |
| | | SRMESNGEALKIHLSSETKAVLEEFGGFELELRGDVEMKGKGKVRTYWL |
| | | LGERGSSTRG |
| 2 | Mouse | MPGSRRVRPRLRALLLLPPLLLLRSGHASDLTVAVVLPLTNTSYPWSWAR |
| | | VGPAVELALGRVKARPDLLPGWTVRMVLGSSENAAGVCSDTAAPLAAV |
| | | DLKWEHSPAVFLGPGCVYSAAPVGRFTAHWRVPLLTAGAPALGIGVKDE |
| | | YALTTRTGPSHVKLGDFVTALHRRLGWEHQALVLYADRLGDDRPCFFIV |
| | | EGLYMRVRERLNITVNHQEFVEGDPDHYTKLLRTVQRKGRVIYICSSPDA |
| | | FRNLMLLALDAGLTGEDYVFFHLDVFGQSLQGAQGPVPRKPWERDDGQ |
| | | DRRARQAFQAAKIITYKEPDNPEYLEFLKQLKLLADKKFNFTMEDGLKNII |
| | | PASFHDGLLLYVQAVTETLAQGGTVTDGENITQRMWNRSFQGVTGYLKI |
| | | DRNGDRDTDFSLWDMDPETGAFRVVLNFNGTSQELMAVSEHRLYWPLG |
| | | YPPPDIPKCGFDNEDPACNQDHFSTLEVLALVGSLSLVSFLIVSFFIYRKMQ |
| | | LEKELVSELWRVRWEDLQPSSLERHLRSAGSRLTLSGRGSNYGSLLTTEG |
| | | QFQVFAKTAYYKGNLVAVKRVNRKRIELTRKVLFELKHMRDVQNEHLTR |
| | | FVGACTDPPNICILTEYCPRGSLQDILENESITLDWMFRYSLTNDIVKGMLF |
| | | LHNGAIGSHGNLKSSNCVVDGRFVLKITDYGLESFRDPEPEQGHTLFAKK |
| | | LWTAPELLRMASPPARGSQAGDVYSFGIILQEIALRSGVFYVEGLDLSPKEI |
| | | IERVTRGEQPPFRPSMDLQSHLEELGQLMQRCWAEDPQERPPFQQIRLALR |
| | | KFNKENSSNILDNLLSRMEQYANNLEELVEERTQAYLEEKRKAEALLYQI |
| | | LPHSVAEQLKRGETVQAEAFDSVTIYFSDIVGFTALSAESTPMQVVTLLND |
| | | LYTCFDAVIDNFDVYKVETIGDAYMVVSGLPVRNGQLHAREVARMALAL |
| | | LDAVRSFRIRHRPQEQLRLRIGIHTGPVCAGVVGLKMPRYCLFGDTVNTA |
| | | SRMESNGEALRIHLSSETKAVLEEFDGFELELRGDVEMKGKGKVRTYWL |
| | | LGERGCSTRG |
| 3 | Rat | MPGSRRVRPRLRALLLLPPLLLLRGGHASDLTVAVVLPLTNTSYPWSWAR |
| | | VGPAVELALARVKARPDLLPGWTVRMVLGSSENAAGVCSDTAAPLAAV |
| | | DLKWEHSPAVFLGPGCVYSAAPVGRFTAHWRVPLLTAGAPALGIGVKDE |
| | | YALTTRTGPSHVKLGDFVTALHRRLGWEHQALVLYADRLGDDRPCFFIV |
| | | EGLYMRVRERLNITVNHQEFVEGDPDHYPKLLRAVRRKGRVIYICSSPDA |
| | | FRNLMLLALNAGLTGEDYVFFHLDVFGQSLKSAQGLVPQKPWERGDGQD |
| | | RSARQAFQAAKIITYKEPDNPEYLEFLKQLKLLADKKFNFTVEDGLKNIIP |
| | | ASFHDGLLLYVQAVTETLAQGGTVTDGENITQRMWNRSFQGVTGYLKID |
| | | RNGDRDTDFSLWDMDPETGAFRVVLNYNGTSQELMAVSEHKLYWPLGY |
| | | PPPDVPKCGFDNEDPACNQDHFSTLEVLALVGSLSLISFLIVSFFIYRKMQL |
| | | EKELVSELWRVRWEDLQPSSLERHLRSAGSRLTLSGRGSNYGSLLTTEGQ |
| | | FQVFAKTAYYKGNLVAVKRVNRKRIELTRKVLFELKHMRDVQNEHLTRF |
| | | VGACTDPPNICILTEYCPRGSLQDILENESITLDWMFRYSLTNDIVKGMLFL |
| | | HNGAICSHGNLKSSNCVVDGRFVLKITDYGLESFRDPEPEQGHTLFAKKL |
| | | WTAPELLRMASPPARGSQAGDVYSFGIILQEIALRSGVFYVEGLDLSPKEII |
| | | ERVTRGEQPPFRPSMDLQSHLEELGQLMQRCWAEDPQERPPFQQIRLALR |
| | | KFNKENSSNILDNLLSRMEQYANNLEELVEERTQAYLEEKRKAEALLYQI |
| | | LPHSVAEQLKRGETVQAEAFDSVTIYFSDIVGFTALSAESTPMQVVTLLND |
| | | LYTCFDAVIDNFDVYKVETIGDAYMVVSGLPVRNGQLHAREVARMALAL |
| | | LDAVRSFRIRHRPQEQLRLRIGIHTGPVCAGVVGLKMPRYCLFGDTVNTA |
| | | SRMESNGEALKIHLSSETKAVLEEFDGFELELRGDVEMKGKGKVRTYWL |
| | | LGERGCSTRG |

In various embodiments, the anti-NPR1 antibodies and antigen binding fragments described herein are capable of binding to NPR1 and activating NPR1 in the absence of ANP. By virtue of this activity, the described anti-NPR1 antibodies and antigen binding fragments may be useful in treating undesirable conditions, diseases and disorders including cardiovascular disorders (e.g., hypertension, peripheral vascular disease, heart failure (including but not limited to heart failure with reduced ejection fraction (HFrEF), heart failure with mid-range ejection fraction or heart failure with mildly reduced ejection fraction or heart failure with mildly reduced ejection fraction (HFmrEF), heart failure with preserved ejection fraction (HFpEF), heart failure after acute myocardial infarct, or acute decompensated heart failure), coronary artery disease (CAD), ischemic heart disease (IHD), mitral stenosis and regurgitation, angina, hypertrophic cardiomyopathy (e.g., ventricular hypertrophy), diabetic cardiomyopathy, supraventricular and ventricular arrhythmias, cardiac dysrhythmia, atrial fibrillation (AF), new onset of atrial fibrillation, recurrent atrial fibrillation, cardiac fibrosis, atrial flutter, detrimental vascular remodeling, plaque stabilization, or myocardial infarction (MI)), hypertension (e.g., resistant hypertension, hypertensive heart disease, pulmonary hypertension, isolated systolic hypertension, or pulmonary arterial hypertension), preeclampsia, asthma, glaucoma, cytokine release syndrome, and/or a kidney disorder (e.g., diabetic renal insufficiency, non-diabetic renal insufficiency, renal failure, diabetic nephropathy, non-diabetic nephropathy, acute renal injury, contrast induced nephropathy, nephrotic syndrome, glomerulonephritis, scleroderma, glomerular sclerosis, proteinuria of primary renal disease, renal vascular hypertension, diabetic retinopathy and end-stage renal disease (ESRD), endothelial dysfunction, diastolic dysfunction, renal fibrosis, and polycystic kidney disease (PKD)) as further described herein.

The term “antibody” as used herein refers to a whole antibody or antigen binding fragment thereof. A whole antibody is a glycoprotein comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system. The term “antibody” includes, but is not limited to, monoclonal antibodies, human antibodies, humanized antibodies, camelised antibodies, and chimeric antibodies. The antibodies can be of any isotype/class (e.g., IgG, IgE, IgM, IgD, IgA and IgY) or subclass (e.g., IgG1, IgG2, IgG3, IgG4, IgA1, and IgA2).

The term “antigen binding fragment” refers to a fragment of an intact antibody that retains the ability to specifically bind to a given antigen (e.g., NPR1) and/or provide a function of the intact antibody. Such fragments include Fab fragments, Fab′ fragments, monovalent fragments consisting of the VL, VH, CL and CH1 domains; F(ab′)2 fragments, bivalent fragments comprising two Fab fragments linked by a disulfide bridge at the hinge region; an Fd fragment consisting of the VH and CH1 domains; an Fv fragment consisting of the VL and VH domains, a single chain Fv fragment (scFv) consisting of the VL and VH domains connected by a linker sequence; and a single domain antibody (dAb) fragment (Ward et al., 1989 Nature 341:544-546), which consists of a VH domain or a VL domain.

The term “single chain antibody”, “single chain Fv” or “scFv” is refers to a molecule comprising an antibody heavy chain variable domain (or region; VH) and an antibody light chain variable domain (or region; VL) connected by a linker. Such scFv molecules can have the general structures: NH2-VL-linker-VH-COOH or NH2-VH-linker-VL-COOH. Any suitable linker may be used. A non-limiting set of linkers that can be used in such single chain antibodies are described by Holliger et al. (1993), Proc. Natl. Acad. Sci. USA 90:6444-6448, Alfthan et al. (1995), Protein Eng. 8:725-731, Choi et al. (2001), Eur. J. Immunol. 31:94-106, Hu et al. (1996), Cancer Res. 56:3055-3061, Kipriyanov et al. (1999), J. Mol. Biol. 293:41-56 and Roovers et al. (2001), Cancer Immunol; the contents of each of which are herein incorporated by reference for this purpose. Such single chain antibodies are also intended to be encompassed within the term “antigen binding fragment” of an antibody. These antibody fragments are obtained using techniques known to those of skill in the art, and the fragments are screened for utility in the same manner as are intact antibodies. Without limitation, an antigen binding fragment can be produced by any suitable known method. For instance, the various antigen binding fragments described herein can be produced by enzymatic or chemical modification of intact antibodies, synthesized de novo using recombinant DNA methodologies (e.g., single chain Fv), or identified using phage display libraries (see, e.g., Pini and Bracci, Curr Protein Pept Sci 2000; 1(2):155-69, the contents of which are herein incorporated by reference for this purpose). Antigen binding fragments are screened for utility (e.g., binding affinity, activity) in the same manner as are intact antibodies.

Antigen binding fragments can also be incorporated into single domain antibodies, maxibodies, minibodies, intrabodies, diabodies, triabodies, tetrabodies, v-NAR and bis-scFv (see, e.g., Hollinger and Hudson, 2005, Nature Biotechnology, 23, 9, 1126-1136, the contents of which are herein incorporated by reference for this purpose). Antigen binding portions of antibodies can be grafted into scaffolds based on polypeptides such as Fibronectin type III (Fn3) (see e.g., U.S. Pat. No. 6,703,199, which describes fibronectin polypeptide monobodies, the contents of which are herein incorporated by reference for this purpose).

Antigen binding fragments can be incorporated into single chain molecules comprising a pair of tandem Fv segments (VH-CH1-VH-CH1) which, together with complementary light chain polypeptides, form a pair of antigen binding regions (see Zapata et al., 1995 Protein Eng. 8(10):1057-1062; and U.S. Pat. No. 5,641,870; the contents of each of which are herein incorporated by reference for this purpose).

The terms “individual”, “subject”, “host”, and “patient” are used interchangeably herein and refer to any human subject for whom diagnosis, treatment, or therapy is desired.

As used herein, “individual in need thereof”, “subject in need thereof”, and “patient in need thereof” refers to a human selected for treatment or therapy that is in need of such treatment or therapy (e.g., has one or more of the diseases or disorders described herein).

The term “isolated” means throughout this specification, that the immunoglobulin, antibody or polynucleotide, as the case may be, exists in a physical milieu distinct from that in which it may occur in nature. For example, a naturally-occurring polynucleotide or polypeptide present in a living organism is not isolated, but the same polynucleotide or polypeptide separated from some or all of the coexisting materials in the living organism, is isolated.

Throughout this specification, complementarity determining regions (“CDR”) are defined according to the Kabat definition unless specified that the CDR are defined according to another definition. The precise amino acid sequence boundaries of a given CDR can be determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273, 927-948 (“Chothia” numbering scheme) and ImMunoGenTics (IMGT) numbering (Lefranc, M.-P., The Immunologist, 7, 132-136 (1999); Lefranc, M.-P. et al., Dev. Comp. Immunol., 27, 55-77 (2003) (“IMGT” numbering scheme); the contents of each of which are herein incorporated by reference for this purpose. For example, for classic formats, under Kabat, the CDR amino acid residues in the heavy chain variable domain (VH) are numbered 31-35 (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3). Under Chothia the CDR amino acids in the VH are numbered 26-32 (HCDR1), 52-56 (HCDR2), and 95-102 (HCDR3); and the amino acid residues in VL are numbered 26-32 (LCDR1), 50-52 (LCDR2), and 91-96 (LCDR3). By combining the CDR definitions of both Kabat and Chothia, the CDRs consist of amino acid residues 26-35 (HCDR1), 50-65 (HCDR2), and 95-102 (HCDR3) in human VH and amino acid residues 24-34 (LCDR1), 50-56 (LCDR2), and 89-97 (LCDR3) in human VL. Under IMGT the CDR amino acid residues in the VH are numbered approximately 26-35 (CDR1), 51-57 (CDR2) and 93-102 (CDR3), and the CDR amino acid residues in the VL are numbered approximately 27-32 (CDR1), 50-52 (CDR2), and 89-97 (CDR3) (numbering according to “Kabat”). Under IMGT, the CDR regions of an antibody can be determined using the program IMGT/DomainGap Align.

By convention, the CDR regions in the heavy chain are typically referred to as HCDR1, HCDR2 and HCDR3 and in the light chain as LCDR1, LCDR2 and LCDR3. They are numbered sequentially in the direction from the amino terminus to the carboxy terminus.

The term “antibody framework” as used herein refers to the part of the variable domain, either VL or VH, which serves as a scaffold for the antigen binding loops (CDRs) of this variable domain. In essence, it is the variable domain without the CDRs.

The terms “constant region” or “constant domain” refer to a carboxy terminal portion of the light and heavy chain which is not directly involved in binding of the antibody to antigen but exhibits various effector functions, such as interaction with the Fc receptor. The terms refer to the portion of an immunoglobulin molecule having a more conserved amino acid sequence relative to the other portion of the immunoglobulin, the variable domain, which contains the antigen binding site. The constant domain contains the CH1, CH2 and CH3 domains of the heavy chain and the CHL domain of the light chain.

The term “epitope” or “antigenic determinant” refers to a site on an antigen to which an immunoglobulin or antibody specifically binds (e.g., a specific site on the target molecule). An epitope typically includes at least 3, 4, 5, 6, 7, 8, 9, 10, 11, 12, 13, 14, or 15 consecutive or non-consecutive amino acids in a unique spatial conformation. See, e.g., Epitope Mapping Protocols in Methods in Molecular Biology, Vol. 66, G. E. Morris, Ed. (1996), the contents of which are herein incorporated by reference for this purpose. In addition, as used herein, an epitope can comprise one or more monosaccharide units of a polysaccharide to which an antibody specifically binds. In specific aspects, an epitope can be a conformational epitope. See, e.g., Thompson et al., 2009, J. of Biol. Chem. 51:35621-35631, the contents of which are herein incorporated by reference for this purpose.

The terms “monoclonal antibody” or “monoclonal antibody composition” as used herein refers to an antibody obtained from a population of substantially homogeneous antibodies produced by a particular cell or cell line, wherein the individual antibodies comprising the population are essentially identical in sequence except for possible naturally-occurring mutations that may be present in minor amounts. A monoclonal antibody preparation displays a single binding specificity and affinity for a particular epitope. In contrast, conventional (polyclonal) antibody preparations typically include a multitude of antibodies directed against or specific for different epitopes. The modifier “monoclonal” indicates the character of the antibody as being obtained from a substantially homogeneous population of antibodies and is not to be construed as requiring production of the antibody by any particular method. Monoclonal antibodies (mAbs) can be produced by a variety of techniques, including conventional methodology, e.g., the standard somatic cell hybridization technique of Kohler and Milstein (Nature 1975; 256(5517):495-7), the contents of which are herein incorporated by reference for this purpose. A monoclonal antibody may also be obtained from other suitable methods, including phage display techniques such as those described in Clackson et al. (Nature 1991; 352(6336):624-8) or Marks et al. (J Mol Biol 1991; 222(3):581-97), the contents of each of which are herein incorporated by reference for this purpose. The term “monoclonal antibody” is also not limited to antibody sequences from particular species of origin or from one single species of origin. Thus, the meaning of the term “monoclonal antibody” encompasses chimeric monoclonal antibodies such as humanized monoclonal antibodies.

The term “chimeric antibody,” as used herein, refers to antibodies in which (a) the constant region is altered, replaced, or exchanged such that the antigen binding site (variable region) is linked to a constant region of a different or altered class, effector function, and/or species; or (b) the variable region, or a portion thereof, is altered, replaced, or exchanged with a variable region, or a portion thereof, having a different or altered antigen specificity. To create a chimeric antibody, the variable region sequences from a non-human donor antibody (e.g., a mouse, rabbit, or rat donor antibody) can be linked to human constant regions using methods known in the art (see, e.g., U.S. Pat. No. 4,816,567 (Cabilly et al.), the contents of which are herein incorporated by reference for this purpose). For instance, a mouse anti-NPR1 antibody can be modified by replacing its constant region with the constant region from a human immunoglobulin. Due to the replacement with a human constant region, the chimeric antibody can retain its specificity in recognizing human NPR1 while having reduced immunogenicity in human as compared to the original mouse antibody.

As used herein, the term “humanized antibody” refers to forms of antibodies that contain at least some human sequence and at least some non-human sequence. Typically, the antibody contains human sequences and a minor portion of non-human sequences which confer binding specificity to the target antigen. Such antibodies are chimeric antibodies which contain minimal sequence derived from a non-human immunoglobulin and retain the reactivity of a non-human antibody while being less immunogenic in humans. Typically, humanized antibodies are generated by replacing hypervariable region sequences from a human acceptor antibody with hypervariable region sequences from a non-human donor antibody (e.g., a mouse, rabbit, or rat donor antibody) that binds to an antigen of interest (e.g., NPR1). In some cases, framework region sequences of the acceptor antibody may also be replaced with the corresponding sequences of the donor antibody (e.g., via affinity maturation). In addition to the sequences derived from the donor and acceptor antibodies, the humanized antibody can be further modified by the substitution of residues, either in the framework region and/or within the replaced non-human residues to refine and optimize antibody specificity, affinity, and/or activity, as discussed herein. Methods to generate humanized antibodies are known in the art. See, e.g., Riechmann et al. (Nature 1988; 332(6162):323-7); Jones et al. (Nature 1986; 321(6069):522-5); U.S. Pat. No. 5,225,539 (Winter); and U.S. Pat. Nos. 5,530,101; 5,585,089; 5,693,762, and 6,180,370 (Queen et al.), the contents of each of which are herein incorporated by reference for this purpose.

The term “human antibody”, as used herein, is intended to include antibodies having variable regions in which both the framework and CDR regions are derived from sequences of human origin. Furthermore, if the antibody contains a constant region, the constant region also is derived from such human sequences, e.g., human germline sequences, or mutated versions of human germline sequences or antibody containing consensus framework sequences derived from human framework sequences analysis, for example, as described in Knappik, et al., (2000) J Mol Biol; 296:57-86, the contents of which are herein incorporated by reference for this purpose). Human antibodies may include amino acid residues not encoded by human sequences (e.g., mutations introduced by random or site-specific mutagenesis in vitro or by somatic mutation in vivo).

The antibodies or antigen binding fragments described herein may include amino acid residues not encoded by human sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences.

The terms “peptide,” “polypeptide,” and “protein” are used interchangeably herein to refer to a polymer of amino acid residues. The terms encompass amino acid polymers in which one or more amino acid residues is an artificial chemical mimetic of a corresponding naturally-occurring amino acid, as well as naturally-occurring amino acid polymers and non-naturally-occurring amino acid polymers. Unless otherwise indicated, a particular polypeptide sequence also implicitly encompasses conservatively modified variants thereof.

The term “conservatively modified variant” applies to both amino acid and nucleic acid sequences. For nucleic acid sequences, conservatively modified variants refer to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG, and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence. For polypeptide sequences, conservatively modified variants include individual substitutions, deletions, or additions to a polypeptide sequence which result in the substitution of an amino acid with a chemically similar amino acid. Conservative substitution tables providing functionally similar amino acids are well known in the art. The following eight groups contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine(S), Threonine (T); and
8) Cysteine (C), Methionine (M).

The term “identity” or “homology” refers to a relationship between the sequences of two or more polypeptides, as determined by comparing the sequences. “Identity” also means the degree of sequence relatedness between polypeptides, as determined by the number of matches between strings of two or more amino acid residues. The percent “identity” between the two sequences is a function of the number of identical positions shared by the sequences (i.e., percent identity equals number of identical positions/total number of positions×100), taking into account the number of gaps, and the length of each gap, which need to be introduced for optimal alignment of the two sequences. The comparison of sequences and determination of percent identity between two sequences can be accomplished using a mathematical algorithm. For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. Additionally, or alternatively, the protein sequences described herein can further be used as a “query sequence” to perform a search against public databases to, for example, identify related sequences. For example, such searches can be performed using the BLAST program of Altschul et al. (J Mol Biol 1990; 215(3):403-10), the contents of which are herein incorporated by reference for this purpose.

Two sequences are “substantially identical” if two sequences have a specified percentage of amino acid residues or nucleotides that are the same (e.g., 60% identity, 65% identity, 70% identity, 75% identity, 80% identity, 85% identity, 90% identity, 95% identity, or 99% identity over a specified region, or, when not specified, over the entire sequence), when compared and aligned for maximum correspondence over a comparison window, or designated region as measured using one of the following sequence comparison algorithms or by manual alignment and visual inspection. Optionally, the identity exists over a region that is at least about 50 nucleotides (or 10 amino acids) in length, or exists over a region that is 100 to 500 or 1000 or more nucleotides (or 20, 50, 200 or more amino acids) in length.

Binding “affinity” refers to the strength of interaction between antibody and antigen at single antigenic sites. Within each antigenic site, the variable region of the antibody “arm” interacts through weak non-covalent forces with antigen at numerous sites. In general, the more interactions, the stronger the affinity. Generally, such determinations can be made using a cell-based assay.

The term “Kassoc” or “Ka”, as used herein, is intended to refer to the association rate of a particular binding molecule-antigen interaction, whereas the term “Kdis” or “Kd,” as used herein, is intended to refer to the dissociation rate of a particular binding molecule-antigen interaction. The term “K_{D}”, as used herein, is intended to refer to the equilibrium dissociation constant, which is obtained from the ratio of Kd to Ka (i.e., Kd/Ka) and is expressed as a molar concentration (M). K_{D} values for antibodies can be determined using methods well established in the art. A method for determining the K_{D} of an antibody is by using surface plasmon resonance, such as a Biacore® system, or solution equilibrium titration (SET) (see Friguet et al., (1985) J. Immunol. Methods, 77(2):305-319, and Hanel et al., (2005) Anal. Biochem., 339(1):182-184), the contents of each of which are herein incorporated by reference for this purpose.

As used herein, the term “specific,” “specifically binds,” and “binds specifically” refers to a binding reaction between an antibody or antigen binding fragment (e.g., an anti-NPR1 antibody) and a target antigen (e.g., NPR1) in a heterogeneous population of proteins and other biologics. Antibodies can be tested for specificity of binding by comparing binding to an appropriate antigen to binding to an irrelevant antigen or antigen mixture under a given set of conditions. If the antibody binds to the appropriate antigen with at least 2, 5, 7, and preferably 10 or more times more affinity than to the irrelevant antigen or antigen mixture, then it is considered to be specific. A “specific antibody” or a “target-specific antibody” is one that only binds the target antigen (e.g., NPR1), but does not bind (or exhibits minimal binding) to other antigens. In certain embodiments, an antibody or antigen binding fragment that specifically binds the target antigen (e.g., NPR1) has a K_{D} of less than 1×10⁻⁶ M, less than 1×10⁻⁷ M, less than 1×10⁻⁸ M, less than 1×10⁻⁹ M, less than 1×10⁻¹⁰ M, less than 1×10⁻¹¹ M, less than 1×10⁻¹² M, or less than 1×10⁻¹³ M. In certain embodiments, the K_{D} is about 1 pM to about 600 pM. In certain embodiments, the K_{D} is between 600 pM to 1 μM, 1 μM to 100 nM, or 100 mM to 10 nM (inclusive).

The term “about” in relation to a numerical value x means, for example, x±10%. In certain embodiments, the term “about” in relation to a numerical value x means, for example, x±9%, x±8%, x±7%, x±6%, x±5%, x±4%, x±3%, x±2%, or x±1%.

### Antibodies and Antigen Binding Fragments

Below are described certain specific anti-NPR1 antibody sequences for use in the methods and uses described herein. As used herein, the term “anti-NPR1 antibody” or “antibody that binds to NPR1” refers to any form of an antibody or antigen binding fragment that specifically binds to NPR1, e.g., those binding with a K_{D} of less than 1×10⁻⁸M, as determined by, e.g., surface plasmon resonance (SPR) spectroscopy (using Biacore™) or solution equilibrium titration (SET). The term encompasses monoclonal antibodies (including intact monoclonal antibodies), polyclonal antibodies, and biologically functional antigen binding fragments so long as they specifically bind to NPR1.

Amino acid and nucleic acid sequences of exemplary anti-NPR1 antibodies presented herein are set forth in Table 2. In some embodiments, the antibody has the heavy and light chain CDRs, VH and VL sequence, and/or the heavy and light chain sequence of any of the antibodies described in Table 2. In some embodiments, the anti-NPR1 antibody is a four-chain antibody (also referred to as an intact antibody), comprising two heavy chains and two light chains. In some embodiments, the anti-NPR1 antibody is an antigen binding fragment of an intact antibody, e.g., a functional fragment of an intact antibody selected from any of those set forth in Table 2 that retains the ability to bind NPR1 and/or provide a function of the intact antibody (e.g., activating NPR1 in the absence of ANP). In some embodiments, the anti-NPR1 antibody is an antibody having the CDRs of any heavy chain variable region and light chain variable region pair shown in Table 2. In some embodiments, the anti-NPR1 antibody is an antibody having the CDRs of any heavy and light chain pair shown in Table 2.

TABLE 2
Exemplary anti-NPR1 antibody sequences
N001_DAPA

| | | |
|---|---|---|
| SEQ ID NO: 4 | HCDR1 (Combined) | GFTFSSYWMN |
| SEQ ID NO: 5 | HCDR2 (Combined) | VIESKGNYIFYADSVKG |
| SEQ ID NO: 6 | HCDR3 (Combined) | DRYSMIYSYGAGAFDY |
| SEQ ID NO: 7 | HCDR1 (Kabat) | SYWMN |
| SEQ ID NO: 5 | HCDR2 (Kabat) | VIESKGNYIFYADSVKG |
| SEQ ID NO: 6 | HCDR3 (Kabat) | DRYSMIYSYGAGAFDY |
| SEQ ID NO: 8 | HCDR1 (Chothia) | GFTFSSY |
| SEQ ID NO: 9 | HCDR2 (Chothia) | ESKGNY |
| SEQ ID NO: 6 | HCDR3 (Chothia) | DRYSMIYSYGAGAFDY |
| SEQ ID NO: 10 | HCDR1 (IMGT) | GFTFSSYW |
| SEQ ID NO: 11 | HCDR2 (IMGT) | IESKGNYI |
| SEQ ID NO: 12 | HCDR3 (IMGT) | ARDRYSMIYSYGAGAFDY |
| SEQ ID NO: 13 | VH | EVQLLESGGGLVQPGGSLRLSCAASGFTFSSYWMNWVRQAPGKGLEWVS |
| | | VIESKGNYIFYADSVKGRFTISRDNSKNTLYLQMNSLRAEDTAVYYCARDR |
| | | YSMIYSYGAGAFDYWGQGTLVTVSS |
| SEQ ID NO: 14 | DNA VH | gaagtgcagctgctggaatccggcggaggtctggtccagcctggaggttccctgcgcctgtcatgcgcagcctccgg |
| | | attcaccttttcgtcgtactggatgaactgggtcagacaggctcctggaaagggcctggaatgggtgtctgtgattgaatc |
| | | caaggggaactacatcttctacgcggacagcgtgaagggccggttcactatcagcagagacaacagcaagaacaccc |
| | | tgtacctccaaatgaactcgctgagggccgaagatactgccgtgtactactgtgcccgcgatcgctactcgatgatctac |
| | | agctatggtgccggagcgttcgattactggggacagggaaccctcgtgaccgtcagctcc |
| SEQ ID NO: 15 | Heavy Chain | EVQLLESGGGLVQPGGSLRLSCAASGFTFSSYWMNWVRQAPGKGLEWVS |
| | | VIESKGNYIFYADSVKGRFTISRDNSKNTLYLQMNSLRAEDTAVYYCARDR |
| | | YSMIYSYGAGAFDYWGQGTLVTVSSASTKGPSVFPLAPSSKSTSGGTAALG |
| | | CLVKDYFPEPVTVSWNSGALTSGVHTFPAVLQSSGLYSLSSVVTVPSSSLGT |
| | | QTYICNVNHKPSNTKVDKRVEPKSCDKTHTCPPCPAPELLGGPSVFLFPPKP |
| | | KDTLMISRTPEVTCVVVAVSHEDPEVKFNWYVDGVEVHNAKTKPREEQY |
| | | NSTYRVVSVLTVLHQDWLNGKEYKCKVSNKALAAPIEKTISKAKGQPREP |
| | | QVYTLPPSREEMTKNQVSLTCLVKGFYPSDIAVEWESNGQPENNYKTTPPV |
| | | LDSDGSFFLYSKLTVDKSRWQQGNVFSCSVMHEALHNHYTQKSLSLSPGK |
| SEQ ID NO: 16 | DNA Heavy Chain | gaagtgcagctgctggaatccggcggaggtctggtccagcctggaggttccctgcgcctgtcatgcgcagcctccgg |
| | | attcaccttttcgtcgtactggatgaactgggtcagacaggctcctggaaagggcctggaatgggtgtctgtgattgaatc |
| | | caaggggaactacatcttctacgcggacagcgtgaagggccggttcactatcagcagagacaacagcaagaacaccc |
| | | tgtacctccaaatgaactcgctgagggccgaagatactgccgtgtactactgtgcccgcgatcgctactcgatgatctac |
| | | agctatggtgccggagcgttcgattactggggacagggaaccctcgtgaccgtcagctccgcctcaaccaagggccc |
| | | gtcagtgttcccgctggctccatcgtcgaagtccacctccggaggaaccgcagcactcggttgcctggtcaaggactac |
| | | ttccctgagccagtgaccgtgtcgtggaacagcggagccctgacttccggcgtgcacacttttcccgcggtgctgcagt |
| | | cctccggtctgtactccctttcgtccgtggtcaccgtgccgtcgtctagcctgggcacccagacctacatctgcaacgtg |
| | | aaccacaagccgtccaacaccaaagtggataagcgggtggagccgaagtcctgcgataagacacacacgtgcccgc |
| | | catgtccagcgcctgaattgcttggcggaccttccgtgttcctgttcccgcctaagcccaaggacaccttgatgattagcc |
| | | ggactcccgaagtcacctgtgtggtggtggcagtgtcccacgaggaccccgaggtcaagtttaattggtacgtggacg |
| | | gcgtcgaagtgcacaacgccaagactaagccccgggaggaacagtacaacagcacctaccgggtcgtgtccgtgct |
| | | gaccgtgctgcaccaggactggctgaatgggaaagagtacaagtgcaaagtgtccaacaaggccttggccgctcctat |
| | | cgaaaaaactatcagcaaggctaagggacagccgagggaaccccaagtctacaccctgcccccttcacgcgaagag |
| | | atgaccaagaatcaagtgtcgctgacctgcctcgtcaagggattctacccctccgacattgcggtggagtgggagtcca |
| | | acggccagcccgagaacaactacaagactactccgcccgtgctggactccgacggcagcttcttcctgtattccaagct |
| | | gaccgtggacaagtcccggtggcagcaaggaaacgtgttctcctgctcggtcatgcacgaagccctgcacaaccacta |
| | | tacgcagaagtccctgtccttgagcccggggaaa |
| SEQ ID NO: 17 | LCDR1 (Combined) | RASQGISSYLA |
| SEQ ID NO: 18 | LCDR2 (Combined) | TASTLQS |
| SEQ ID NO: 19 | LCDR3 (Combined) | QQTWRKPRT |
| SEQ ID NO: 17 | LCDR1 (Kabat) | RASQGISSYLA |
| SEQ ID NO: 18 | LCDR2 (Kabat) | TASTLQS |
| SEQ ID NO: 19 | LCDR3 (Kabat) | QQTWRKPRT |
| SEQ ID NO: 20 | LCDR1 (Chothia) | SQGISSY |
| SEQ ID NO: 21 | LCDR2 (Chothia) | TAS |
| SEQ ID NO: 22 | LCDR3 (Chothia) | TWRKPR |
| SEQ ID NO: 23 | LCDRI (IMGT) | QGISSY |
| SEQ ID NO: 21 | LCDR2 (IMGT) | TAS |
| SEQ ID NO: 19 | LCDR3 (IMGT) | QQTWRKPRT |
| SEQ ID NO: 24 | VL | DIQMTQSPSSLSASVGDRVTITCRASQGISSYLAWYQQKPGKAPKLLIYTAS |
| | | TLQSGVPSRFSGSGSGTDFTLTISSLQPEDFATYYCQQTWRKPRTFGQGTKV |
| | | EIK |
| SEQ ID NO: 25 | DNA VL | gacatccagatgacccagtctccgtcctccctgtccgcatcagtgggggacagagtgaccatcacttgtcgggcctccc |
| | | aaggcatctcgtcatacctggcctggtatcagcagaaacccggaaaggctccaaagctgctcatctacaccgcctcga |
| | | ctctgcaatccggagtgccttcccgcttctccggatccggttcgggaaccgacttcaccctcaccattagcagccttcag |
| | | ccggaagatttcgcgacctactactgccagcaaacctggcggaagcccaggacatttggccagggcactaaggtcga |
| | | gattaag |
| SEQ ID NO: 26 | Light Chain | DIQMTQSPSSLSASVGDRVTITCRASQGISSYLAWYQQKPGKAPKLLIYTAS |
| | | TLQSGVPSRFSGSGSGTDFTLTISSLQPEDFATYYCQQTWRKPRTFGQGTKV |
| | | EIKRTVAAPSVFIFPPSDEQLKSGTASVVCLLNNFYPREAKVQWKVDNALQ |
| | | SGNSQESVTEQDSKDSTYSLSSTLTLSKADYEKHKVYACEVTHQGLSSPVT |
| | | KSFNRGEC |
| SEQ ID NO: 27 | DNA Light Chain | gacatccagatgacccagtctccgtcctccctgtccgcatcagtgggggacagagtgaccatcacttgtcgggcctccc |
| | | aaggcatctcgtcatacctggcctggtatcagcagaaacccggaaaggctccaaagctgctcatctacaccgcctcga |
| | | ctctgcaatccggagtgccttcccgcttctccggatccggttcgggaaccgacttcaccctcaccattagcagccttcag |
| | | ccggaagatttcgcgacctactactgccagcaaacctggcggaagcccaggacatttggccagggcactaaggtcga |
| | | gattaagcgtacggtggccgctcccagcgtgttcatcttcccccccagcgacgagcagctgaagagcggcaccgcca |
| | | gcgtggtgtgcctgctgaacaacttctacccccgggaggccaaggtgcagtggaaggtggacaacgccctgcagag |
| | | cggcaacagccaggagagcgtcaccgagcaggacagcaaggactccacctacagcctgagcagcaccctgaccct |
| | | gagcaaggccgactacgagaagcataaggtgtacgcctgcgaggtgacccaccagggcctgtccagccccgtgacc |
| | | aagagcttcaacaggggcgagtgc |

N001_LALA

| | | |
|---|---|---|
| SEQ ID NO: 4 | HCDR1 (Combined) | GFTFSSYWMN |
| SEQ ID NO: 5 | HCDR2 (Combined) | VIESKGNYIFYADSVKG |
| SEQ ID NO: 6 | HCDR3 (Combined) | DRYSMIYSYGAGAFDY |
| SEQ ID NO: 7 | HCDR1 (Kabat) | SYWMN |
| SEQ ID NO: 5 | HCDR2 (Kabat) | VIESKGNYIFYADSVKG |
| SEQ ID NO: 6 | HCDR3 (Kabat) | DRYSMIYSYGAGAFDY |
| SEQ ID NO: 8 | HCDR1 (Chothia) | GFTFSSY |
| SEQ ID NO: 9 | HCDR2 (Chothia) | ESKGNY |
| SEQ ID NO: 6 | HCDR3 (Chothia) | DRYSMIYSYGAGAFDY |
| SEQ ID NO: 10 | HCDR1 (IMGT) | GFTFSSYW |
| SEQ ID NO: 11 | HCDR2 (IMGT) | IESKGNYI |
| SEQ ID NO: 12 | HCDR3 (IMGT) | ARDRYSMIYSYGAGAFDY |
| SEQ ID NO: 28 | VH | QVQLLESGGGLVQPGGSLRLSCAASGFTFSSYWMNWVRQAPGKGLEWVS |
| | | VIESKGNYIFYADSVKGRFTISRDNSKNTLYLQMNSLRAEDTAVYYCARDR |
| | | YSMIYSYGAGAFDYWGQGTLVTVSS |
| SEQ ID NO: 29 | DNA VH | caagttcaattgctggaaagcggaggtggacttgtccaacctggagggtcactccgactgtcttgcgctgcatcaggatt |
| | | cacctttagtagctattggatgaactgggtccggcaggctcctgggaaagggcttgagtgggtaagtgtcattgaatcaa |
| | | agggcaactacatcttttatgctgattctgtaaagggtaggttcaccatctccagggacaattcaaaaaatactttgtatctg |
| | | cagatgaactctctcagggcagaagacacggccgtttattactgcgcccgcgatcgatacagcatgatatactcctacg |
| | | gcgcaggagcttttgactactggggtcaaggcacacttgttactgtcagtagc |
| SEQ ID NO: 30 | Heavy Chain | QVQLLESGGGLVQPGGSLRLSCAASGFTFSSYWMNWVRQAPGKGLEWVS |
| | | VIESKGNYIFYADSVKGRFTISRDNSKNTLYLQMNSLRAEDTAVYYCARDR |
| | | YSMIYSYGAGAFDYWGQGTLVTVSSASTKGPSVFPLAPSSKSTSGGTAALG |
| | | CLVKDYFPEPVTVSWNSGALTSGVHTFPAVLQSSGLYSLSSVVTVPSSSLGT |
| | | QTYICNVNHKPSNTKVDKRVEPKSCDKTHTCPPCPAPEAAGGPSVFLFPPKP |
| | | KDTLMISRTPEVTCVVVDVSHEDPEVKFNWYVDGVEVHNAKTKPREEQY |
| | | NSTYRVVSVLTVLHQDWLNGKEYKCKVSNKALPAPIEKTISKAKGQPREP |
| | | QVYTLPPSREEMTKNQVSLTCLVKGFYPSDIAVEWESNGQPENNYKTTPPV |
| | | LDSDGSFFLYSKLTVDKSRWQQGNVFSCSVMHEALHNHYTQKSLSLSPGK |
| SEQ ID NO: 31 | DNA Heavy Chain | caagttcaattgctggaaagcggaggtggacttgtccaacctggagggtcactccgactgtcttgcgctgcatcaggatt |
| | | cacctttagtagctattggatgaactgggtccggcaggctcctgggaaagggcttgagtgggtaagtgtcattgaatcaa |
| | | agggcaactacatcttttatgctgattctgtaaagggtaggttcaccatctccagggacaattcaaaaaatactttgtatctg |
| | | cagatgaactctctcagggcagaagacacggccgtttattactgcgcccgcgatcgatacagcatgatatactcctacg |
| | | gcgcaggagcttttgactactggggtcaaggcacacttgttactgtcagtagcgcctcaacgaaaggaccgtccgtgttt |
| | | cctcttgctcctagctccaaatccacctcaggtggaacggccgccctggggtgcctggtaaaggactatttcccagagc |
| | | cagttactgtgtcttggaattctggtgcattgacaagtggcgtacacacttttcccgcggtcctccaatctagtggtctgtac |
| | | tcactgtcctccgttgtgactgtcccaagtagctcacttggcacacagacttacatctgtaatgttaatcataagccgtcaaa |
| | | cacgaaggtggataagagggtagaacctaagtcatgtgacaaaacgcatacttgccccccctgccctgcgccggaag |
| | | ccgctggcggaccctccgtattcttgttccctccaaagccaaaggacactctgatgattagccggacaccggaggtcac |
| | | ttgtgttgtagttgacgtcagccatgaggatcctgaggtgaaatttaattggtacgtggacggggttgaagtccacaatgc |
| | | taaaactaaacctagggaagagcaatataatagtacatacagggttgtcagtgtgctgaccgttctccatcaggactggc |
| | | tgaacggcaaggaatacaagtgcaaggtcagcaacaaggccttgccggcccccatcgagaagacgatctccaaagc |
| | | caaggggcaaccccgagaaccgcaggtatacacgctcccccctagtagagaagagatgacaaagaatcaagtttcctt |
| | | gacgtgccttgtgaaaggcttctaccctagtgacatcgcagtcgaatgggagagcaacgggcagccggagaataacta |
| | | taaaacaaccccccccgtgcttgactcagacgggtcattttttctgtatagcaaattgactgttgataaatcacggtggcaa |
| | | caaggaaacgtgtttagttgcagcgtaatgcacgaagctctccacaatcactatactcaaaagtcactgtcactctcccct |
| | | ggcaag |
| SEQ ID NO: 17 | LCDR1 (Combined) | RASQGISSYLA |
| SEQ ID NO: 18 | LCDR2 (Combined) | TASTLQS |
| SEQ ID NO: 19 | LCDR3 (Combined) | QQTWRKPRT |
| SEQ ID NO: 17 | LCDR1 (Kabat) | RASQGISSYLA |
| SEQ ID NO: 18 | LCDR2 (Kabat) | TASTLQS |
| SEQ ID NO: 19 | LCDR3 (Kabat) | QQTWRKPRT |
| SEQ ID NO: 20 | LCDR1 (Chothia) | SQGISSY |
| SEQ ID NO: 21 | LCDR2 (Chothia) | TAS |
| SEQ ID NO: 22 | LCDR3 (Chothia) | TWRKPR |
| SEQ ID NO: 23 | LCDR1 (IMGT) | QGISSY |
| SEQ ID NO: 21 | LCDR2 (IMGT) | TAS |
| SEQ ID NO: 19 | LCDR3 (IMGT) | QQTWRKPRT |
| SEQ ID NO: 24 | VL | DIQMTQSPSSLSASVGDRVTITCRASQGISSYLAWYQQKPGKAPKLLIYTAS |
| | | TLQSGVPSRFSGSGSGTDFTLTISSLQPEDFATYYCQQTWRKPRTFGQGTKV |
| | | EIK |
| SEQ ID NO: 32 | DNA VL | gacattcaaatgacacaaagtccgtccagtcttagtgcttctgtgggcgatagggtcaccatcacttgtcgggcgtctcag |
| | | gggatcagctcttacttggcatggtatcaacaaaagccaggaaaagcacctaaattgcttatttatacagcgtccaccctc |
| | | cagtcaggagtgcctagtaggttctcaggctctgggtccggtactgacttcacgctgactatatcaagcttgcaacccga |
| | | agattttgcaacatactactgccaacagacatggaggaagccaagaactttcggtcagggaacgaaagttgagataaa |
| | | g |
| SEQ ID NO: 26 | Light Chain | DIQMTQSPSSLSASVGDRVTITCRASQGISSYLAWYQQKPGKAPKLLIYTAS |
| | | TLQSGVPSRFSGSGSGTDFTLTISSLQPEDFATYYCQQTWRKPRTFGQGTKV |
| | | EIKRTVAAPSVFIFPPSDEQLKSGTASVVCLLNNFYPREAKVQWKVDNALQ |
| | | SGNSQESVTEQDSKDSTYSLSSTLTLSKADYEKHKVYACEVTHQGLSSPVT |
| | | KSFNRGEC |
| SEQ ID NO: 33 | DNA Light Chain | gacattcaaatgacacaaagtccgtccagtcttagtgcttctgtgggcgatagggtcaccatcacttgtcgggcgtctcag |
| | | gggatcagctcttacttggcatggtatcaacaaaagccaggaaaagcacctaaattgcttatttatacagcgtccaccctc |
| | | cagtcaggagtgcctagtaggttctcaggctctgggtccggtactgacttcacgctgactatatcaagcttgcaacccga |
| | | agattttgcaacatactactgccaacagacatggaggaagccaagaactttcggtcagggaacgaaagttgagataaa |
| | | gcgcactgtcgcagcaccttccgtgttcattttcccgccttccgacgagcagcttaaatcagggaccgcgagtgttgtttg |
| | | cttgcttaataacttttacccacgggaagccaaagttcagtggaaggtggacaatgcactccaaagcgggaatagtcag |
| | | gagtcagttactgagcaagatagtaaagactctacttactctttgagttcaaccttgaccctctcaaaagcggactacgag |
| | | aagcataaagtgtacgcctgcgaggtgacgcatcaaggtttgtcttccccggttacgaagtcctttaataggggggaatg |
| | | t |

N002_DAPA

| | | |
|---|---|---|
| SEQ ID NO: 4 | HCDR1 (Combined) | GFTFSSYWMN |
| SEQ ID NO: 5 | HCDR2 (Combined) | VIESKGNYIFYADSVKG |
| SEQ ID NO: 6 | HCDR3 (Combined) | DRYSMIYSYGAGAFDY |
| SEQ ID NO: 7 | HCDR1 (Kabat) | SYWMN |
| SEQ ID NO: 5 | HCDR2 (Kabat) | VIESKGNYIFYADSVKG |
| SEQ ID NO: 6 | HCDR3 (Kabat) | DRYSMIYSYGAGAFDY |
| SEQ ID NO: 8 | HCDR1 (Chothia) | GFTFSSY |
| SEQ ID NO: 9 | HCDR2 (Chothia) | ESKGNY |
| SEQ ID NO: 6 | HCDR3 (Chothia) | DRYSMIYSYGAGAFDY |
| SEQ ID NO: 10 | HCDR1 (IMGT) | GFTFSSYW |
| SEQ ID NO: 11 | HCDR2 (IMGT) | IESKGNYI |
| SEQ ID NO: 12 | HCDR3 (IMGT) | ARDRYSMIYSYGAGAFDY |
| SEQ ID NO: 13 | VH | EVQLLESGGGLVQPGGSLRLSCAASGFTFSSYWMNWVRQAPGKGLEWVS |
| | | VIESKGNYIFYADSVKGRFTISRDNSKNTLYLQMNSLRAEDTAVYYCARDR |
| | | YSMIYSYGAGAFDYWGQGTLVTVSS |
| SEQ ID NO: 14 | DNA VH | gaagtgcagctgctggaatccggcggaggtctggtccagcctggaggttccctgcgcctgtcatgcgcagcctccgg |
| | | attcaccttttcgtcgtactggatgaactgggtcagacaggctcctggaaagggcctggaatgggtgtctgtgattgaatc |
| | | caaggggaactacatcttctacgcggacagcgtgaagggccggttcactatcagcagagacaacagcaagaacaccc |
| | | tgtacctccaaatgaactcgctgagggccgaagatactgccgtgtactactgtgcccgcgatcgctactcgatgatctac |
| | | agctatggtgccggagcgttcgattactggggacagggaaccctcgtgaccgtcagctcc |
| SEQ ID NO: 15 | Heavy Chain | EVQLLESGGGLVQPGGSLRLSCAASGFTFSSYWMNWVRQAPGKGLEWVS |
| | | VIESKGNYIFYADSVKGRFTISRDNSKNTLYLQMNSLRAEDTAVYYCARDR |
| | | YSMIYSYGAGAFDYWGQGTLVTVSSASTKGPSVFPLAPSSKSTSGGTAALG |
| | | CLVKDYFPEPVTVSWNSGALTSGVHTFPAVLQSSGLYSLSSVVTVPSSSLGT |
| | | QTYICNVNHKPSNTKVDKRVEPKSCDKTHTCPPCPAPELLGGPSVFLFPPKP |
| | | KDTLMISRTPEVTCVVVAVSHEDPEVKFNWYVDGVEVHNAKTKPREEQY |
| | | NSTYRVVSVLTVLHQDWLNGKEYKCKVSNKALAAPIEKTISKAKGQPREP |
| | | QVYTLPPSREEMTKNQVSLTCLVKGFYPSDIAVEWESNGQPENNYKTTPPV |
| | | LDSDGSFFLYSKLTVDKSRWQQGNVFSCSVMHEALHNHYTQKSLSLSPGK |
| SEQ ID NO: 16 | DNA Heavy Chain | gaagtgcagctgctggaatccggcggaggtctggtccagcctggaggttccctgcgcctgtcatgcgcagcctccgg |
| | | attcaccttttcgtcgtactggatgaactgggtcagacaggctcctggaaagggcctggaatgggtgtctgtgattgaatc |
| | | caaggggaactacatcttctacgcggacagcgtgaagggccggttcactatcagcagagacaacagcaagaacaccc |
| | | tgtacctccaaatgaactcgctgagggccgaagatactgccgtgtactactgtgcccgcgatcgctactcgatgatctac |
| | | agctatggtgccggagcgttcgattactggggacagggaaccctcgtgaccgtcagctccgcctcaaccaagggccc |
| | | gtcagtgttcccgctggctccatcgtcgaagtccacctccggaggaaccgcagcactcggttgcctggtcaaggactac |
| | | ttccctgagccagtgaccgtgtcgtggaacagcggagccctgacttccggcgtgcacacttttcccgcggtgctgcagt |
| | | cctccggtctgtactccctttcgtccgtggtcaccgtgccgtcgtctagcctgggcacccagacctacatctgcaacgtg |
| | | aaccacaagccgtccaacaccaaagtggataaggggtggagccgaagtcctgcgataagacacacacgtgcccgc |
| | | catgtccagcgcctgaattgcttggcggaccttccgtgttcctgttcccgcctaagcccaaggacaccttgatgattagcc |
| | | ggactcccgaagtcacctgtgtggtggtggcagtgtcccacgaggaccccgaggtcaagtttaattggtacgtggacg |
| | | gcgtcgaagtgcacaacgccaagactaagccccgggaggaacagtacaacagcacctaccgggtcgtgtccgtgct |
| | | gaccgtgctgcaccaggactggctgaatgggaaagagtacaagtgcaaagtgtccaacaaggccttggccgctcctat |
| | | cgaaaaaactatcagcaaggctaagggacagccgagggaaccccaagtctacaccctgcccccttcacgcgaagag |
| | | atgaccaagaatcaagtgtcgctgacctgcctcgtcaagggattctacccctccgacattgcggtggagtgggagtcca |
| | | acggccagcccgagaacaactacaagactactccgcccgtgctggactccgacggcagcttcttcctgtattccaagct |
| | | gaccgtggacaagtcccggtggcagcaaggaaacgtgttctcctgctcggtcatgcacgaagccctgcacaaccacta |
| | | tacgcagaagtccctgtccttgagcccggggaaa |
| SEQ ID NO: 17 | LCDR1 (Combined) | RASQGISSYLA |
| SEQ ID NO: 18 | LCDR2 (Combined) | TASTLQS |
| SEQ ID NO: 34 | LCDR3 (Combined) | QQEWVKPRT |
| SEQ ID NO: 17 | LCDR1 (Kabat) | RASQGISSYLA |
| SEQ ID NO: 18 | LCDR2 (Kabat) | TASTLQS |
| SEQ ID NO: 34 | LCDR3 (Kabat) | QQEWVKPRT |
| SEQ ID NO: 20 | LCDRI (Chothia) | SQGISSY |
| SEQ ID NO: 21 | LCDR2 (Chothia) | TAS |
| SEQ ID NO: 35 | LCDR3 (Chothia) | EWVKPR |
| SEQ ID NO: 23 | LCDR1 (IMGT) | QGISSY |
| SEQ ID NO: 21 | LCDR2 (IMGT) | TAS |
| SEQ ID NO: 34 | LCDR3 (IMGT) | QQEWVKPRT |
| SEQ ID NO: 36 | VL | DIQMTQSPSSLSASVGDRVTITCRASQGISSYLAWYQQKPGKAPKLLIYTAS |
| | | TLQSGVPSRFSGSGSGTDFTLTISSLQPEDFATYYCQQEWVKPRTFGQGTKV |
| | | EIK |
| SEQ ID NO: 37 | DNA VL | gacatccagatgacccagtctccgtcctccctgtccgcatcagtgggggacagagtgaccatcacttgtcgggcctccc |
| | | aaggcatctcgtcatacctggcctggtatcagcagaaacccggaaaggctccaaagctgctcatctacaccgcctoga |
| | | ctctgcaatccggagtgccttcccgcttctccggatccggttcgggaaccgacttcaccctcaccattagcagccttcag |
| | | ccggaagatttcgcgacctactactgccagcaagaatgggtgaagcccaggacatttggccagggcactaaggtcga |
| | | gattaag |
| SEQ ID NO: 38 | Light Chain | DIQMTQSPSSLSASVGDRVTITCRASQGISSYLAWYQQKPGKAPKLLIYTAS |
| | | TLQSGVPSRFSGSGSGTDFTLTISSLQPEDFATYYCQQEWVKPRTFGQGTKV |
| | | EIKRTVAAPSVFIFPPSDEQLKSGTASVVCLLNNFYPREAKVQWKVDNALQ |
| | | SGNSQESVTEQDSKDSTYSLSSTLTLSKADYEKHKVYACEVTHQGLSSPVT |
| | | KSFNRGEC |
| SEQ ID NO: 39 | DNA Light Chain | gacatccagatgacccagtctccgtcctccctgtccgcatcagtgggggacagagtgaccatcacttgtcgggcctccc |
| | | aaggcatctcgtcatacctggcctggtatcagcagaaacccggaaaggctccaaagctgctcatctacaccgcctoga |
| | | ctctgcaatccggagtgccttcccgcttctccggatccggttcgggaaccgacttcaccctcaccattagcagccttcag |
| | | ccggaagatttcgcgacctactactgccagcaagaatgggtgaagcccaggacatttggccagggcactaaggtcga |
| | | gattaagcgtacggtggccgctcccagcgtgttcatcttcccccccagcgacgagcagctgaagagcggcaccgcca |
| | | gcgtggtgtgcctgctgaacaacttctacccccgggaggccaaggtgcagtggaaggtggacaacgccctgcagag |
| | | cggcaacagccaggagagcgtcaccgagcaggacagcaaggactccacctacagcctgagcagcaccctgaccct |
| | | gagcaaggccgactacgagaagcataaggtgtacgcctgcgaggtgacccaccagggcctgtccagccccgtgacc |
| | | aagagcttcaacaggggcgagtgc |

N002_LALA

| | | |
|---|---|---|
| SEQ ID NO: 4 | HCDR1 (Combined) | GFTFSSYWMN |
| SEQ ID NO: 5 | HCDR2 (Combined) | VIESKGNYIFYADSVKG |
| SEQ ID NO: 6 | HCDR3 (Combined) | DRYSMIYSYGAGAFDY |
| SEQ ID NO: 7 | HCDR1 (Kabat) | SYWMN |
| SEQ ID NO: 5 | HCDR2 (Kabat) | VIESKGNYIFYADSVKG |
| SEQ ID NO: 6 | HCDR3 (Kabat) | DRYSMIYSYGAGAFDY |
| SEQ ID NO: 8 | HCDR1 (Chothia) | GFTFSSY |
| SEQ ID NO: 9 | HCDR2 (Chothia) | ESKGNY |
| SEQ ID NO: 6 | HCDR3 (Chothia) | DRYSMIYSYGAGAFDY |
| SEQ ID NO: 10 | HCDR1 (IMGT) | GFTFSSYW |
| SEQ ID NO: 11 | HCDR2 (IMGT) | IESKGNYI |
| SEQ ID NO: 12 | HCDR3 (IMGT) | ARDRYSMIYSYGAGAFDY |
| SEQ ID NO: 28 | VH | QVQLLESGGGLVQPGGSLRLSCAASGFTFSSYWMNWVRQAPGKGLEWVS |
| | | VIESKGNYIFYADSVKGRFTISRDNSKNTLYLQMNSLRAEDTAVYYCARDR |
| | | YSMIYSYGAGAFDYWGQGTLVTVSS |
| SEQ ID NO: 29 | DNA VH | caagttcaattgctggaaagcggaggtggacttgtccaacctggagggtcactccgactgtcttgcgctgcatcaggatt |
| | | cacctttagtagctattggatgaactgggtccggcaggctcctgggaaagggcttgagtgggtaagtgtcattgaatcaa |
| | | agggcaactacatcttttatgctgattctgtaaagggtaggttcaccatctccagggacaattcaaaaaatactttgtatctg |
| | | cagatgaactctctcagggcagaagacacggccgtttattactgcgcccgcgatcgatacagcatgatatactcctacg |
| | | gcgcaggagcttttgactactggggtcaaggcacacttgttactgtcagtagc |
| SEQ ID NO: 30 | Heavy Chain | QVQLLESGGGLVQPGGSLRLSCAASGFTFSSYWMNWVRQAPGKGLEWVS |
| | | VIESKGNYIFYADSVKGRFTISRDNSKNTLYLQMNSLRAEDTAVYYCARDR |
| | | YSMIYSYGAGAFDYWGQGTLVTVSSASTKGPSVFPLAPSSKSTSGGTAALG |
| | | CLVKDYFPEPVTVSWNSGALTSGVHTFPAVLQSSGLYSLSSVVTVPSSSLGT |
| | | QTYICNVNHKPSNTKVDKRVEPKSCDKTHTCPPCPAPEAAGGPSVFLFPPKP |
| | | KDTLMISRTPEVTCVVVDVSHEDPEVKFNWYVDGVEVHNAKTKPREEQY |
| | | NSTYRVVSVLTVLHQDWLNGKEYKCKVSNKALPAPIEKTISKAKGQPREP |
| | | QVYTLPPSREEMTKNQVSLTCLVKGFYPSDIAVEWESNGQPENNYKTTPPV |
| | | LDSDGSFFLYSKLTVDKSRWQQGNVFSCSVMHEALHNHYTQKSLSLSPGK |
| SEQ ID NO: 31 | DNA Heavy Chain | caagttcaattgctggaaagcggaggtggacttgtccaacctggagggtcactccgactgtcttgcgctgcatcaggatt |
| | | cacctttagtagctattggatgaactgggtccggcaggctcctgggaaagggcttgagtgggtaagtgtcattgaatcaa |
| | | agggcaactacatcttttatgctgattctgtaaagggtaggttcaccatctccagggacaattcaaaaaatactttgtatctg |
| | | cagatgaactctctcagggcagaagacacggccgtttattactgcgcccgcgatcgatacagcatgatatactcctacg |
| | | gcgcaggagcttttgactactggggtcaaggcacacttgttactgtcagtagcgcctcaacgaaaggaccgtccgtgttt |
| | | cctcttgctcctagctccaaatccacctcaggtggaacggccgccctggggtgcctggtaaaggactatttcccagagc |
| | | cagttactgtgtcttggaattctggtgcattgacaagtggcgtacacacttttcccgcggtcctccaatctagtggtctgtac |
| | | tcactgtcctccgttgtgactgtcccaagtagctcacttggcacacagacttacatctgtaatgttaatcataagccgtcaaa |
| | | cacgaaggtggataagagggtagaacctaagtcatgtgacaaaacgcatacttgccccccctgccctgcgccggaag |
| | | ccgctggcggaccctccgtattcttgttccctccaaagccaaaggacactctgatgattagccggacaccggaggtcac |
| | | ttgtgttgtagttgacgtcagccatgaggatcctgaggtgaaatttaattggtacgtggacggggttgaagtccacaatgc |
| | | taaaactaaacctagggaagagcaatataatagtacatacagggttgtcagtgtgctgaccgttctccatcaggactggc |
| | | tgaacggcaaggaatacaagtgcaaggtcagcaacaaggccttgccggcccccatcgagaagacgatctccaaagc |
| | | caaggggcaaccccgagaaccgcaggtatacacgctcccccctagtagagaagagatgacaaagaatcaagtttcctt |
| | | gacgtgccttgtgaaaggcttctaccctagtgacatcgcagtcgaatgggagagcaacgggcagccggagaataacta |
| | | taaaacaaccccccccgtgcttgactcagacgggtcattttttctgtatagcaaattgactgttgataaatcacggtggcaa |
| | | caaggaaacgtgtttagttgcagcgtaatgcacgaagctctccacaatcactatactcaaaagtcactgtcactctcccct |
| | | ggcaag |
| SEQ ID NO: 17 | LCDR1 (Combined) | RASQGISSYLA |
| SEQ ID NO: 18 | LCDR2 (Combined) | TASTLQS |
| SEQ ID NO: 34 | LCDR3 (Combined) | QQEWVKPRT |
| SEQ ID NO: 17 | LCDR1 (Kabat) | RASQGISSYLA |
| SEQ ID NO: 18 | LCDR2 (Kabat) | TASTLQS |
| SEQ ID NO: 34 | LCDR3 (Kabat) | QQEWVKPRT |
| SEQ ID NO: 20 | LCDR1 (Chothia) | SQGISSY |
| SEQ ID NO: 21 | LCDR2 (Chothia) | TAS |
| SEQ ID NO: 35 | LCDR3 (Chothia) | EWVKPR |
| SEQ ID NO: 23 | LCDR1 (IMGT) | QGISSY |
| SEQ ID NO: 21 | LCDR2 (IMGT) | TAS |
| SEQ ID NO: 34 | LCDR3 (IMGT) | QQEWVKPRT |
| SEQ ID NO: 36 | VL | DIQMTQSPSSLSASVGDRVTITCRASQGISSYLAWYQQKPGKAPKLLIYTAS |
| | | TLQSGVPSRFSGSGSGTDFTLTISSLQPEDFATYYCQQEWVKPRTFGQGTKV |
| | | EIK |
| SEQ ID NO: 40 | DNA VL | gacatacaaatgacgcaatctccgagtagcttgtcagcgtccgtaggcgaccgagtaacgattacgtgtagagcgagc |
| | | cagggaatttcatcttatttggcttggtatcagcaaaagccgggaaaagcacccaaactcctcatttatactgccagcacg |
| | | ttgcaaagcggcgttccgagtcggttctctggatcagggtccgggacggacttcaccttgacgatttcatctttgcaacct |
| | | gaagattttgcaacatactactgtcaacaggagtgggtgaagccaaggaccttcggacaaggcacgaaggtcgaaatc |
| | | aag |
| SEQ ID NO: 38 | Light Chain | DIQMTQSPSSLSASVGDRVTITCRASQGISSYLAWYQQKPGKAPKLLIYTAS |
| | | TLQSGVPSRFSGSGSGTDFTLTISSLQPEDFATYYCQQEWVKPRTFGQGTKV |
| | | EIKRTVAAPSVFIFPPSDEQLKSGTASVVCLLNNFYPREAKVQWKVDNALQ |
| | | SGNSQESVTEQDSKDSTYSLSSTLTLSKADYEKHKVYACEVTHQGLSSPVT |
| | | KSFNRGEC |
| SEQ ID NO: 41 | DNA Light Chain | gacatacaaatgacgcaatctccgagtagcttgtcagcgtccgtaggcgaccgagtaacgattacgtgtagagcgagc |
| | | cagggaatttcatcttatttggcttggtatcagcaaaagccgggaaaagcacccaaactcctcatttatactgccagcacg |
| | | ttgcaaagcggcgttccgagtcggttctctggatcagggtccgggacggacttcaccttgacgatttcatctttgcaacct |
| | | gaagattttgcaacatactactgtcaacaggagtgggtgaagccaaggaccttcggacaaggcacgaaggtcgaaatc |
| | | aagcgaaccgtggcagctccgtccgtgtttatttttccgccttccgacgaacaacttaaaagtggaacagcctctgtcgtc |
| | | tgtctccttaacaacttctaccccagggaagctaaagtacagtggaaggtagataacgctctgcaaagtggtaattctcag |
| | | gagagcgtcacggaacaggactccaaagactccacctattctctgagctctacactgacgctcagcaaggcagactac |
| | | gaaaagcacaaagtatatgcgtgtgaggtgacgcatcaaggccttagcagtccagttacaaaaagttttaacaggggag |
| | | aatgc |

For example, N001_DAPA may be defined as having three heavy chain complementarity determining regions (HCDR1, HCDR2, and HCDR3) and three light chain complementarity determining regions (LCDR1, LCDR2, and LCDR3) selected from: (I) SEQ ID NO: 4 (HCDR1), SEQ ID NO: 5 (HCDR2), SEQ ID NO: 6 (HCDR3), SEQ ID NO: 17 (LCDR1), SEQ ID NO: 18 (LCDR2), and SEQ ID NO: 19 (LCDR3); (II) SEQ ID NO: 7 (HCDR1), SEQ ID NO: 5 (HCDR2), SEQ ID NO: 6 (HCDR3), SEQ ID NO: 17 (LCDR1), SEQ ID NO: 18 (LCDR2), and SEQ ID NO: 19 (LCDR3); (III) SEQ ID NO: 8 (HCDR1), SEQ ID NO: 9 (HCDR2), SEQ ID NO: 6 (HCDR3), SEQ ID NO: 20 (LCDR1), SEQ ID NO: 21 (LCDR2), and SEQ ID NO: 22 (LCDR3); or (IV) SEQ ID NO: 10 (HCDR1), SEQ ID NO: 11 (HCDR2), SEQ ID NO: 12 (HCDR3), SEQ ID NO: 23 (LCDR1), SEQ ID NO: 21 (LCDR2), and SEQ ID NO: 19 (LCDR3). In some embodiments, N001_DAPA may be defined as comprising or having amino acid sequences of SEQ ID NO: 4 (HCDR1), SEQ ID NO: 5 (HCDR2), SEQ ID NO: 6 (HCDR3), SEQ ID NO: 17 (LCDR1), SEQ ID NO: 18 (LCDR2), and SEQ ID NO: 19 (LCDR3). In some embodiments, N001_DAPA may be defined as comprising or having amino acid sequences of SEQ ID NO: 7 (HCDR1), SEQ ID NO: 5 (HCDR2), SEQ ID NO: 6 (HCDR3), SEQ ID NO: 17 (LCDR1), SEQ ID NO: 18 (LCDR2), and SEQ ID NO: 19 (LCDR3). In some embodiments, N001_DAPA may be defined as comprising or having amino acid sequences of SEQ ID NO: 8 (HCDR1), SEQ ID NO: 9 (HCDR2), SEQ ID NO: 6 (HCDR3), SEQ ID NO: 20 (LCDR1), SEQ ID NO: 21 (LCDR2), and SEQ ID NO: 22 (LCDR3). In some embodiments, N001_DAPA may be defined as comprising or having amino acid sequences of SEQ ID NO: 10 (HCDR1), SEQ ID NO: 11 (HCDR2), SEQ ID NO: 12 (HCDR3), SEQ ID NO: 23 (LCDR1), SEQ ID NO: 21 (LCDR2), and SEQ ID NO: 19 (LCDR3). In some embodiments, N001_DAPA may be defined as comprising or having a heavy chain variable region comprising or having an amino acid sequence of SEQ ID NO: 13, and a light chain variable region comprising or having an amino acid sequence of SEQ ID NO: 24. In some embodiments, N001_DAPA may be defined as comprising or having a heavy chain comprising or having an amino acid sequence of SEQ ID NO: 15, and a light chain comprising or having an amino acid sequence of SEQ ID NO: 26.

For example, N001_LALA may be defined as having three heavy chain complementarity determining regions (HCDR1, HCDR2, and HCDR3) and three light chain complementarity determining regions (LCDR1, LCDR2, and LCDR3) selected from: (I) SEQ ID NO: 4 (HCDR1), SEQ ID NO: 5 (HCDR2), SEQ ID NO: 6 (HCDR3), SEQ ID NO: 17 (LCDR1), SEQ ID NO: 18 (LCDR2), and SEQ ID NO: 19 (LCDR3); (II) SEQ ID NO: 7 (HCDR1), SEQ ID NO: 5 (HCDR2), SEQ ID NO: 6 (HCDR3), SEQ ID NO: 17 (LCDR1), SEQ ID NO: 18 (LCDR2), and SEQ ID NO: 19 (LCDR3); (III) SEQ ID NO: 8 (HCDR1), SEQ ID NO: 9 (HCDR2), SEQ ID NO: 6 (HCDR3), SEQ ID NO: 20 (LCDR1), SEQ ID NO: 21 (LCDR2), and SEQ ID NO: 22 (LCDR3); or (IV) SEQ ID NO: 10 (HCDR1), SEQ ID NO: 11 (HCDR2), SEQ ID NO: 12 (HCDR3), SEQ ID NO: 23 (LCDR1), SEQ ID NO: 21 (LCDR2), and SEQ ID NO: 19 (LCDR3). In some embodiments, N001_LALA may be defined as comprising or having amino acid sequences of SEQ ID NO: 4 (HCDR1), SEQ ID NO: 5 (HCDR2), SEQ ID NO: 6 (HCDR3), SEQ ID NO: 17 (LCDR1), SEQ ID NO: 18 (LCDR2), and SEQ ID NO: 19 (LCDR3). In some embodiments, N001_LALA may be defined as comprising or having amino acid sequences of SEQ ID NO: 7 (HCDR1), SEQ ID NO: 5 (HCDR2), SEQ ID NO: 6 (HCDR3), SEQ ID NO: 17 (LCDR1), SEQ ID NO: 18 (LCDR2), and SEQ ID NO: 19 (LCDR3). In some embodiments, N001_LALA may be defined as comprising or having amino acid sequences of SEQ ID NO: 8 (HCDR1), SEQ ID NO: 9 (HCDR2), SEQ ID NO: 6 (HCDR3), SEQ ID NO: 20 (LCDR1), SEQ ID NO: 21 (LCDR2), and SEQ ID NO: 22 (LCDR3). In some embodiments, N001_LALA may be defined as comprising or having amino acid sequences of SEQ ID NO: 10 (HCDR1), SEQ ID NO: 11 (HCDR2), SEQ ID NO: 12 (HCDR3), SEQ ID NO: 23 (LCDR1), SEQ ID NO: 21 (LCDR2), and SEQ ID NO: 19 (LCDR3). In some embodiments, N001_LALA may be defined as comprising or having a heavy chain variable region comprising or having an amino acid sequence of SEQ ID NO: 28, and a light chain variable region comprising or having an amino acid sequence of SEQ ID NO: 24. In some embodiments, N001_LALA may be defined as comprising or having a heavy chain comprising or having an amino acid sequence of SEQ ID NO: 30, and a light chain comprising or having an amino acid sequence of SEQ ID NO: 26.

For example, N002_DAPA may be defined as having three heavy chain complementarity determining regions (HCDR1, HCDR2, and HCDR3) and three light chain complementarity determining regions (LCDR1, LCDR2, and LCDR3) selected from: (I) SEQ ID NO: 4 (HCDR1), SEQ ID NO: 5 (HCDR2), SEQ ID NO: 6 (HCDR3), SEQ ID NO: 17 (LCDR1), SEQ ID NO: 18 (LCDR2), and SEQ ID NO: 34 (LCDR3); (II) SEQ ID NO: 7 (HCDR1), SEQ ID NO: 5 (HCDR2), SEQ ID NO: 6 (HCDR3), SEQ ID NO: 17 (LCDR1), SEQ ID NO: 18 (LCDR2), and SEQ ID NO: 34 (LCDR3); (III) SEQ ID NO: 8 (HCDR1), SEQ ID NO: 9 (HCDR2), SEQ ID NO: 6 (HCDR3), SEQ ID NO: 20 (LCDR1), SEQ ID NO: 21 (LCDR2), and SEQ ID NO: 35 (LCDR3); or (IV) SEQ ID NO: 10 (HCDR1), SEQ ID NO: 11 (HCDR2), SEQ ID NO: 12 (HCDR3), SEQ ID NO: 23 (LCDR1), SEQ ID NO: 21 (LCDR2), and SEQ ID NO: 34 (LCDR3). In some embodiments, N002_DAPA may be defined as comprising or having amino acid sequences of SEQ ID NO: 4 (HCDR1), SEQ ID NO: 5 (HCDR2), SEQ ID NO: 6 (HCDR3), SEQ ID NO: 17 (LCDR1), SEQ ID NO: 18 (LCDR2), and SEQ ID NO: 34 (LCDR3). In some embodiments, N002_DAPA may be defined as comprising or having amino acid sequences of SEQ ID NO: 7 (HCDR1), SEQ ID NO: 5 (HCDR2), SEQ ID NO: 6 (HCDR3), SEQ ID NO: 17 (LCDR1), SEQ ID NO: 18 (LCDR2), and SEQ ID NO: 34 (LCDR3). In some embodiments, N002_DAPA may be defined as comprising or having amino acid sequences of SEQ ID NO: 8 (HCDR1), SEQ ID NO: 9 (HCDR2), SEQ ID NO: 6 (HCDR3), SEQ ID NO: 20 (LCDR1), SEQ ID NO: 21 (LCDR2), and SEQ ID NO: 35 (LCDR3). In some embodiments, N002_DAPA may be defined as comprising or having amino acid sequences of SEQ ID NO: 10 (HCDR1), SEQ ID NO: 11 (HCDR2), SEQ ID NO: 12 (HCDR3), SEQ ID NO: 23 (LCDR1), SEQ ID NO: 21 (LCDR2), and SEQ ID NO: 34 (LCDR3). In some embodiments, N002_DAPA may be defined as comprising or having a heavy chain variable region comprising or having an amino acid sequence of SEQ ID NO: 13, and a light chain variable region comprising or having an amino acid sequence of SEQ ID NO: 36. In some embodiments, N002_DAPA may be defined as comprising or having a heavy chain comprising or having an amino acid sequence of SEQ ID NO: 15, and a light chain comprising or having an amino acid sequence of SEQ ID NO: 38.

For example, N002_LALA may be defined as having three heavy chain complementarity determining regions (HCDR1, HCDR2, and HCDR3) and three light chain complementarity determining regions (LCDR1, LCDR2, and LCDR3) selected from: (I) SEQ ID NO: 4 (HCDR1), SEQ ID NO: 5 (HCDR2), SEQ ID NO: 6 (HCDR3), SEQ ID NO: 17 (LCDR1), SEQ ID NO: 18 (LCDR2), and SEQ ID NO: 34 (LCDR3); (II) SEQ ID NO: 7 (HCDR1), SEQ ID NO: 5 (HCDR2), SEQ ID NO: 6 (HCDR3), SEQ ID NO: 17 (LCDR1), SEQ ID NO: 18 (LCDR2), and SEQ ID NO: 34 (LCDR3); (III) SEQ ID NO: 8 (HCDR1), SEQ ID NO: 9 (HCDR2), SEQ ID NO: 6 (HCDR3), SEQ ID NO: 20 (LCDR1), SEQ ID NO: 21 (LCDR2), and SEQ ID NO: 35 (LCDR3); or (IV) SEQ ID NO: 10 (HCDR1), SEQ ID NO: 11 (HCDR2), SEQ ID NO: 12 (HCDR3), SEQ ID NO: 23 (LCDR1), SEQ ID NO: 21 (LCDR2), and SEQ ID NO: 34 (LCDR3). In some embodiments, N002_LALA may be defined as comprising or having amino acid sequences of SEQ ID NO: 4 (HCDR1), SEQ ID NO: 5 (HCDR2), SEQ ID NO: 6 (HCDR3), SEQ ID NO: 17 (LCDR1), SEQ ID NO: 18 (LCDR2), and SEQ ID NO: 34 (LCDR3). In some embodiments, N002_LALA may be defined as comprising or having amino acid sequences of SEQ ID NO: 7 (HCDR1), SEQ ID NO: 5 (HCDR2), SEQ ID NO: 6 (HCDR3), SEQ ID NO: 17 (LCDR1), SEQ ID NO: 18 (LCDR2), and SEQ ID NO: 34 (LCDR3). In some embodiments, N002_LALA may be defined as comprising or having amino acid sequences of SEQ ID NO: 8 (HCDR1), SEQ ID NO: 9 (HCDR2), SEQ ID NO: 6 (HCDR3), SEQ ID NO: 20 (LCDR1), SEQ ID NO: 21 (LCDR2), and SEQ ID NO: 35 (LCDR3). In some embodiments, N002_LALA may be defined as comprising or having amino acid sequences of SEQ ID NO: 10 (HCDR1), SEQ ID NO: 11 (HCDR2), SEQ ID NO: 12 (HCDR3), SEQ ID NO: 23 (LCDR1), SEQ ID NO: 21 (LCDR2), and SEQ ID NO: 34 (LCDR3). In some embodiments, N002_LALA may be defined as comprising or having a heavy chain variable region comprising or having an amino acid sequence of SEQ ID NO: 28, and a light chain variable region comprising or having an amino acid sequence of SEQ ID NO: 36. In some embodiments, N002_LALA may be defined as comprising or having a heavy chain comprising or having an amino acid sequence of SEQ ID NO: 30, and a light chain comprising or having an amino acid sequence of SEQ ID NO: 38.

In some embodiments, the antibody or antigen binding fragment comprises a heavy chain variable region comprising or having an amino acid sequence of SEQ ID NO: 13 or a sequence having at least 95%, 96%, 97%, 98%, or 99% identity with SEQ ID NO: 13, and a light chain variable region comprising or having an amino acid sequence of SEQ ID NO: 24 or a sequence having at least 95%, 96%, 97%, 98%, or 99% identity with SEQ ID NO: 24. In some other embodiments, the antibody or antigen binding fragment comprises a heavy chain comprising or having an amino acid sequence of SEQ ID NO: 15 or a sequence having at least 95%, 96%, 97%, 98%, or 99% identity with SEQ ID NO: 15, and a light chain comprising or having an amino acid sequence of SEQ ID NO: 26 or a sequence having at least 95%, 96%, 97%, 98%, or 99% identity with SEQ ID NO: 26.

In some embodiments, the antibody or antigen binding fragment comprises a heavy chain variable region comprising or having an amino acid sequence of SEQ ID NO: 28 or a sequence having at least 95%, 96%, 97%, 98%, or 99% identity with SEQ ID NO: 28, and a light chain variable region comprising or having an amino acid sequence of SEQ ID NO: 24 or a sequence having at least 95%, 96%, 97%, 98%, or 99% identity with SEQ ID NO: 24. In some other embodiments, the antibody or antigen binding fragment comprises a heavy chain comprising or having an amino acid sequence of SEQ ID NO: 30 or a sequence having at least 95%, 96%, 97%, 98%, or 99% identity with SEQ ID NO: 30, and a light chain comprising or having an amino acid sequence of SEQ ID NO: 26 or a sequence having at least 95%, 96%, 97%, 98%, or 99% identity with SEQ ID NO: 26.

In some embodiments, the antibody or antigen binding fragment comprises a heavy chain variable region comprising or having an amino acid sequence of SEQ ID NO: 13 or a sequence having at least 95%, 96%, 97%, 98%, or 99% identity with SEQ ID NO: 13, and a light chain variable region comprising or having an amino acid sequence of SEQ ID NO: 36 or a sequence having at least 95%, 96%, 97%, 98%, or 99% identity with SEQ ID NO: 36. In some other embodiments, the antibody or antigen binding fragment comprises a heavy chain comprising or having an amino acid sequence of SEQ ID NO: 15 or a sequence having at least 95%, 96%, 97%, 98%, or 99% identity with SEQ ID NO: 15, and a light chain comprising or having an amino acid sequence of SEQ ID NO: 38 or a sequence having at least 95%, 96%, 97%, 98%, or 99% identity with SEQ ID NO: 38.

In some embodiments, the antibody or antigen binding fragment comprises a heavy chain variable region comprising or having an amino acid sequence of SEQ ID NO: 28 or a sequence having at least 95%, 96%, 97%, 98%, or 99% identity with SEQ ID NO: 28, and a light chain variable region comprising or having an amino acid sequence of SEQ ID NO: 36 or a sequence having at least 95%, 96%, 97%, 98%, or 99% identity with SEQ ID NO: 36. In some other embodiments, the antibody or antigen binding fragment comprises a heavy chain comprising or having an amino acid sequence of SEQ ID NO: 30 or a sequence having at least 95%, 96%, 97%, 98%, or 99% identity with SEQ ID NO: 30, and a light chain comprising or having an amino acid sequence of SEQ ID NO: 38 or a sequence having at least 95%, 96%, 97%, 98%, or 99% identity with SEQ ID NO: 38.

### Engineered and Modified Antibodies

An antibody described herein for any of the uses or methods described herein can be prepared using an antibody having one or more of the VH and/or VL sequences shown herein as starting material to engineer a modified antibody, which modified antibody may have altered properties from the starting antibody. An antibody can be engineered by modifying one or more residues within one or both variable regions (i.e., VH and/or VL), for example within one or more CDR regions and/or within one or more framework regions. Additionally or alternatively, an antibody can be engineered by modifying residues within the constant region(s), for example to alter the effector function(s) of the antibody.

One type of variable region engineering that can be performed is antibody binding region/paratope or CDR grafting. Because paratope sequences are responsible for most antibody-antigen interactions, it is possible to express recombinant antibodies that mimic the properties of specific naturally occurring antibodies by constructing expression vectors that include CDR/paratope sequences from the specific naturally occurring antibody grafted onto framework sequences from a different antibody with different properties (see, e.g., Riechmann, L. et al., 1998 Nature 332:323-327; Jones, P. et al., 1986 Nature 321:522-525; Queen, C. et al., 1989 Proc. Natl. Acad. See. U.S.A. 86:10029-10033; U.S. Pat. Nos. 5,225,539, and 5,530,101; 5,585,089; 5,693,762 and 6,180,370; the contents of each of which are herein incorporated by reference for this purpose).

Accordingly, another embodiment pertains to any of the uses or methods described herein in which the isolated anti-NPR1 antibody, or a antigen-binding fragment thereof, comprising an antigen binding portion thereof, comprising a heavy chain variable region comprising the CDR sequences of an antibody or group of antibodies shown in Table 2. Thus, such antibodies contain the VH and VL CDR sequences of monoclonal antibodies, yet may contain different framework sequences from these antibodies.

Such framework sequences can be obtained from public DNA databases or published references that include germline antibody gene sequences. For example, germline DNA sequences for human heavy and light chain variable region genes can be found in the “VBase” human germline sequence database (available on the Internet at www [dot] mrc-cpe [dot] cam [dot] ac [dot] uk/vbase), as well as in Kabat, E. A., et al., 1991 Sequences of Proteins of Immunological Interest, Fifth Edition, U.S. Department of Health and Human Services, NIH Publication No. 91-3242; Tomlinson, I. M., et al., 1992 J. Mol. Biol. 227:776-798; and Cox, J. P. L. et al., 1994 Eur. J Immunol. 24:827-836; the contents of each of which are herein incorporated by reference for this purpose.

An example of framework sequences for use in the antibodies or antigen binding fragments described herein are those that are structurally similar to the framework sequences used by selected antibodies described herein, e.g., consensus sequences and/or framework sequences used by the antibodies or antigen binding fragments described herein. The VH CDR1, CDR2, and CDR3 sequences and the VL CDR1, CDR2, and CDR3 sequences can be grafted onto framework regions that have an identical sequence to that found in the germline immunoglobulin gene from which the framework sequence derive, or the CDR sequences can be grafted onto framework regions that contain one or more mutations as compared to the germline sequences. For example, it has been found that in certain instances it is beneficial to mutate residues within the framework regions to maintain or enhance the antigen binding ability of the antibody (see e.g., U.S. Pat. Nos. 5,530,101; 5,585,089; 5,693,762 and 6,180,370; the contents of each of which are herein incorporated by reference for this purpose).

Another type of variable region modification is to mutate amino acid residues within the VH and/or VL CDR1, CDR2 and/or CDR3 regions to thereby improve one or more binding properties (e.g., affinity) of the antibody of interest, known as “affinity maturation.” Site-directed mutagenesis or PCR-mediated mutagenesis can be performed to introduce the mutation(s) and the effect on antibody binding, or other functional property of interest, can be evaluated in in vitro or in vivo assays as described herein. Conservative modifications (as discussed above) can also be introduced. The mutations may be amino acid substitutions, additions or deletions. Moreover, typically no more than one, two, three, four or five residues within a CDR region are altered.

Grafting Antigen Binding Domains into Alternative Frameworks or Scaffolds

A wide variety of antibody/immunoglobulin frameworks or scaffolds can be employed so long as the resulting polypeptide includes at least one binding region which specifically binds to NPR1. Such frameworks or scaffolds include the 5 main idiotypes of human immunoglobulins, or fragments thereof (such as those described elsewhere herein), and include immunoglobulins of other animal species, such as having humanized aspects. Single heavy-chain antibodies such as those identified in camelids are of particular interest in this regard. Novel frameworks, scaffolds and fragments continue to be discovered and developed by those skilled in the art.

In one aspect, the description pertains to generating non-immunoglobulin based antibodies using non-immunoglobulin scaffolds onto which CDRs described herein can be grafted. Known or future non-immunoglobulin frameworks and scaffolds may be employed, as long as they comprise a binding region specific for NPR1, e.g., such as those described for an antibody described herein including, but not limited to, N001 or N002. Such compounds are known herein as “polypeptides comprising a target-specific binding region”. Examples of non-immunoglobulin framework are further described in the sections below (camelid antibodies and non-antibody scaffold).

### Camelid Antibodies

Antibody proteins obtained from members of the camel and dromedary (Camelus bactrianus and Camelus dromaderius) family including new world members such as llama species (Lama paccos, Lama glama, and Lama vicugna) have been characterized with respect to size, structural complexity and antigenicity for human subjects. Certain IgG antibodies from this family of mammals as found in nature lack light chains, and are thus structurally distinct from the typical four chain quaternary structure having two heavy and two light chains, for antibodies from other animals, see WO94/04678, the contents of which are herein incorporated by reference for this purpose.

A region of the camelid antibody which is the small single variable domain identified as VHH can be obtained by genetic engineering to yield a small protein having high affinity for a target, resulting in a low molecular weight antibody-derived protein known as a “camelid nanobody”. See U.S. Pat. No. 5,759,808; see also Stijlemans, B. et al., 2004 J Biol Chem 279:1256-1261; Dumoulin, M. et al., 2003 Nature 424:783-788; Pleschberger, M. et al. 2003 Bioconjugate Chem 14:440-448; Cortez-Retamozo, V. et al. 2002 Int J Cancer 89:456-62; and Lauwereys, M. et al. 1998 EMBO J 17:3512-3520; the contents of each of which are herein incorporated by reference for this purpose. Engineered libraries of camelid antibodies and antibody fragments are commercially available, for example, from Ablynx, Ghent, Belgium. As with other antibodies of non-human origin, an amino acid sequence of a camelid antibody can be altered recombinantly to obtain a sequence that more closely resembles a human sequence, i.e., the nanobody can be “humanized”. Thus the natural low antigenicity of camelid antibodies to humans can be further reduced.

The camelid nanobody has a molecular weight approximately one-tenth that of a human IgG molecule, and the protein has a physical diameter of only a few nanometers. One consequence of the small size is the ability of camelid nanobodies to bind to antigenic sites that are functionally invisible to larger antibody proteins, i.e., camelid nanobodies are useful as reagents detect antigens that are otherwise cryptic using classical immunological techniques, and as possible therapeutic agents. Thus yet another consequence of small size is that a camelid nanobody can inhibit as a result of binding to a specific site in a groove or narrow cleft of a target protein, and hence can serve in a capacity that more closely resembles the function of a classical low molecular weight drug than that of a classical antibody.

The low molecular weight and compact size further result in camelid nanobodies being extremely thermostable, stable to extreme pH and to proteolytic digestion, and poorly antigenic. Another consequence is that camelid nanobodies readily move from the circulatory system into tissues, and even cross the blood-brain barrier and can treat disorders that affect nervous tissue. Nanobodies can further facilitated drug transport across the blood brain barrier, see US2004/0161738, the contents of which are herein incorporated by reference for this purpose. These features combined with the low antigenicity to humans indicate great therapeutic potential. Further, these molecules can be fully expressed in prokaryotic cells such as E. coli and may be expressed as functional fusion proteins with bacteriophage.

Accordingly, a feature of the present description is any of the uses or methods described herein, in which the subject is administered a camelid antibody or nanobody having high affinity for NPR1. In one embodiment, the camelid antibody or nanobody is obtained by grafting the CDRs sequences of the heavy or light chain of the human antibodies described herein into nanobody or single domain antibody framework sequences as described, for example, in WO94/04678 (the contents of which are herein incorporated by reference for this purpose).

### Framework or Fc Engineering

Engineered antibodies described herein include those in which modifications have been made to framework residues within VH and/or VL, e.g., to improve one or more properties of the antibody. Typically such framework modifications are made to decrease the immunogenicity of the antibody. Antibodies described herein may be modified in one or more ways, including each of the ways described herein.

For example, one approach is to “backmutate” one or more framework residues to the corresponding germline sequence. More specifically, an antibody that has undergone somatic mutation may contain framework residues that differ from the germline sequence from which the antibody is derived. Such residues can be identified by comparing the antibody framework sequences to the germline sequences from which the antibody is derived. To return the framework region sequences to their germline configuration, the somatic mutations can be “backmutated” to the germline sequence by, for example, site-directed mutagenesis or PCR-mediated mutagenesis. Such “backmutated” antibodies and additional modifications described herein are also intended to be encompassed by the methods and uses described herein.

Another type of framework modification involves mutating one or more residues within the framework region, or even within one or more CDR regions, to remove T-cell epitopes to thereby reduce the potential immunogenicity of the antibody. This approach is also referred to as “deimmunization” and is described in further detail in US2003/0153043, the contents of which are herein incorporated by reference for this purpose.

In addition or alternative to modifications made within the framework or CDR regions, antibodies described herein may be engineered to include modifications within the Fc region, typically to alter one or more functional properties of the antibody, such as serum half-life, complement fixation, Fc receptor binding, and/or antigen-dependent cellular cytotoxicity. Furthermore, an antibody described herein may be chemically modified (e.g., one or more chemical moieties can be attached to the antibody) or be modified to alter its glycosylation, again to alter one or more functional properties of the antibody. Each of these embodiments is described in further detail below. The numbering of residues in the Fc region is that of the EU index of Kabat.

In one embodiment, the hinge region of CH1 is modified such that the number of cysteine residues in the hinge region is altered, e.g., increased or decreased. This approach is described further in U.S. Pat. No. 5,677,425, the contents of which are herein incorporated by reference for this purpose. The number of cysteine residues in the hinge region of CH1 is altered to, for example, facilitate assembly of the light and heavy chains or to increase or decrease the stability of the antibody.

In another embodiment, the Fc hinge region of an antibody is mutated to decrease the biological half-life of the antibody. More specifically, one or more amino acid mutations are introduced into the CH2-CH3 domain interface region of the Fc-hinge fragment such that the antibody has impaired Staphylococcyl protein A (SpA) binding relative to native Fc-hinge domain SpA binding. This approach is described in further detail in U.S. Pat. No. 6,165,745, the contents of which are herein incorporated by reference for this purpose.

In another embodiment, the antibody is modified to increase its biological half-life. Various approaches are possible. For example, one or more of the following mutations can be introduced: T252L, T254S, T256F, as described in U.S. Pat. No. 6,277,375, the contents of which are herein incorporated by reference for this purpose. Alternatively, to increase the biological half life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022, the contents of each of which are herein incorporated by reference for this purpose.

In yet other embodiments, the Fc region is altered by replacing at least one amino acid residue with a different amino acid residue to alter the effector functions of the antibody. For example, one or more amino acids can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in further detail in U.S. Pat. Nos. 5,624,821 and 5,648,260, the contents of each of which are herein incorporated by reference for this purpose.

In order to minimize the ADCC activity of an antibody, specific mutations in the Fc region result in “Fc silent” antibodies that have minimal interaction with effector cells. In general, the “IgG Fc region” is used to define the C-terminal region of an immunoglobulin heavy chain, including native sequence Fc region and variant Fc regions. The human IgG heavy chain Fc region is generally defined as comprising the amino acid residue from position C226 or from P230 to the carboxyl-terminus of the IgG antibody. The numbering of residues in the Fc region is that of the EU index of Kabat. The C-terminal lysine (residue K447) of the Fc region may be removed, for example, during production or purification of the antibody.

Silenced effector functions can be obtained by mutation in the Fc region of the antibodies. See, for example, LALA and N297A (Strohl, W., 2009, Curr. Opin. Biotechnol. vol. 20 (6): 685-691); and D265A (Baudino et al., 2008, J. Immunol. 181:6664-69) see also Heusser et al., WO2012065950, the contents of each of which are herein incorporated by reference for this purpose. In particular, residues 234 and/or 235 may be mutated, optionally to alanine. Thus, in one embodiment, an antibody provided herein has a mutation in the Fc region at one or both of amino acids 234 and 235. Such substitution of both amino acids 234 and 235 results in reduced ADCC activity. One example of such a mutation is the LALA mutant comprising L234A and L235A mutation in the IgG1 Fc amino acid sequence. Another example of a silent IgG1 antibody is the DAPA (D265A, P329A) mutation (U.S. Pat. No. 6,737,056, the contents of which are herein incorporated by reference for this purpose). Another silent IgG1 antibody comprises the N297A mutation, which results in aglycosylated/non-glycosylated antibodies. Fc silent antibodies result in no or low ADCC activity, meaning that an Fc silent antibody exhibits an ADCC activity that is below 50% specific cell lysis. No ADCC activity means that the Fc silent antibody exhibits an ADCC activity (specific cell lysis) that is below 1%.

In another embodiment, one or more amino acids selected from amino acid residues can be replaced with a different amino acid residue such that the antibody has altered C1q binding and/or reduced or abolished complement dependent cytotoxicity (CDC). This approach is described in further detail in U.S. Pat. No. 6,194,551, the contents of which are herein incorporated by reference for this purpose.

In another embodiment, one or more amino acid residues are altered to thereby alter the ability of the antibody to fix complement. This approach is described further in WO94/29351, the contents of which are herein incorporated by reference for this purpose.

In yet another embodiment, the Fc region is modified to increase the ability of the antibody to mediate antibody dependent cellular cytotoxicity (ADCC) and/or to increase the affinity of the antibody for an Fcγ receptor by modifying one or more amino acids. This approach is described further in WO00/42072, the contents of which are herein incorporated by reference for this purpose. Moreover, the binding sites on human IgG1 for FcγRI, FcγRII, FcγRIII and FcRn have been mapped and variants with improved binding have been described (see Shields, R. L. et al., 2001 J. Biol. Chen. 276:6591-6604, the contents of which are herein incorporated by reference for this purpose).

In still another embodiment, the glycosylation of an antibody is modified. For example, an aglycoslated antibody can be made (i.e., the antibody lacks glycosylation). Glycosylation can be altered to, for example, increase the affinity of the antibody for “antigen”. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. Nos. 5,714,350 and 6,350,861, the contents of each of which are herein incorporated by reference for this purpose.

Additionally or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies described herein to thereby produce an antibody with altered glycosylation. For example, EP1176195 (the contents of which are herein incorporated by reference for this purpose) describes a cell line with a functionally disrupted FUT8 gene, which encodes a fucosyl transferase, such that antibodies expressed in such a cell line exhibit hypofucosylation. WO03/035835 describes a variant CHO cell line, Lec13 cells, with reduced ability to attach fucose to Asn (297)-linked carbohydrates, also resulting in hypofucosylation of antibodies expressed in that host cell (see also Shields, R. L. et al., 2002 J. Biol. Chem. 277:26733-26740). WO99/54342 describes cell lines engineered to express glycoprotein-modifying glycosyl transferases (e.g., beta (1,4)-N acetylglucosaminyltransferase III (GnTIII)) such that antibodies expressed in the engineered cell lines exhibit increased bisecting GlcNac structures which results in increased ADCC activity of the antibodies (see also Umana et al., 1999 Nat. Biotech. 17:176-180). The contents of each of the foregoing applications and references are herein incorporated by reference for this purpose

Another modification of the antibodies herein that is contemplated herein is pegylation. An antibody can be pegylated, for example, to increase the biological (e.g., serum) half-life of the antibody. To pegylate an antibody, the antibody, or fragment thereof, typically is reacted with polyethylene glycol (PEG), such as a reactive ester or aldehyde derivative of PEG, under conditions in which one or more PEG groups become attached to the antibody or antibody fragment. The pegylation can be carried out by an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C1-C10) alkoxy- or aryloxy-polyethylene glycol or polyethylene glycol-maleimide. In certain embodiments, the antibody to be pegylated is an aglycosylated antibody. Methods for pegylating proteins are known and can be applied to the antibodies described herein. See, for example, EP0154316 and EP0401384, the contents of each of which are herein incorporated by reference for this purpose.

Another modification of the antibodies that is contemplated is a conjugate or a protein fusion of at least the antigen-binding region of the antibody described herein to serum protein, such as human serum albumin or a fragment thereof to increase half-life of the resulting molecule. Such an approach is described, for example, in EP0322094, the contents of which are herein incorporated by reference for this purpose.

Another possibility is a fusion of at least the antigen-binding region of the antibody described herein to proteins capable of binding to serum proteins, such human serum albumin to increase half-life of the resulting molecule. Such approach is described, for example, in EP0486525, the contents of which are herein incorporated by reference for this purpose.

### Nucleic Acid Molecules Encoding Antibodies

Another aspect pertains to nucleic acid molecules that encode antibodies described herein. The term “nucleic acid” is used herein interchangeably with the term “polynucleotide,” and refers to deoxyribonucleotides or ribonucleotides and polymers thereof in either single- or double-stranded form. The term encompasses nucleic acids containing known nucleotide analogs or modified backbone residues or linkages, which are synthetic, naturally-occurring, and non-naturally-occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs). In some embodiments, the nucleic acid may be an mRNA.

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (See: Batzer et al., Nucleic Acids Res 1991; 25(19):5081; Ohtsuka et al., J Biol Chem 1985; 260(5):2605-8; Rossolini et al., Mol Cell Probes 1994; 8(2):91-8; the contents of each of which are herein incorporated by reference for this purpose).

Provided herein are exemplary full length heavy and light chain nucleotide sequences of anti-NPR1 antibodies for use in the methods and uses described herein. In some embodiments, the nucleic acid molecules are one or more of those identified in Table 2, e.g., those encoding an anti-NPR1 antibody or antigen binding fragment thereof. In some other embodiments, the nucleic acid molecules described herein comprise nucleotide sequences that are substantially identical (e.g., at least 80%, 85%, 90%, 91%, 92%, 93%, 94%, 95%, 96%, 97%, 98%, or 99%) to the nucleotide sequences of those identified in Table 2. When expressed from appropriate expression vectors, polypeptides encoded by these polynucleotides are capable of binding to a NPR1 protein (e.g., human NPR1).

Also provided herein are polynucleotides which encode at least one CDR region, and usually all three CDR regions, from the heavy and/or light chain of an anti-NPR1 antibody or antigen binding fragment described herein. Further provided herein are polynucleotides which encode all or substantially all of the variable region sequence of the heavy chain and/or the light chain of an exemplary anti-NPR1 antibody or antigen binding fragment described herein. Because of the degeneracy of the genetic code, a variety of nucleic acid sequences will encode each of the immunoglobulin amino acid sequences.

In some embodiments, the nucleic acid molecules described herein encode both a variable region and a constant region of an antibody. In some embodiments, the nucleic acid molecules described herein comprise nucleotides encoding a full-length heavy chain sequence that is substantially identical (e.g., at least 80%, 85%, 90%, 91%, 92%, 93%, 94%, 95%, 96%, 97%, 98%, or 99%) to the heavy chain sequence of one of the antibodies described herein including those in Table 2. In some embodiments, the nucleic acid molecules described herein comprise nucleotides encoding a full-length light chain sequence that is substantially identical (e.g., at least 80%, 85%, 90%, 91%, 92%, 93%, 94%, 95%, 96%, 97%, 98%, or 99%) to the light chain sequence of one of the antibodies described herein including those in Table 2.

The nucleic acids may be present in whole cells, in a cell lysate, or may be nucleic acids in a partially purified or substantially pure form. A nucleic acid is “isolated” or “rendered substantially pure” when purified away from other cellular components or other contaminants, e.g., other cellular nucleic acids or proteins, by standard techniques, including alkaline/SDS treatment, CsCl banding, column chromatography, agarose gel electrophoresis and others well known in the art. See, F. Ausubel, et al., ed. 1987 Current Protocols in Molecular Biology, Greene Publishing and Wiley Interscience, New York, the contents of which are herein incorporated by reference for this purpose. A nucleic acid described herein can be, for example, DNA or RNA and may or may not contain intronic sequences. In an embodiment, the nucleic acid is a cDNA molecule. The nucleic acid may be present in a vector such as a phage display vector, or in a recombinant plasmid vector.

Nucleic acids for the uses and methods as described herein can be obtained using standard molecular biology techniques. For antibodies expressed by hybridomas (e.g., hybridomas prepared from transgenic mice carrying human immunoglobulin genes as described further herein), cDNAs encoding the light and heavy chains of the antibody made by the hybridoma can be obtained by standard PCR amplification or cDNA cloning techniques. For antibodies obtained from an immunoglobulin gene library (e.g., using phage display techniques), nucleic acid encoding the antibody can be recovered from various phage clones that are members of the library.

The polynucleotide sequences can be produced by de novo solid-phase DNA synthesis or by PCR mutagenesis of an existing sequence (e.g., sequences as described herein in, for example, Table 2). Direct chemical synthesis of nucleic acids can be accomplished by any known method, such as the phosphotriester method of Narang et al., 1979, Meth. Enzymol. 68:90; the phosphodiester method of Brown et al., Meth. Enzymol. 68:109, 1979; the diethylphosphoramidite method of Beaucage et al., Tetra. Lett., 22:1859, 1981; and the solid support method of U.S. Pat. No. 4,458,066 (the contents of each of which are herein incorporated by reference for this purpose). Introducing mutations to a polynucleotide sequence by PCR can be performed as described in, e.g., PCR Technology: Principles and Applications for DNA Amplification, H. A. Erlich (Ed.), Freeman Press, NY, N.Y., 1992; PCR Protocols: A Guide to Methods and Applications, Innis et al. (Ed.), Academic Press, San Diego, Calif, 1990; Mattila et al., Nucleic Acids Res. 19:967, 1991; and Eckert et al., PCR Methods and Applications 1:17, 1991.

Once DNA fragments encoding VH and VL segments are obtained, these DNA fragments can be further manipulated by standard recombinant DNA techniques, for example to convert the variable region genes to full-length antibody chain genes, to Fab fragment genes or to an scFv gene. In these manipulations, a VL- or VH-encoding DNA fragment is operatively linked to another DNA molecule, or to a fragment encoding another protein, such as an antibody constant region or a flexible linker. The term “operatively linked”, as used in this context, is intended to mean that the two DNA fragments are joined in a functional manner, for example, such that the amino acid sequences encoded by the two DNA fragments remain in-frame, or such that the protein is expressed under control of a desired promoter.

The isolated DNA encoding the VH region can be converted to a full-length heavy chain gene by operatively linking the VH-encoding DNA to another DNA molecule encoding heavy chain constant regions (CH1, CH2 and CH3). The sequences of human heavy chain constant region genes are known (see e.g., Kabat, E. A., el al., 1991 Sequences of Proteins of Immunological Interest, Fifth Edition, U.S. Department of Health and Human Services, NIH Publication No. 91-3242, the contents of which are herein incorporated by reference for this purpose) and DNA fragments encompassing these regions can be obtained by standard PCR amplification. The heavy chain constant region can be an IgG1, IgG2, IgG3, IgG4, IgA, IgE, IgM, or IgD constant region. In some embodiments, the heavy chain constant region is an IgG1 isotype. For a Fab fragment heavy chain gene, the VH-encoding DNA can be operatively linked to another DNA molecule encoding only the heavy chain CH1 constant region.

The isolated DNA encoding the VL region can be converted to a full-length light chain gene (as well as to a Fab light chain gene) by operatively linking the VL-encoding DNA to another DNA molecule encoding the light chain constant region, CL. The sequences of human light chain constant region genes are known (see, e.g., Kabat, E. A., et al., 1991 Sequences of Proteins of Immunological Interest, Fifth Edition, U.S. Department of Health and Human Services, NIH Publication No. 91-3242, the contents of which are herein incorporated by reference for this purpose) and DNA fragments encompassing these regions can be obtained by standard PCR amplification. The light chain constant region can be a kappa or a lambda constant region.

To create an scFv gene, the VH- and VL-encoding DNA fragments are operatively linked to another fragment encoding a flexible linker, such that the VH and VL sequences can be expressed as a contiguous single-chain protein, with the VL and VH regions joined by the flexible linker (see e.g., Bird et al., 1988 Science 242:423-426; Huston et al., 1988 Proc. Natl. Acad. Sci. USA 85:5879-5883; McCafferty et al., 1990 Nature 348:552-554; the contents of each of which are herein incorporated by reference for this purpose).

### Vectors

Various expression vectors can be employed to express the polynucleotides encoding the antibody described herein or antigen-binding fragment thereof. Both viral-based and nonviral expression vectors can be used to produce the antibodies in a mammalian host cell. Nonviral vectors and systems include plasmids, episomal vectors, typically with an expression cassette for expressing a protein or RNA, and human artificial chromosomes (see, e.g., Harrington et al., Nat Genet. 15:345, 1997, the contents of which are herein incorporated by reference for this purpose). For example, nonviral vectors useful for expression of the polynucleotides and polypeptides of the multispecific antibody described herein or domains thereof in mammalian (e.g., human) cells include pThioHis A, B and C, pcDNA3.1/His, pEBVHis A, B and C, (Invitrogen, San Diego, Calif.), MPS V vectors, and numerous other known vectors for expressing other proteins. Useful viral vectors include vectors based on retroviruses, adenoviruses, adenoassociated viruses, herpes viruses, vectors based on SV40, papilloma virus, HBP Epstein Barr virus, vaccinia virus vectors and Semliki Forest virus (SFV). See, Brent et al., supra; Smith, Annu. Rev. Microbiol. 49:807, 1995; and Rosenfeld et al., Cell 68:143, 1992, the contents of each of which are herein incorporated by reference for this purpose.

The choice of expression vector depends on the intended host cells in which the vector is to be expressed. Expression vectors for mammalian host cells can include expression control sequences, such as an origin of replication, a promoter, and an enhancer (see, e.g., Queen et al., Immunol. Rev. 89:49-68, 1986, the contents of which are herein incorporated by reference for this purpose), and necessary processing information sites, such as ribosome binding sites, RNA splice sites, polyadenylation sites, and transcriptional terminator sequences. These expression vectors usually contain promoters derived from mammalian genes or from mammalian viruses. Suitable promoters may be constitutive, cell type-specific, stage-specific, and/or modulatable or regulatable. Useful promoters include, but are not limited to, the metallothionein promoter, the constitutive adenovirus major late promoter, the dexamethasone-inducible MMTV promoter, the SV40 promoter, the MRP poIIII promoter, the constitutive MPS V promoter, the tetracycline-inducible CMV promoter (such as the human immediate-early CMV promoter), the constitutive CMV promoter, and known promoter-enhancer combinations.

Cultures of transformed organisms can be expanded under non-inducing conditions without biasing the population for coding sequences whose expression products are better tolerated by the host cells. In addition to promoters, other regulatory elements may also be required or desired for efficient expression of the antibody described herein or fragments thereof. These elements typically include an ATG initiation codon and adjacent ribosome binding site or other sequences. In addition, the efficiency of expression may be enhanced by the inclusion of enhancers appropriate to the cell system in use (see, e.g., Scharf et al., Results Probl. Cell Differ. 20:125, 1994; and Bittner et al., Meth. Enzymol., 153:516, 1987; the contents of each of which are herein incorporated by reference for this purpose). For example, the SV40 enhancer or CMV enhancer may be used to increase expression in mammalian host cells.

Accordingly, provided for any of the uses or methods described herein is a cloning or expression vector comprising one or more of the nucleic acid sequences of the antibodies shown in Table 2. Furthermore, provided for any of the uses or methods described herein is a cloning or expression vector comprising a nucleic acid encoding one or more of the nucleotide sequences shown in Table 2.

### Host Cells

For expression of the light and heavy chains, the expression vector or expression vectors encoding the heavy and light chains may be transferred into a host cell by standard techniques.

Methods for introducing expression vectors containing the polynucleotide sequences of interest vary depending on the type of cellular host. For example, calcium chloride transfection is commonly utilized for prokaryotic cells, whereas calcium phosphate treatment or electroporation may be used for other cellular hosts. (See generally Sambrook, et al., supra, the contents of which are herein incorporated by reference for this purpose). Other methods include, e.g., electroporation, calcium phosphate treatment, liposome-mediated transformation, injection and microinjection, ballistic methods, virosomes, immunoliposomes, polycatiomnucleic acid conjugates, naked DNA, artificial virions, fusion to the herpes virus structural protein VP22 (Elliot and O'Hare, Cell 88:223, 1997, the contents of which are herein incorporated by reference for this purpose), agent-enhanced uptake of DNA, and ex vivo transduction.

It is theoretically possible to express the antibodies described herein in either prokaryotic or eukaryotic host cells. Expression of antibodies in eukaryotic cells, in particular mammalian host cells, is discussed because such eukaryotic cells, and in particular mammalian cells, are more likely than prokaryotic cells to assemble and secrete a properly folded and immunologically active antibody. Prokaryotic expression of antibody genes has been reported to be ineffective for production of high yields of active antibody (Boss, M. A. and Wood, C. R., 1985 Immunology Today 6:12-13, the contents of which are herein incorporated by reference for this purpose).

For long-term, high-yield production of recombinant proteins, stable expression will often be desired. For example, cell lines which stably express the antibodies or antigen-binding fragments thereof described herein can be prepared using expression vectors described herein which contain viral origins of replication or endogenous expression elements and a selectable marker gene. Following the introduction of the vector, cells may be allowed to grow for 1-2 days in an enriched media before they are switched to selective media. The purpose of the selectable marker is to confer resistance to selection, and its presence allows growth of cells which successfully express the introduced sequences in selective media. Resistant, stably transfected cells can be proliferated using tissue culture techniques appropriate to the cell type. Also provided is a method of producing the antibodies or antigen-binding fragments described herein, wherein said method comprises the step of culturing a host cell comprising a nucleic acid encoding the antibodies or antigen-binding fragments.

In some embodiments, mammalian host cells are used to express and produce the anti-NPR1 antibodies or antigen binding fragments described herein for any of the uses or methods described herein. For example, they can be either a hybridoma cell line expressing endogenous immunoglobulin genes or a mammalian cell line harboring an exogenous expression vector. These include any normal mortal or normal or abnormal immortal animal or human cell. For example, a number of suitable host cell lines capable of secreting intact immunoglobulins have been developed including the CHO cell lines, various COS cell lines, HeLa cells, myeloma cell lines, transformed B-cells, and hybridomas. Exemplary host cells include but are not limited to Chinese hamster ovary (CHO) cells, human embryonic kidney (HEK) cells (e.g., HEK293, HEK293T, HEK293F), monkey kidney (COS) cells (e.g., COS-1, COS-7), baby hamster kidney (BHK) cells (e.g., BHK-21), African green monkey kidney cells (e.g. BSC-1), HeLa cells, human hepatocellular carcinoma cells (e.g., Hep G2), myeloma cells (e.g., NS0, 653, SP2/0), lymphoma cells, oocyte cells, and cells from a transgenic animal (e.g., mammary epithelial cells), or any derivative, immortalized, or transformed cell thereof. In particular, for use with NS0 myeloma cells, another expression system is the GS gene expression system shown in WO87/04462, WO89/01036 and EP0338841, the contents of each of which are herein incorporated by reference for this purpose. When recombinant expression vectors encoding antibody nucleic acid are introduced into mammalian host cells, the antibodies are produced by culturing the host cells for a period of time sufficient to allow for expression of the antibody in the host cells or secretion of the antibody into the culture medium in which the host cells are grown. Antibodies can be recovered from the culture medium using standard protein purification methods. Such purified antibodies described herein may be used for any purpose including, but not limited to, the methods and uses described herein, and/or as part of a pharmaceutical composition as described herein.

In a further alternative, the host cell may be a yeast or a filamentous fungi engineered for mammalian-like glycosylation pattern, and capable for producing antibodies lacking fucose as glycosylation pattern (see, for example, EP1297172, the contents of which are herein incorporated by reference for this purpose).

Accordingly, provided for any of the uses or methods described herein is a host cell comprising one or more of the vectors, or nucleic acid sequences described herein described above.

### Uses and Methods of Treatment

### Therapeutic Methods and Pharmaceutical Compositions for Use

Provided herein are methods and pharmaceutical compositions for use in treating a disease associated with NPR1 loss of function by using the anti-NPR1 antibodies or antigen binding fragments thereof described herein (e.g., an antibody or group of antibodies as defined in Table 2). In some embodiments, the antibody or antigen binding fragment thereof may be selected from N001 (e.g., N001_DAPA or N001_LALA) and N002 (e.g., N002_DAPA or N002_LALA).

Provided herein are uses of the anti-NPR1 antibodies or antigen binding fragments thereof described herein (e.g., an antibody or group of antibodies as defined in Table 2) and pharmaceutical composition(s) thereof for the treatment of a disease associated with NPR1 loss of function. In some embodiments, the antibody or antigen binding fragment thereof may be selected from N001 (e.g., N001_DAPA or N001_LALA) and N002 (e.g., N002_DAPA or N002_LALA).

Provided herein are anti-NPR1 antibodies or antigen binding fragments thereof (e.g., an antibody or group of antibodies as defined in Table 2) for the manufacture of a medicament for the treatment of a disease associated with NPR1 loss of function. In some embodiments, the antibody or antigen binding fragment thereof may be selected from N001 (e.g., N001_DAPA or N001_LALA) and N002 (e.g., N002_DAPA or N002_LALA).

In some embodiments, the antibody or antigen binding fragment thereof may be selected from N001 (e.g., N001_DAPA or N001_LALA) and N002 (e.g., N002_DAPA or N002_LALA). In some embodiments, the antibody or antigen binding fragment thereof may be selected from N001_DAPA. In some embodiments, the antibody or antigen binding fragment thereof may be N001_LALA. In some embodiments, the antibody or antigen binding fragment thereof may be N002_DAPA. In some embodiments, the antibody or antigen binding fragment thereof may be N002_LALA.

In some embodiments, the disease associated with NPR1 loss of function is a cardiovascular disorder. In some embodiments, the cardiovascular disorder is selected from: hypertension, peripheral vascular disease, heart failure, coronary artery disease (CAD), ischemic heart disease (IHD), mitral stenosis and regurgitation, angina, hypertrophic cardiomyopathy, diabetic cardiomyopathy, supraventricular and ventricular arrhythmias, cardiac dysrhythmia, atrial fibrillation (AF), new onset of atrial fibrillation, recurrent atrial fibrillation, cardiac fibrosis, atrial flutter, detrimental vascular remodeling, plaque stabilization, and myocardial infarction (MI). In some embodiments, the disease associated with NPR1 loss of function is heart failure, hypertrophic cardiomyopathy (HCM), hypertension, preeclampsia, asthma, glaucoma, or cytokine release syndrome. In some embodiments, the heart failure is selected from a heart failure with reduced ejection fraction (HFrEF), heart failure with mid-range ejection fraction or heart failure with mildly reduced ejection fraction or heart failure with mildly reduced ejection fraction (HFmrEF), heart failure with preserved ejection fraction (HFpEF), heart failure after acute myocardial infarct, or acute decompensated heart failure.

In some embodiments, the hypertrophic cardiomyopathy is ventricular hypertrophy. In some embodiments, the hypertension is selected from resistant hypertension, hypertensive heart disease, pulmonary hypertension, isolated systolic hypertension, and pulmonary arterial hypertension. In some embodiments, the hypertension is selected from resistant hypertension and hypertensive heart disease.

In some embodiments, the disease associated with NPR1 loss of function is a kidney disorder. In some embodiments, the kidney disorder is selected from: diabetic renal insufficiency, non-diabetic renal insufficiency, renal failure, diabetic nephropathy, non-diabetic nephropathy, acute renal injury, contrast induced nephropathy, nephrotic syndrome, glomerulonephritis, scleroderma, glomerular sclerosis, proteinuria of primary renal disease, renal vascular hypertension, diabetic retinopathy and end-stage renal disease (ESRD), endothelial dysfunction, diastolic dysfunction, renal fibrosis, and polycystic kidney disease (PKD).

NPR1-related disorders also include any other disorders which are directly or indirectly associated with aberrant NPR1 activity and/or expression. Provided herein are also methods of treating a NPR1 related disorder directly or indirectly associated with aberrant NPR1 activity and/or expression by using the anti-NPR1 antibodies or antigen binding fragments described herein (e.g., from Table 2, such as N001_DAPA or N001_LALA).

In some embodiments, provided are methods of treating an undesirable condition, disease, or disorder associated with natriuretic peptide receptor activity in a subject in need thereof, the method comprising administering to the subject a therapeutically effective amount of an antibody or antigen binding fragment described herein. In some embodiments, provided is a use of an antibody or antigen binding fragment described herein for treatment of an undesirable condition, disease or disorder associated with natriuretic peptide receptor activity in a subject in need thereof. In some embodiments, provided is an antibody or antigen binding fragment described herein for use in a method for treating an undesirable condition, disease or disorder associated with natriuretic peptide receptor activity. In some embodiments, provided is an antibody or antigen binding fragment described herein for use in manufacturing a medicament for treating an undesirable condition, disease or disorder associated with natriuretic peptide receptor activity. Such conditions, diseases and disorders include, but are not limited to, cardiovascular disorders (e.g., hypertension, peripheral vascular disease, heart failure (including but not limited to heart failure with reduced ejection fraction (HFrEF), heart failure with mid-range ejection fraction or heart failure with mildly reduced ejection fraction or heart failure with mildly reduced ejection fraction (HFmrEF), heart failure with preserved ejection fraction (HFpEF), heart failure after acute myocardial infarct, or acute decompensated heart failure), coronary artery disease (CAD), ischemic heart disease (IHD), mitral stenosis and regurgitation, angina, hypertrophic cardiomyopathy (e.g., ventricular hypertrophy), diabetic cardiomyopathy, supraventricular and ventricular arrhythmias, cardiac dysrhythmia, atrial fibrillation (AF), new onset of atrial fibrillation, recurrent atrial fibrillation, cardiac fibrosis, atrial flutter, detrimental vascular remodeling, plaque stabilization, or myocardial infarction (MI)), hypertension (e.g., resistant hypertension, hypertensive heart disease, pulmonary hypertension, pulmonary arterial hypertension, isolated systolic hypertension, resistant hypertension, or pulmonary arterial hypertension), preeclampsia, asthma, glaucoma, cytokine release syndrome, and/or a kidney disorder (e.g., diabetic renal insufficiency, non-diabetic renal insufficiency, renal failure, diabetic nephropathy, non-diabetic nephropathy, acute renal injury, contrast induced nephropathy, nephrotic syndrome, glomerulonephritis, scleroderma, glomerular sclerosis, proteinuria of primary renal disease, renal vascular hypertension, diabetic retinopathy and end-stage renal disease (ESRD), endothelial dysfunction, diastolic dysfunction, renal fibrosis, and polycystic kidney disease (PKD)).

In some aspects, provided herein are methods of treating a disorder or a disease associated with natriuretic peptide receptor 1 (NPR1) activity (e.g., a cardiovascular disorder) in a subject in need thereof, the method comprising administering to the subject about 10 mg, about 15 mg, about 20 mg, about 25 mg, about 30 mg, about 35 mg, about 40 mg, about 45 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 350 mg, about 400 mg, about 450 mg, about 500 mg, about 550 mg, about 600 mg, about 650 mg, or about 700 mg of an anti-NPR1 antibody (e.g., N001_DAPA or N001_LALA) or antigen binding fragment thereof. In some embodiments, the methods of treating a disorder or a disease associated with natriuretic peptide receptor 1 (NPR1) activity (e.g., a cardiovascular disorder) comprises administering about 60 mg of an anti-NPR1 antibody (e.g., N001_DAPA or N001_LALA) or antigen binding fragment thereof. In some embodiments, the methods of treating a disorder or a disease associated with natriuretic peptide receptor 1 (NPR1) activity (e.g., a cardiovascular disorder) comprises administering about 120 mg of an anti-NPR1 antibody (e.g., N001_DAPA or N001_LALA) or antigen binding fragment thereof. In some embodiments, the methods of treating a disorder or a disease associated with natriuretic peptide receptor 1 (NPR1) activity (e.g., a cardiovascular disorder) comprises administering about 240 mg of an anti-NPR1 antibody (e.g., N001_DAPA or N001_LALA) or antigen binding fragment thereof. In some aspects, provided herein are methods of treating a cardiovascular disorder in a subject in need thereof, the method comprising administering to the subject about 10 mg, about 15 mg, about 20 mg, about 25 mg, about 30 mg, about 35 mg, about 40 mg, about 45 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 350 mg, about 400 mg, about 450 mg, about 500 mg, about 550 mg, about 600 mg, about 650 mg, or about 700 mg of an anti-NPR1 antibody (e.g., N001_DAPA or N001_LALA) or antigen binding fragment thereof. In some embodiments, the methods of treating a cardiovascular disorder in a subject in need thereof comprise administering to the subject about comprises administering about 60 mg of an anti-NPR1 antibody (e.g., N001_DAPA or N001_LALA) or antigen binding fragment thereof. In some embodiments, the methods of treating a cardiovascular disorder in a subject in need thereof comprise administering to the subject about comprises administering about 120 mg of an anti-NPR1 antibody (e.g., N001_DAPA or N001_LALA) or antigen binding fragment thereof. In some embodiments, the methods of treating a cardiovascular disorder in a subject in need thereof comprise administering about 240 mg of an anti-NPR1 antibody (e.g., N001_DAPA or N001_LALA) or antigen binding fragment thereof. In some aspects, provided herein are methods of treating a disorder or a disease associated with natriuretic peptide receptor 1 (NPR1) activity (e.g., preeclampsia, asthma, glaucoma, cytokine release syndrome or a kidney disorder such as diabetic renal insufficiency, non-diabetic renal insufficiency, renal failure, diabetic nephropathy, non-diabetic nephropathy, acute renal injury, contrast induced nephropathy, nephrotic syndrome, glomerulonephritis, scleroderma, glomerular sclerosis, proteinuria of primary renal disease, renal vascular hypertension, diabetic retinopathy and end-stage renal disease (ESRD), endothelial dysfunction, diastolic dysfunction, renal fibrosis, and polycystic kidney disease (PKD)) in a subject in need thereof, the method comprising administering to the subject about 10 mg, about 15 mg, about 20 mg, about 25 mg, about 30 mg, about 35 mg, about 40 mg, about 45 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 350 mg, about 400 mg, about 450 mg, about 500 mg, about 550 mg, about 600 mg, about 650 mg, or about 700 mg of an anti-NPR1 antibody (e.g., N001_DAPA or N001_LALA) or antigen binding fragment thereof. In some embodiments, the methods of treating a disorder or a disease associated with natriuretic peptide receptor 1 (NPR1) activity (e.g., preeclampsia, asthma, glaucoma, cytokine release syndrome or a kidney disorder such as diabetic renal insufficiency, non-diabetic renal insufficiency, renal failure, diabetic nephropathy, non-diabetic nephropathy, acute renal injury, contrast induced nephropathy, nephrotic syndrome, glomerulonephritis, scleroderma, glomerular sclerosis, proteinuria of primary renal disease, renal vascular hypertension, diabetic retinopathy and end-stage renal disease (ESRD), endothelial dysfunction, diastolic dysfunction, renal fibrosis, and polycystic kidney disease (PKD)) comprises administering about 60 mg of an anti-NPR1 antibody (e.g., N001_DAPA or N001_LALA) or antigen binding fragment thereof. In some embodiments, the methods of treating a disorder or a disease associated with natriuretic peptide receptor 1 (NPR1) activity (e.g., preeclampsia, asthma, glaucoma, cytokine release syndrome or a kidney disorder such as diabetic renal insufficiency, non-diabetic renal insufficiency, renal failure, diabetic nephropathy, non-diabetic nephropathy, acute renal injury, contrast induced nephropathy, nephrotic syndrome, glomerulonephritis, scleroderma, glomerular sclerosis, proteinuria of primary renal disease, renal vascular hypertension, diabetic retinopathy and end-stage renal disease (ESRD), endothelial dysfunction, diastolic dysfunction, renal fibrosis, and polycystic kidney disease (PKD)) comprises administering about 120 mg of an anti-NPR1 antibody (e.g., N001_DAPA or N001_LALA) or antigen binding fragment thereof. In some embodiments, the methods of treating a disorder or a disease associated with natriuretic peptide receptor 1 (NPR1) activity (e.g., preeclampsia, asthma, glaucoma, cytokine release syndrome or a kidney disorder such as diabetic renal insufficiency, non-diabetic renal insufficiency, renal failure, diabetic nephropathy, non-diabetic nephropathy, acute renal injury, contrast induced nephropathy, nephrotic syndrome, glomerulonephritis, scleroderma, glomerular sclerosis, proteinuria of primary renal disease, renal vascular hypertension, diabetic retinopathy and end-stage renal disease (ESRD), endothelial dysfunction, diastolic dysfunction, renal fibrosis, and polycystic kidney disease (PKD)) comprises administering about 240 mg of an anti-NPR1 antibody (e.g., N001_DAPA or N001_LALA) or antigen binding fragment thereof. In some embodiments, provided herein are methods of treating hypertension, peripheral vascular disease, heart failure, coronary artery disease (CAD), ischemic heart disease (IHD), mitral stenosis and regurgitation, angina, hypertrophic cardiomyopathy, diabetic cardiomyopathy, supraventricular and ventricular arrhythmias, cardiac dysrhythmia, atrial fibrillation (AF), new onset of atrial fibrillation, recurrent atrial fibrillation, cardiac fibrosis, atrial flutter, detrimental vascular remodeling, plaque stabilization, or myocardial infarction (MI) in a subject in need thereof, the method comprising administering to the subject about 60 mg of an anti-NPR1 antibody (e.g., N001_DAPA or N001_LALA) or antigen binding fragment thereof. In some embodiments, provided herein are methods of treating hypertension, peripheral vascular disease, heart failure, coronary artery disease (CAD), ischemic heart disease (IHD), mitral stenosis and regurgitation, angina, hypertrophic cardiomyopathy, diabetic cardiomyopathy, supraventricular and ventricular arrhythmias, cardiac dysrhythmia, atrial fibrillation (AF), new onset of atrial fibrillation, recurrent atrial fibrillation, cardiac fibrosis, atrial flutter, detrimental vascular remodeling, plaque stabilization, or myocardial infarction (MI) in a subject in need thereof, the method comprising administering to the subject about 70 mg of an anti-NPR1 antibody (e.g., N001_DAPA or N001_LALA) or antigen binding fragment thereof. In some embodiments, provided herein are methods of treating hypertension, peripheral vascular disease, heart failure, coronary artery disease (CAD), ischemic heart disease (IHD), mitral stenosis and regurgitation, angina, hypertrophic cardiomyopathy, diabetic cardiomyopathy, supraventricular and ventricular arrhythmias, cardiac dysrhythmia, atrial fibrillation (AF), new onset of atrial fibrillation, recurrent atrial fibrillation, cardiac fibrosis, atrial flutter, detrimental vascular remodeling, plaque stabilization, or myocardial infarction (MI) in a subject in need thereof, the method comprising administering to the subject about 80 mg of an anti-NPR1 antibody (e.g., N001_DAPA or N001_LALA) or antigen binding fragment thereof. In some embodiments, provided herein are methods of treating hypertension, peripheral vascular disease, heart failure, coronary artery disease (CAD), ischemic heart disease (IHD), mitral stenosis and regurgitation, angina, hypertrophic cardiomyopathy, diabetic cardiomyopathy, supraventricular and ventricular arrhythmias, cardiac dysrhythmia, atrial fibrillation (AF), new onset of atrial fibrillation, recurrent atrial fibrillation, cardiac fibrosis, atrial flutter, detrimental vascular remodeling, plaque stabilization, or myocardial infarction (MI) in a subject in need thereof, the method comprising administering to the subject about 90 mg of an anti-NPR1 antibody (e.g., N001_DAPA or N001_LALA) or antigen binding fragment thereof. In some embodiments, provided herein are methods of treating hypertension, peripheral vascular disease, heart failure, coronary artery disease (CAD), ischemic heart disease (IHD), mitral stenosis and regurgitation, angina, hypertrophic cardiomyopathy, diabetic cardiomyopathy, supraventricular and ventricular arrhythmias, cardiac dysrhythmia, atrial fibrillation (AF), new onset of atrial fibrillation, recurrent atrial fibrillation, cardiac fibrosis, atrial flutter, detrimental vascular remodeling, plaque stabilization, or myocardial infarction (MI) in a subject in need thereof, the method comprising administering to the subject about 100 mg of an anti-NPR1 antibody (e.g., N001_DAPA or N001_LALA) or antigen binding fragment thereof. In some embodiments, provided herein are methods of treating hypertension, peripheral vascular disease, heart failure, coronary artery disease (CAD), ischemic heart disease (IHD), mitral stenosis and regurgitation, angina, hypertrophic cardiomyopathy, diabetic cardiomyopathy, supraventricular and ventricular arrhythmias, cardiac dysrhythmia, atrial fibrillation (AF), new onset of atrial fibrillation, recurrent atrial fibrillation, cardiac fibrosis, atrial flutter, detrimental vascular remodeling, plaque stabilization, or myocardial infarction (MI) in a subject in need thereof, the method comprising administering to the subject about 110 mg of an anti-NPR1 antibody (e.g., N001_DAPA or N001_LALA) or antigen binding fragment thereof. In some embodiments, provided herein are methods of treating hypertension, peripheral vascular disease, heart failure, coronary artery disease (CAD), ischemic heart disease (IHD), mitral stenosis and regurgitation, angina, hypertrophic cardiomyopathy, diabetic cardiomyopathy, supraventricular and ventricular arrhythmias, cardiac dysrhythmia, atrial fibrillation (AF), new onset of atrial fibrillation, recurrent atrial fibrillation, cardiac fibrosis, atrial flutter, detrimental vascular remodeling, plaque stabilization, or myocardial infarction (MI) in a subject in need thereof, the method comprising administering to the subject about 120 mg of an anti-NPR1 antibody (e.g., N001_DAPA or N001_LALA) or antigen binding fragment thereof. In some embodiments, provided herein are methods of treating hypertension, peripheral vascular disease, heart failure, coronary artery disease (CAD), ischemic heart disease (IHD), mitral stenosis and regurgitation, angina, hypertrophic cardiomyopathy, diabetic cardiomyopathy, supraventricular and ventricular arrhythmias, cardiac dysrhythmia, atrial fibrillation (AF), new onset of atrial fibrillation, recurrent atrial fibrillation, cardiac fibrosis, atrial flutter, detrimental vascular remodeling, plaque stabilization, or myocardial infarction (MI) in a subject in need thereof, the method comprising administering to the subject about 130 mg of an anti-NPR1 antibody (e.g., N001_DAPA or N001_LALA) or antigen binding fragment thereof. In some embodiments, provided herein are methods of treating hypertension, peripheral vascular disease, heart failure, coronary artery disease (CAD), ischemic heart disease (IHD), mitral stenosis and regurgitation, angina, hypertrophic cardiomyopathy, diabetic cardiomyopathy, supraventricular and ventricular arrhythmias, cardiac dysrhythmia, atrial fibrillation (AF), new onset of atrial fibrillation, recurrent atrial fibrillation, cardiac fibrosis, atrial flutter, detrimental vascular remodeling, plaque stabilization, or myocardial infarction (MI) in a subject in need thereof, the method comprising administering to the subject about 140 mg of an anti-NPR1 antibody (e.g., N001_DAPA or N001_LALA) or antigen binding fragment thereof. In some embodiments, provided herein are methods of treating hypertension, peripheral vascular disease, heart failure, coronary artery disease (CAD), ischemic heart disease (IHD), mitral stenosis and regurgitation, angina, hypertrophic cardiomyopathy, diabetic cardiomyopathy, supraventricular and ventricular arrhythmias, cardiac dysrhythmia, atrial fibrillation (AF), new onset of atrial fibrillation, recurrent atrial fibrillation, cardiac fibrosis, atrial flutter, detrimental vascular remodeling, plaque stabilization, or myocardial infarction (MI) in a subject in need thereof, the method comprising administering to the subject about 150 mg of an anti-NPR1 antibody (e.g., N001_DAPA or N001_LALA) or antigen binding fragment thereof. In some embodiments, provided herein are methods of treating hypertension, peripheral vascular disease, heart failure, coronary artery disease (CAD), ischemic heart disease (IHD), mitral stenosis and regurgitation, angina, hypertrophic cardiomyopathy, diabetic cardiomyopathy, supraventricular and ventricular arrhythmias, cardiac dysrhythmia, atrial fibrillation (AF), new onset of atrial fibrillation, recurrent atrial fibrillation, cardiac fibrosis, atrial flutter, detrimental vascular remodeling, plaque stabilization, or myocardial infarction (MI) in a subject in need thereof, the method comprising administering to the subject about 160 mg of an anti-NPR1 antibody (e.g., N001_DAPA or N001_LALA) or antigen binding fragment thereof. In some embodiments, provided herein are methods of treating hypertension, peripheral vascular disease, heart failure, coronary artery disease (CAD), ischemic heart disease (IHD), mitral stenosis and regurgitation, angina, hypertrophic cardiomyopathy, diabetic cardiomyopathy, supraventricular and ventricular arrhythmias, cardiac dysrhythmia, atrial fibrillation (AF), new onset of atrial fibrillation, recurrent atrial fibrillation, cardiac fibrosis, atrial flutter, detrimental vascular remodeling, plaque stabilization, or myocardial infarction (MI) in a subject in need thereof, the method comprising administering to the subject about 170 mg of an anti-NPR1 antibody (e.g., N001_DAPA or N001_LALA) or antigen binding fragment thereof. In some embodiments, provided herein are methods of treating hypertension, peripheral vascular disease, heart failure, coronary artery disease (CAD), ischemic heart disease (IHD), mitral stenosis and regurgitation, angina, hypertrophic cardiomyopathy, diabetic cardiomyopathy, supraventricular and ventricular arrhythmias, cardiac dysrhythmia, atrial fibrillation (AF), new onset of atrial fibrillation, recurrent atrial fibrillation, cardiac fibrosis, atrial flutter, detrimental vascular remodeling, plaque stabilization, or myocardial infarction (MI) in a subject in need thereof, the method comprising administering to the subject about 180 mg of an anti-NPR1 antibody (e.g., N001_DAPA or N001_LALA) or antigen binding fragment thereof. In some embodiments, provided herein are methods of treating hypertension, peripheral vascular disease, heart failure, coronary artery disease (CAD), ischemic heart disease (IHD), mitral stenosis and regurgitation, angina, hypertrophic cardiomyopathy, diabetic cardiomyopathy, supraventricular and ventricular arrhythmias, cardiac dysrhythmia, atrial fibrillation (AF), new onset of atrial fibrillation, recurrent atrial fibrillation, cardiac fibrosis, atrial flutter, detrimental vascular remodeling, plaque stabilization, or myocardial infarction (MI) in a subject in need thereof, the method comprising administering to the subject about 190 mg of an anti-NPR1 antibody (e.g., N001_DAPA or N001_LALA) or antigen binding fragment thereof. In some embodiments, provided herein are methods of treating hypertension, peripheral vascular disease, heart failure, coronary artery disease (CAD), ischemic heart disease (IHD), mitral stenosis and regurgitation, angina, hypertrophic cardiomyopathy, diabetic cardiomyopathy, supraventricular and ventricular arrhythmias, cardiac dysrhythmia, atrial fibrillation (AF), new onset of atrial fibrillation, recurrent atrial fibrillation, cardiac fibrosis, atrial flutter, detrimental vascular remodeling, plaque stabilization, or myocardial infarction (MI) in a subject in need thereof, the method comprising administering to the subject about 200 mg of an anti-NPR1 antibody (e.g., N001_DAPA or N001_LALA) or antigen binding fragment thereof.

In some aspects, provided herein are methods of treating a disorder or a disease associated with natriuretic peptide receptor 1 (NPR1) activity (e.g., a cardiovascular disorder) in a subject in need thereof, the method comprising administering to the subject about 10 mg, about 15 mg, about 20 mg, about 25 mg, about 30 mg, about 35 mg, about 40 mg, about 45 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 350 mg, about 400 mg, about 450 mg, about 500 mg, about 550 mg, about 600 mg, about 650 mg, or about 700 mg of an anti-NPR1 antibody (e.g., N001_DAPA or N001_LALA) or antigen binding fragment thereof. In some embodiments, the methods of treating a disorder or a disease associated with natriuretic peptide receptor 1 (NPR1) activity (e.g., a cardiovascular disorder) comprises administering about 60 mg of an anti-NPR1 antibody (e.g., N001_DAPA or N001_LALA) or antigen binding fragment thereof. In some embodiments, the methods of treating a disorder or a disease associated with natriuretic peptide receptor 1 (NPR1) activity (e.g., a cardiovascular disorder) comprises administering about 120 mg of an anti-NPR1 antibody (e.g., N001_DAPA or N001_LALA) or antigen binding fragment thereof. In some embodiments, the methods of treating a disorder or a disease associated with natriuretic peptide receptor 1 (NPR1) activity (e.g., a cardiovascular disorder) comprises administering about 240 mg of an anti-NPR1 antibody (e.g., N001_DAPA or N001_LALA) or antigen binding fragment thereof.

In some aspects, provided herein are methods of treating a cardiovascular disorder in a subject in need thereof, the method comprising administering to the subject about 10 mg, about 15 mg, about 20 mg, about 25 mg, about 30 mg, about 35 mg, about 40 mg, about 45 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 350 mg, about 400 mg, about 450 mg, about 500 mg, about 550 mg, about 600 mg, about 650 mg, or about 700 mg of an anti-NPR1 antibody (e.g., N001_DAPA or N001_LALA) or antigen binding fragment thereof. In some embodiments, the methods of treating a cardiovascular disorder in a subject in need thereof comprise administering to the subject about comprises administering about 60 mg of an anti-NPR1 antibody (e.g., N001_DAPA or N001_LALA) or antigen binding fragment thereof. In some embodiments, the methods of treating a cardiovascular disorder in a subject in need thereof comprise administering to the subject about comprises administering about 120 mg of an anti-NPR1 antibody (e.g., N001_DAPA or N001_LALA) or antigen binding fragment thereof. In some embodiments, the methods of treating a cardiovascular disorder in a subject in need thereof comprise administering about 240 mg of an anti-NPR1 antibody (e.g., N001_DAPA or N001_LALA) or antigen binding fragment thereof.

In some aspects, provided herein are methods of treating a disorder or a disease associated with natriuretic peptide receptor 1 (NPR1) activity (e.g., preeclampsia, asthma, glaucoma, cytokine release syndrome or a kidney disorder such as diabetic renal insufficiency, non-diabetic renal insufficiency, renal failure, diabetic nephropathy, non-diabetic nephropathy, acute renal injury, contrast induced nephropathy, nephrotic syndrome, glomerulonephritis, scleroderma, glomerular sclerosis, proteinuria of primary renal disease, renal vascular hypertension, diabetic retinopathy and end-stage renal disease (ESRD), endothelial dysfunction, diastolic dysfunction, renal fibrosis, and polycystic kidney disease (PKD)) in a subject in need thereof, the method comprising administering to the subject about 10 mg, about 15 mg, about 20 mg, about 25 mg, about 30 mg, about 35 mg, about 40 mg, about 45 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 350 mg, about 400 mg, about 450 mg, about 500 mg, about 550 mg, about 600 mg, about 650 mg, or about 700 mg of an anti-NPR1 antibody (e.g., N001_DAPA or N001_LALA) or antigen binding fragment thereof. In some embodiments, the methods of treating a disorder or a disease associated with natriuretic peptide receptor 1 (NPR1) activity (e.g., preeclampsia, asthma, glaucoma, cytokine release syndrome or a kidney disorder such as diabetic renal insufficiency, non-diabetic renal insufficiency, renal failure, diabetic nephropathy, non-diabetic nephropathy, acute renal injury, contrast induced nephropathy, nephrotic syndrome, glomerulonephritis, scleroderma, glomerular sclerosis, proteinuria of primary renal disease, renal vascular hypertension, diabetic retinopathy and end-stage renal disease (ESRD), endothelial dysfunction, diastolic dysfunction, renal fibrosis, and polycystic kidney disease (PKD)) comprises administering about 60 mg of an anti-NPR1 antibody (e.g., N001_DAPA or N001_LALA) or antigen binding fragment thereof. In some embodiments, the methods of treating a disorder or a disease associated with natriuretic peptide receptor 1 (NPR1) activity (e.g., preeclampsia, asthma, glaucoma, cytokine release syndrome or a kidney disorder such as diabetic renal insufficiency, non-diabetic renal insufficiency, renal failure, diabetic nephropathy, non-diabetic nephropathy, acute renal injury, contrast induced nephropathy, nephrotic syndrome, glomerulonephritis, scleroderma, glomerular sclerosis, proteinuria of primary renal disease, renal vascular hypertension, diabetic retinopathy and end-stage renal disease (ESRD), endothelial dysfunction, diastolic dysfunction, renal fibrosis, and polycystic kidney disease (PKD)) comprises administering about 120 mg of an anti-NPR1 antibody (e.g., N001_DAPA or N001_LALA) or antigen binding fragment thereof. In some embodiments, the methods of treating a disorder or a disease associated with natriuretic peptide receptor 1 (NPR1) activity (e.g., preeclampsia, asthma, glaucoma, cytokine release syndrome or a kidney disorder such as diabetic renal insufficiency, non-diabetic renal insufficiency, renal failure, diabetic nephropathy, non-diabetic nephropathy, acute renal injury, contrast induced nephropathy, nephrotic syndrome, glomerulonephritis, scleroderma, glomerular sclerosis, proteinuria of primary renal disease, renal vascular hypertension, diabetic retinopathy and end-stage renal disease (ESRD), endothelial dysfunction, diastolic dysfunction, renal fibrosis, and polycystic kidney disease (PKD)) comprises administering about 240 mg of an anti-NPR1 antibody (e.g., N001_DAPA or N001_LALA) or antigen binding fragment thereof.

In some embodiments, provided herein are methods of treating hypertension, peripheral vascular disease, heart failure, coronary artery disease (CAD), ischemic heart disease (IHD), mitral stenosis and regurgitation, angina, hypertrophic cardiomyopathy, diabetic cardiomyopathy, supraventricular and ventricular arrhythmias, cardiac dysrhythmia, atrial fibrillation (AF), new onset of atrial fibrillation, recurrent atrial fibrillation, cardiac fibrosis, atrial flutter, detrimental vascular remodeling, plaque stabilization, or myocardial infarction (MI) in a subject in need thereof, the method comprising administering to the subject about 60 mg of an anti-NPR1 antibody (e.g., N001_DAPA or N001_LALA) or antigen binding fragment thereof. In some embodiments, provided herein are methods of treating hypertension, peripheral vascular disease, heart failure, coronary artery disease (CAD), ischemic heart disease (IHD), mitral stenosis and regurgitation, angina, hypertrophic cardiomyopathy, diabetic cardiomyopathy, supraventricular and ventricular arrhythmias, cardiac dysrhythmia, atrial fibrillation (AF), new onset of atrial fibrillation, recurrent atrial fibrillation, cardiac fibrosis, atrial flutter, detrimental vascular remodeling, plaque stabilization, or myocardial infarction (MI) in a subject in need thereof, the method comprising administering to the subject about 70 mg of an anti-NPR1 antibody (e.g., N001_DAPA or N001_LALA) or antigen binding fragment thereof. In some embodiments, provided herein are methods of treating hypertension, peripheral vascular disease, heart failure, coronary artery disease (CAD), ischemic heart disease (IHD), mitral stenosis and regurgitation, angina, hypertrophic cardiomyopathy, diabetic cardiomyopathy, supraventricular and ventricular arrhythmias, cardiac dysrhythmia, atrial fibrillation (AF), new onset of atrial fibrillation, recurrent atrial fibrillation, cardiac fibrosis, atrial flutter, detrimental vascular remodeling, plaque stabilization, or myocardial infarction (MI) in a subject in need thereof, the method comprising administering to the subject about 80 mg of an anti-NPR1 antibody (e.g., N001_DAPA or N001_LALA) or antigen binding fragment thereof. In some embodiments, provided herein are methods of treating hypertension, peripheral vascular disease, heart failure, coronary artery disease (CAD), ischemic heart disease (IHD), mitral stenosis and regurgitation, angina, hypertrophic cardiomyopathy, diabetic cardiomyopathy, supraventricular and ventricular arrhythmias, cardiac dysrhythmia, atrial fibrillation (AF), new onset of atrial fibrillation, recurrent atrial fibrillation, cardiac fibrosis, atrial flutter, detrimental vascular remodeling, plaque stabilization, or myocardial infarction (MI) in a subject in need thereof, the method comprising administering to the subject about 90 mg of an anti-NPR1 antibody (e.g., N001_DAPA or N001_LALA) or antigen binding fragment thereof. In some embodiments, provided herein are methods of treating hypertension, peripheral vascular disease, heart failure, coronary artery disease (CAD), ischemic heart disease (IHD), mitral stenosis and regurgitation, angina, hypertrophic cardiomyopathy, diabetic cardiomyopathy, supraventricular and ventricular arrhythmias, cardiac dysrhythmia, atrial fibrillation (AF), new onset of atrial fibrillation, recurrent atrial fibrillation, cardiac fibrosis, atrial flutter, detrimental vascular remodeling, plaque stabilization, or myocardial infarction (MI) in a subject in need thereof, the method comprising administering to the subject about 100 mg of an anti-NPR1 antibody (e.g., N001_DAPA or N001_LALA) or antigen binding fragment thereof.

In some embodiments, provided herein are methods of treating hypertension, peripheral vascular disease, heart failure, coronary artery disease (CAD), ischemic heart disease (IHD), mitral stenosis and regurgitation, angina, hypertrophic cardiomyopathy, diabetic cardiomyopathy, supraventricular and ventricular arrhythmias, cardiac dysrhythmia, atrial fibrillation (AF), new onset of atrial fibrillation, recurrent atrial fibrillation, cardiac fibrosis, atrial flutter, detrimental vascular remodeling, plaque stabilization, or myocardial infarction (MI) in a subject in need thereof, the method comprising administering to the subject about 110 mg of an anti-NPR1 antibody (e.g., N001_DAPA or N001_LALA) or antigen binding fragment thereof. In some embodiments, provided herein are methods of treating hypertension, peripheral vascular disease, heart failure, coronary artery disease (CAD), ischemic heart disease (IHD), mitral stenosis and regurgitation, angina, hypertrophic cardiomyopathy, diabetic cardiomyopathy, supraventricular and ventricular arrhythmias, cardiac dysrhythmia, atrial fibrillation (AF), new onset of atrial fibrillation, recurrent atrial fibrillation, cardiac fibrosis, atrial flutter, detrimental vascular remodeling, plaque stabilization, or myocardial infarction (MI) in a subject in need thereof, the method comprising administering to the subject about 120 mg of an anti-NPR1 antibody (e.g., N001_DAPA or N001_LALA) or antigen binding fragment thereof. In some embodiments, provided herein are methods of treating hypertension, peripheral vascular disease, heart failure, coronary artery disease (CAD), ischemic heart disease (IHD), mitral stenosis and regurgitation, angina, hypertrophic cardiomyopathy, diabetic cardiomyopathy, supraventricular and ventricular arrhythmias, cardiac dysrhythmia, atrial fibrillation (AF), new onset of atrial fibrillation, recurrent atrial fibrillation, cardiac fibrosis, atrial flutter, detrimental vascular remodeling, plaque stabilization, or myocardial infarction (MI) in a subject in need thereof, the method comprising administering to the subject about 130 mg of an anti-NPR1 antibody (e.g., N001_DAPA or N001_LALA) or antigen binding fragment thereof. In some embodiments, provided herein are methods of treating hypertension, peripheral vascular disease, heart failure, coronary artery disease (CAD), ischemic heart disease (IHD), mitral stenosis and regurgitation, angina, hypertrophic cardiomyopathy, diabetic cardiomyopathy, supraventricular and ventricular arrhythmias, cardiac dysrhythmia, atrial fibrillation (AF), new onset of atrial fibrillation, recurrent atrial fibrillation, cardiac fibrosis, atrial flutter, detrimental vascular remodeling, plaque stabilization, or myocardial infarction (MI) in a subject in need thereof, the method comprising administering to the subject about 140 mg of an anti-NPR1 antibody (e.g., N001_DAPA or N001_LALA) or antigen binding fragment thereof. In some embodiments, provided herein are methods of treating hypertension, peripheral vascular disease, heart failure, coronary artery disease (CAD), ischemic heart disease (IHD), mitral stenosis and regurgitation, angina, hypertrophic cardiomyopathy, diabetic cardiomyopathy, supraventricular and ventricular arrhythmias, cardiac dysrhythmia, atrial fibrillation (AF), new onset of atrial fibrillation, recurrent atrial fibrillation, cardiac fibrosis, atrial flutter, detrimental vascular remodeling, plaque stabilization, or myocardial infarction (MI) in a subject in need thereof, the method comprising administering to the subject about 150 mg of an anti-NPR1 antibody (e.g., N001_DAPA or N001_LALA) or antigen binding fragment thereof. In some embodiments, provided herein are methods of treating hypertension, peripheral vascular disease, heart failure, coronary artery disease (CAD), ischemic heart disease (IHD), mitral stenosis and regurgitation, angina, hypertrophic cardiomyopathy, diabetic cardiomyopathy, supraventricular and ventricular arrhythmias, cardiac dysrhythmia, atrial fibrillation (AF), new onset of atrial fibrillation, recurrent atrial fibrillation, cardiac fibrosis, atrial flutter, detrimental vascular remodeling, plaque stabilization, or myocardial infarction (MI) in a subject in need thereof, the method comprising administering to the subject about 160 mg of an anti-NPR1 antibody (e.g., N001_DAPA or N001_LALA) or antigen binding fragment thereof. In some embodiments, provided herein are methods of treating hypertension, peripheral vascular disease, heart failure, coronary artery disease (CAD), ischemic heart disease (IHD), mitral stenosis and regurgitation, angina, hypertrophic cardiomyopathy, diabetic cardiomyopathy, supraventricular and ventricular arrhythmias, cardiac dysrhythmia, atrial fibrillation (AF), new onset of atrial fibrillation, recurrent atrial fibrillation, cardiac fibrosis, atrial flutter, detrimental vascular remodeling, plaque stabilization, or myocardial infarction (MI) in a subject in need thereof, the method comprising administering to the subject about 170 mg of an anti-NPR1 antibody (e.g., N001_DAPA or N001_LALA) or antigen binding fragment thereof. In some embodiments, provided herein are methods of treating hypertension, peripheral vascular disease, heart failure, coronary artery disease (CAD), ischemic heart disease (IHD), mitral stenosis and regurgitation, angina, hypertrophic cardiomyopathy, diabetic cardiomyopathy, supraventricular and ventricular arrhythmias, cardiac dysrhythmia, atrial fibrillation (AF), new onset of atrial fibrillation, recurrent atrial fibrillation, cardiac fibrosis, atrial flutter, detrimental vascular remodeling, plaque stabilization, or myocardial infarction (MI) in a subject in need thereof, the method comprising administering to the subject about 180 mg of an anti-NPR1 antibody (e.g., N001_DAPA or N001_LALA) or antigen binding fragment thereof. In some embodiments, provided herein are methods of treating hypertension, peripheral vascular disease, heart failure, coronary artery disease (CAD), ischemic heart disease (IHD), mitral stenosis and regurgitation, angina, hypertrophic cardiomyopathy, diabetic cardiomyopathy, supraventricular and ventricular arrhythmias, cardiac dysrhythmia, atrial fibrillation (AF), new onset of atrial fibrillation, recurrent atrial fibrillation, cardiac fibrosis, atrial flutter, detrimental vascular remodeling, plaque stabilization, or myocardial infarction (MI) in a subject in need thereof, the method comprising administering to the subject about 190 mg of an anti-NPR1 antibody (e.g., N001_DAPA or N001_LALA) or antigen binding fragment thereof. In some embodiments, provided herein are methods of treating hypertension, peripheral vascular disease, heart failure, coronary artery disease (CAD), ischemic heart disease (IHD), mitral stenosis and regurgitation, angina, hypertrophic cardiomyopathy, diabetic cardiomyopathy, supraventricular and ventricular arrhythmias, cardiac dysrhythmia, atrial fibrillation (AF), new onset of atrial fibrillation, recurrent atrial fibrillation, cardiac fibrosis, atrial flutter, detrimental vascular remodeling, plaque stabilization, or myocardial infarction (MI) in a subject in need thereof, the method comprising administering to the subject about 200 mg of an anti-NPR1 antibody (e.g., N001_DAPA or N001_LALA) or antigen binding fragment thereof.

In some embodiments, provided herein are methods of treating hypertension, peripheral vascular disease, heart failure, coronary artery disease (CAD), ischemic heart disease (IHD), mitral stenosis and regurgitation, angina, hypertrophic cardiomyopathy, diabetic cardiomyopathy, supraventricular and ventricular arrhythmias, cardiac dysrhythmia, atrial fibrillation (AF), new onset of atrial fibrillation, recurrent atrial fibrillation, cardiac fibrosis, atrial flutter, detrimental vascular remodeling, plaque stabilization, or myocardial infarction (MI) in a subject in need thereof, the method comprising administering to the subject about 210 mg of an anti-NPR1 antibody (e.g., N001_DAPA or N001_LALA) or antigen binding fragment thereof. In some embodiments, provided herein are methods of treating hypertension, peripheral vascular disease, heart failure, coronary artery disease (CAD), ischemic heart disease (IHD), mitral stenosis and regurgitation, angina, hypertrophic cardiomyopathy, diabetic cardiomyopathy, supraventricular and ventricular arrhythmias, cardiac dysrhythmia, atrial fibrillation (AF), new onset of atrial fibrillation, recurrent atrial fibrillation, cardiac fibrosis, atrial flutter, detrimental vascular remodeling, plaque stabilization, or myocardial infarction (MI) in a subject in need thereof, the method comprising administering to the subject about 220 mg of an anti-NPR1 antibody (e.g., N001_DAPA or N001_LALA) or antigen binding fragment thereof. In some embodiments, provided herein are methods of treating hypertension, peripheral vascular disease, heart failure, coronary artery disease (CAD), ischemic heart disease (IHD), mitral stenosis and regurgitation, angina, hypertrophic cardiomyopathy, diabetic cardiomyopathy, supraventricular and ventricular arrhythmias, cardiac dysrhythmia, atrial fibrillation (AF), new onset of atrial fibrillation, recurrent atrial fibrillation, cardiac fibrosis, atrial flutter, detrimental vascular remodeling, plaque stabilization, or myocardial infarction (MI) in a subject in need thereof, the method comprising administering to the subject about 230 mg of an anti-NPR1 antibody (e.g., N001_DAPA or N001_LALA) or antigen binding fragment thereof. In some embodiments, provided herein are methods of treating hypertension, peripheral vascular disease, heart failure, coronary artery disease (CAD), ischemic heart disease (IHD), mitral stenosis and regurgitation, angina, hypertrophic cardiomyopathy, diabetic cardiomyopathy, supraventricular and ventricular arrhythmias, cardiac dysrhythmia, atrial fibrillation (AF), new onset of atrial fibrillation, recurrent atrial fibrillation, cardiac fibrosis, atrial flutter, detrimental vascular remodeling, plaque stabilization, or myocardial infarction (MI) in a subject in need thereof, the method comprising administering to the subject about 240 mg of an anti-NPR1 antibody (e.g., N001_DAPA or N001_LALA) or antigen binding fragment thereof. In some embodiments, provided herein are methods of treating hypertension, peripheral vascular disease, heart failure, coronary artery disease (CAD), ischemic heart disease (IHD), mitral stenosis and regurgitation, angina, hypertrophic cardiomyopathy, diabetic cardiomyopathy, supraventricular and ventricular arrhythmias, cardiac dysrhythmia, atrial fibrillation (AF), new onset of atrial fibrillation, recurrent atrial fibrillation, cardiac fibrosis, atrial flutter, detrimental vascular remodeling, plaque stabilization, or myocardial infarction (MI) in a subject in need thereof, the method comprising administering to the subject about 300 mg of an anti-NPR1 antibody (e.g., N001_DAPA or N001_LALA) or antigen binding fragment thereof. In some embodiments, provided herein are methods of treating hypertension, peripheral vascular disease, heart failure, coronary artery disease (CAD), ischemic heart disease (IHD), mitral stenosis and regurgitation, angina, hypertrophic cardiomyopathy, diabetic cardiomyopathy, supraventricular and ventricular arrhythmias, cardiac dysrhythmia, atrial fibrillation (AF), new onset of atrial fibrillation, recurrent atrial fibrillation, cardiac fibrosis, atrial flutter, detrimental vascular remodeling, plaque stabilization, or myocardial infarction (MI) in a subject in need thereof, the method comprising administering to the subject about 350 mg of an anti-NPR1 antibody (e.g., N001_DAPA or N001_LALA) or antigen binding fragment thereof. In some embodiments, provided herein are methods of treating hypertension, peripheral vascular disease, heart failure, coronary artery disease (CAD), ischemic heart disease (IHD), mitral stenosis and regurgitation, angina, hypertrophic cardiomyopathy, diabetic cardiomyopathy, supraventricular and ventricular arrhythmias, cardiac dysrhythmia, atrial fibrillation (AF), new onset of atrial fibrillation, recurrent atrial fibrillation, cardiac fibrosis, atrial flutter, detrimental vascular remodeling, plaque stabilization, or myocardial infarction (MI) in a subject in need thereof, the method comprising administering to the subject about 400 mg of an anti-NPR1 antibody (e.g., N001_DAPA or N001_LALA) or antigen binding fragment thereof. In some embodiments, provided herein are methods of treating hypertension, peripheral vascular disease, heart failure, coronary artery disease (CAD), ischemic heart disease (IHD), mitral stenosis and regurgitation, angina, hypertrophic cardiomyopathy, diabetic cardiomyopathy, supraventricular and ventricular arrhythmias, cardiac dysrhythmia, atrial fibrillation (AF), new onset of atrial fibrillation, recurrent atrial fibrillation, cardiac fibrosis, atrial flutter, detrimental vascular remodeling, plaque stabilization, or myocardial infarction (MI) in a subject in need thereof, the method comprising administering to the subject about 450 mg of an anti-NPR1 antibody (e.g., N001_DAPA or N001_LALA) or antigen binding fragment thereof. In some embodiments, provided herein are methods of treating hypertension, peripheral vascular disease, heart failure, coronary artery disease (CAD), ischemic heart disease (IHD), mitral stenosis and regurgitation, angina, hypertrophic cardiomyopathy, diabetic cardiomyopathy, supraventricular and ventricular arrhythmias, cardiac dysrhythmia, atrial fibrillation (AF), new onset of atrial fibrillation, recurrent atrial fibrillation, cardiac fibrosis, atrial flutter, detrimental vascular remodeling, plaque stabilization, or myocardial infarction (MI) in a subject in need thereof, the method comprising administering to the subject about 500 mg of an anti-NPR1 antibody (e.g., N001_DAPA or N001_LALA) or antigen binding fragment thereof. In some embodiments, provided herein are methods of treating hypertension, peripheral vascular disease, heart failure, coronary artery disease (CAD), ischemic heart disease (IHD), mitral stenosis and regurgitation, angina, hypertrophic cardiomyopathy, diabetic cardiomyopathy, supraventricular and ventricular arrhythmias, cardiac dysrhythmia, atrial fibrillation (AF), new onset of atrial fibrillation, recurrent atrial fibrillation, cardiac fibrosis, atrial flutter, detrimental vascular remodeling, plaque stabilization, or myocardial infarction (MI) in a subject in need thereof, the method comprising administering to the subject about 550 mg of an anti-NPR1 antibody (e.g., N001_DAPA or N001_LALA) or antigen binding fragment thereof. In some embodiments, provided herein are methods of treating hypertension, peripheral vascular disease, heart failure, coronary artery disease (CAD), ischemic heart disease (IHD), mitral stenosis and regurgitation, angina, hypertrophic cardiomyopathy, diabetic cardiomyopathy, supraventricular and ventricular arrhythmias, cardiac dysrhythmia, atrial fibrillation (AF), new onset of atrial fibrillation, recurrent atrial fibrillation, cardiac fibrosis, atrial flutter, detrimental vascular remodeling, plaque stabilization, or myocardial infarction (MI) in a subject in need thereof, the method comprising administering to the subject about 600 mg of an anti-NPR1 antibody (e.g., N001_DAPA or N001_LALA) or antigen binding fragment thereof. In some embodiments, provided herein are methods of treating hypertension, peripheral vascular disease, heart failure, coronary artery disease (CAD), ischemic heart disease (IHD), mitral stenosis and regurgitation, angina, hypertrophic cardiomyopathy, diabetic cardiomyopathy, supraventricular and ventricular arrhythmias, cardiac dysrhythmia, atrial fibrillation (AF), new onset of atrial fibrillation, recurrent atrial fibrillation, cardiac fibrosis, atrial flutter, detrimental vascular remodeling, plaque stabilization, or myocardial infarction (MI) in a subject in need thereof, the method comprising administering to the subject about 650 mg of an anti-NPR1 antibody (e.g., N001_DAPA or N001_LALA) or antigen binding fragment thereof. In some embodiments, provided herein are methods of treating hypertension, peripheral vascular disease, heart failure, coronary artery disease (CAD), ischemic heart disease (IHD), mitral stenosis and regurgitation, angina, hypertrophic cardiomyopathy, diabetic cardiomyopathy, supraventricular and ventricular arrhythmias, cardiac dysrhythmia, atrial fibrillation (AF), new onset of atrial fibrillation, recurrent atrial fibrillation, cardiac fibrosis, atrial flutter, detrimental vascular remodeling, plaque stabilization, or myocardial infarction (MI) in a subject in need thereof, the method comprising administering to the subject about 700 mg of an anti-NPR1 antibody (e.g., N001_DAPA or N001_LALA) or antigen binding fragment thereof.

In some embodiments, provided herein are methods of treating heart failure in a subject in need thereof, the method comprising administering to the subject about 10 mg, about 15 mg, about 20 mg, about 25 mg, about 30 mg, about 35 mg, about 40 mg, about 45 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 350 mg, about 400 mg, about 450 mg, about 500 mg, about 550 mg, about 600 mg, about 650 mg, or about 700 mg of an anti-NPR1 antibody (e.g., N001_DAPA or N001_LALA) or antigen binding fragment thereof. In some embodiments, provided herein are methods of treating heart failure in a subject in need thereof, the method comprising administering to the subject about 60 mg of an anti-NPR1 antibody (e.g., N001_DAPA or N001_LALA) or antigen binding fragment thereof. In some embodiments, provided herein are methods of treating heart failure in a subject in need thereof, the method comprising administering to the subject about 120 mg of an anti-NPR1 antibody (e.g., N001_DAPA or N001_LALA) or antigen binding fragment thereof. In some embodiments, provided herein are methods of treating heart failure in a subject in need thereof, the method comprising administering to the subject about 240 mg of an anti-NPR1 antibody (e.g., N001_DAPA or N001_LALA) or antigen binding fragment thereof. In some embodiments, provided herein are methods of treating heart failure with reduced ejection fraction (HFrEF) in a subject in need thereof, the method comprising administering to the subject about 10 mg, about 15 mg, about 20 mg, about 25 mg, about 30 mg, about 35 mg, about 40 mg, about 45 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 350 mg, about 400 mg, about 450 mg, about 500 mg, about 550 mg, about 600 mg, about 650 mg, or about 700 mg of an anti-NPR1 antibody (e.g., N001_DAPA or N001_LALA) or antigen binding fragment thereof. In some embodiments, provided herein are methods of treating heart failure with reduced ejection fraction (HFrEF) in a subject in need thereof, the method comprising administering to the subject about 60 mg of an anti-NPR1 antibody (e.g., N001_DAPA or N001_LALA) or antigen binding fragment thereof. In some embodiments, provided herein are methods of treating heart failure with reduced ejection fraction (HFrEF) in a subject in need thereof, the method comprising administering to the subject about 120 mg of an anti-NPR1 antibody (e.g., N001_DAPA or N001_LALA) or antigen binding fragment thereof. In some embodiments, provided herein are methods of treating heart failure with reduced ejection fraction (HFrEF) in a subject in need thereof, the method comprising administering to the subject about 240 mg of an anti-NPR1 antibody (e.g., N001_DAPA or N001_LALA) or antigen binding fragment thereof. In some embodiments, provided herein are methods of treating heart failure with mid-range ejection fraction or heart failure with mildly reduced ejection fraction or heart failure with mildly reduced ejection fraction (HFmrEF) in a subject in need thereof, the method comprising administering to the subject about 10 mg, about 15 mg, about 20 mg, about 25 mg, about 30 mg, about 35 mg, about 40 mg, about 45 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 350 mg, about 400 mg, about 450 mg, about 500 mg, about 550 mg, about 600 mg, about 650 mg, or about 700 mg of an anti-NPR1 antibody (e.g., N001_DAPA or N001_LALA) or antigen binding fragment thereof. In some embodiments, provided herein are methods of treating heart failure with mid-range ejection fraction or heart failure with mildly reduced ejection fraction or heart failure with mildly reduced ejection fraction (HFmrEF) in a subject in need thereof, the method comprising administering to the subject about 60 mg of an anti-NPR1 antibody (e.g., N001_DAPA or N001_LALA) or antigen binding fragment thereof. In some embodiments, provided herein are methods of treating heart failure with mid-range ejection fraction or heart failure with mildly reduced ejection fraction (HFmrEF) in a subject in need thereof, the method comprising administering to the subject about 120 mg of an anti-NPR1 antibody (e.g., N001_DAPA or N001_LALA) or antigen binding fragment thereof. In some embodiments, provided herein are methods of treating heart failure with mid-range ejection fraction or heart failure with mildly reduced ejection fraction (HFmrEF) in a subject in need thereof, the method comprising administering to the subject about 240 mg of an anti-NPR1 antibody (e.g., N001_DAPA or N001_LALA) or antigen binding fragment thereof. In some embodiments, provided herein are methods of treating heart failure with preserved ejection fraction (HFpEF) in a subject in need thereof, the method comprising administering to the subject about 10 mg, about 15 mg, about 20 mg, about 25 mg, about 30 mg, about 35 mg, about 40 mg, about 45 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 350 mg, about 400 mg, about 450 mg, about 500 mg, about 550 mg, about 600 mg, about 650 mg, or about 700 mg of an anti-NPR1 antibody (e.g., N001_DAPA or N001_LALA) or antigen binding fragment thereof. In some embodiments, provided herein are methods of treating heart failure after acute myocardial infarct in a subject in need thereof, the method comprising administering to the subject about 10 mg, about 15 mg, about 20 mg, about 25 mg, about 30 mg, about 35 mg, about 40 mg, about 45 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 350 mg, about 400 mg, about 450 mg, about 500 mg, about 550 mg, about 600 mg, about 650 mg, or about 700 mg of an anti-NPR1 antibody (e.g., N001_DAPA or N001_LALA) or antigen binding fragment thereof. In some embodiments, provided herein are methods of treating acute decompensated heart failure in a subject in need thereof, the method comprising administering to the subject about 10 mg, about 15 mg, about 20 mg, about 25 mg, about 30 mg, about 35 mg, about 40 mg, about 45 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 350 mg, about 400 mg, about 450 mg, about 500 mg, about 550 mg, about 600 mg, about 650 mg, or about 700 mg of an anti-NPR1 antibody (e.g., N001_DAPA or N001_LALA) or antigen binding fragment thereof.

In some embodiments, provided herein are methods of treating hypertension in a subject in need thereof, the method comprising administering to the subject about 10 mg, about 15 mg, about 20 mg, about 25 mg, about 30 mg, about 35 mg, about 40 mg, about 45 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 350 mg, about 400 mg, about 450 mg, about 500 mg, about 550 mg, about 600 mg, about 650 mg, or about 700 mg of an anti-NPR1 antibody (e.g., N001_DAPA or N001_LALA) or antigen binding fragment thereof. In some embodiments, provided herein are methods of treating hypertension in a subject in need thereof, the method comprising administering to the subject about 60 mg of an anti-NPR1 antibody (e.g., N001_DAPA or N001_LALA) or antigen binding fragment thereof. In some embodiments, provided herein are methods of treating hypertension in a subject in need thereof, the method comprising administering to the subject about 120 mg of an anti-NPR1 antibody (e.g., N001_DAPA or N001_LALA) or antigen binding fragment thereof. In some embodiments, provided herein are methods of treating hypertension in a subject in need thereof, the method comprising administering to the subject about 240 mg of an anti-NPR1 antibody (e.g., N001_DAPA or N001_LALA) or antigen binding fragment thereof. In some embodiments, provided herein are methods of treating resistant hypertension in a subject in need thereof, the method comprising administering to the subject about 10 mg, about 15 mg, about 20 mg, about 25 mg, about 30 mg, about 35 mg, about 40 mg, about 45 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 350 mg, about 400 mg, about 450 mg, about 500 mg, about 550 mg, about 600 mg, about 650 mg, or about 700 mg of an anti-NPR1 antibody (e.g., N001_DAPA or N001_LALA) or antigen binding fragment thereof. In some embodiments, provided herein are methods of treating resistant hypertension in a subject in need thereof, the method comprising administering to the subject about 60 mg of an anti-NPR1 antibody (e.g., N001_DAPA or N001_LALA) or antigen binding fragment thereof. In some embodiments, provided herein are methods of treating resistant hypertension in a subject in need thereof, the method comprising administering to the subject about 120 mg of an anti-NPR1 antibody (e.g., N001_DAPA or N001_LALA) or antigen binding fragment thereof. In some embodiments, provided herein are methods of treating resistant hypertension in a subject in need thereof, the method comprising administering to the subject about 240 mg of an anti-NPR1 antibody (e.g., N001_DAPA or N001_LALA) or antigen binding fragment thereof. In some embodiments, provided herein are methods of treating hypertensive heart disease in a subject in need thereof, the method comprising administering to the subject about 10 mg, about 15 mg, about 20 mg, about 25 mg, about 30 mg, about 35 mg, about 40 mg, about 45 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 350 mg, about 400 mg, about 450 mg, about 500 mg, about 550 mg, about 600 mg, about 650 mg, or about 700 mg of an anti-NPR1 antibody (e.g., N001_DAPA or N001_LALA) or antigen binding fragment thereof. In some embodiments, provided herein are methods of treating pulmonary hypertension in a subject in need thereof, the method comprising administering to the subject about 10 mg, about 15 mg, about 20 mg, about 25 mg, about 30 mg, about 35 mg, about 40 mg, about 45 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 350 mg, about 400 mg, about 450 mg, about 500 mg, about 550 mg, about 600 mg, about 650 mg, or about 700 mg of an anti-NPR1 antibody (e.g., N001_DAPA or N001_LALA) or antigen binding fragment thereof. In some embodiments, provided herein are methods of treating pulmonary arterial hypertension in a subject in need thereof, the method comprising administering to the subject about 10 mg, about 15 mg, about 20 mg, about 25 mg, about 30 mg, about 35 mg, about 40 mg, about 45 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 350 mg, about 400 mg, about 450 mg, about 500 mg, about 550 mg, about 600 mg, about 650 mg, or about 700 mg of an anti-NPR1 antibody (e.g., N001_DAPA or N001_LALA) or antigen binding fragment thereof. In some embodiments, provided herein are methods of treating isolated systolic hypertension in a subject in need thereof, the method comprising administering to the subject about 10 mg, about 15 mg, about 20 mg, about 25 mg, about 30 mg, about 35 mg, about 40 mg, about 45 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 350 mg, about 400 mg, about 450 mg, about 500 mg, about 550 mg, about 600 mg, about 650 mg, or about 700 mg of an anti-NPR1 antibody (e.g., N001_DAPA or N001_LALA) or antigen binding fragment thereof.

In some embodiments, any of the methods or uses described herein comprise administering the anti-NPR1 antibody (e.g., N001_DAPA or N001_LALA) or antigen binding fragment thereof about once every week, once every 2 weeks, about once every 3 weeks, about once every 4 weeks, about once every 5 weeks, about once every 6 weeks, about once every 7 weeks, about once every 8 weeks, about once every 9 weeks, about once every 10 weeks, about once every 11 weeks, or about once every 12 weeks.

In some embodiments, provided herein are methods of treating a disorder or a disease associated with natriuretic peptide receptor 1 (NPR1) activity (e.g., a cardiovascular disorder) in a subject in need thereof, the method comprising administering to the subject about 10 mg, about 15 mg, about 20 mg, about 25 mg, about 30 mg, about 35 mg, about 40 mg, about 45 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 350 mg, about 400 mg, about 450 mg, about 500 mg, about 550 mg, about 600 mg, about 650 mg, or about 700 mg of an anti-NPR1 antibody (e.g., N001_DAPA or N001_LALA) or antigen binding fragment thereof, wherein the anti-NPR1 antibody (e.g., N001_DAPA or N001_LALA) or antigen binding fragment thereof is administered about every week. In some embodiments, provided herein are methods of treating a disorder or a disease associated with natriuretic peptide receptor 1 (NPR1) activity (e.g., a cardiovascular disorder) in a subject in need thereof, the method comprising administering to the subject about 10 mg, about 15 mg, about 20 mg, about 25 mg, about 30 mg, about 35 mg, about 40 mg, about 45 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 350 mg, about 400 mg, about 450 mg, about 500 mg, about 550 mg, about 600 mg, about 650 mg, or about 700 mg of an anti-NPR1 antibody (e.g., N001_DAPA or N001_LALA) or antigen binding fragment thereof, wherein the anti-NPR1 antibody (e.g., N001_DAPA or N001_LALA) or antigen binding fragment thereof is administered about every two weeks. In some embodiments, provided herein are methods of treating a disorder or a disease associated with natriuretic peptide receptor 1 (NPR1) activity (e.g., a cardiovascular disorder) in a subject in need thereof, the method comprising administering to the subject about 10 mg, about 15 mg, about 20 mg, about 25 mg, about 30 mg, about 35 mg, about 40 mg, about 45 mg, about 10 mg, about 15 mg, about 20 mg, about 25 mg, about 30 mg, about 35 mg, about 40 mg, about 45 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 350 mg, about 400 mg, about 450 mg, about 500 mg, about 550 mg, about 600 mg, about 650 mg, or about 700 mg of an anti-NPR1 antibody (e.g., N001_DAPA or N001_LALA) or antigen binding fragment thereof, wherein the anti-NPR1 antibody (e.g., N001_DAPA or N001_LALA) or antigen binding fragment thereof is administered about every three weeks. In some embodiments, provided herein are methods of treating a disorder or a disease associated with natriuretic peptide receptor 1 (NPR1) activity (e.g., a cardiovascular disorder) in a subject in need thereof, the method comprising administering to the subject about 10 mg, about 15 mg, about 20 mg, about 25 mg, about 30 mg, about 35 mg, about 40 mg, about 45 mg, about 10 mg, about 15 mg, about 20 mg, about 25 mg, about 30 mg, about 35 mg, about 40 mg, about 45 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 350 mg, about 400 mg, about 450 mg, about 500 mg, about 550 mg, about 600 mg, about 650 mg, or about 700 mg of an anti-NPR1 antibody (e.g., N001_DAPA or N001_LALA) or antigen binding fragment thereof, wherein the anti-NPR1 antibody (e.g., N001_DAPA or N001_LALA) or antigen binding fragment thereof is administered about every four weeks. In some embodiments, provided herein are methods of treating a disorder or a disease associated with natriuretic peptide receptor 1 (NPR1) activity (e.g., a cardiovascular disorder) in a subject in need thereof, the method comprising administering to the subject about 10 mg, about 15 mg, about 20 mg, about 25 mg, about 30 mg, about 35 mg, about 40 mg, about 45 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 350 mg, about 400 mg, about 450 mg, about 500 mg, about 550 mg, about 600 mg, about 650 mg, or about 700 mg of an anti-NPR1 antibody (e.g., N001_DAPA or N001_LALA) or antigen binding fragment thereof, wherein the anti-NPR1 antibody (e.g., N001_DAPA or N001_LALA) or antigen binding fragment thereof is administered about every five weeks. In some embodiments, provided herein are methods of treating a disorder or a disease associated with natriuretic peptide receptor 1 (NPR1) activity (e.g., a cardiovascular disorder) in a subject in need thereof, the method comprising administering to the subject about 10 mg, about 15 mg, about 20 mg, about 25 mg, about 30 mg, about 35 mg, about 40 mg, about 45 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 350 mg, about 400 mg, about 450 mg, about 500 mg, about 550 mg, about 600 mg, about 650 mg, or about 700 mg of an anti-NPR1 antibody (e.g., N001_DAPA or N001_LALA) or antigen binding fragment thereof, wherein the anti-NPR1 antibody (e.g., N001_DAPA or N001_LALA) or antigen binding fragment thereof is administered about every six weeks. In some embodiments, provided herein are methods of treating a disorder or a disease associated with natriuretic peptide receptor 1 (NPR1) activity (e.g., a cardiovascular disorder) in a subject in need thereof, the method comprising administering to the subject about 10 mg, about 15 mg, about 20 mg, about 25 mg, about 30 mg, about 35 mg, about 40 mg, about 45 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 350 mg, about 400 mg, about 450 mg, about 500 mg, about 550 mg, about 600 mg, about 650 mg, or about 700 mg of an anti-NPR1 antibody (e.g., N001_DAPA or N001_LALA) or antigen binding fragment thereof, wherein the anti-NPR1 antibody (e.g., N001_DAPA or N001_LALA) or antigen binding fragment thereof is administered about every seven weeks. In some embodiments, provided herein are methods of treating a disorder or a disease associated with natriuretic peptide receptor 1 (NPR1) activity (e.g., a cardiovascular disorder) in a subject in need thereof, the method comprising administering to the subject about 10 mg, about 15 mg, about 20 mg, about 25 mg, about 30 mg, about 35 mg, about 40 mg, about 45 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 350 mg, about 400 mg, about 450 mg, about 500 mg, about 550 mg, about 600 mg, about 650 mg, or about 700 mg of an anti-NPR1 antibody (e.g., N001_DAPA or N001_LALA) or antigen binding fragment thereof, wherein the anti-NPR1 antibody (e.g., N001_DAPA or N001_LALA) or antigen binding fragment thereof is administered about every eight weeks. In some embodiments, provided herein are methods of treating a disorder or a disease associated with natriuretic peptide receptor 1 (NPR1) activity (e.g., a cardiovascular disorder) in a subject in need thereof, the method comprising administering to the subject about 10 mg, about 15 mg, about 20 mg, about 25 mg, about 30 mg, about 35 mg, about 40 mg, about 45 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 350 mg, about 400 mg, about 450 mg, about 500 mg, about 550 mg, about 600 mg, about 650 mg, or about 700 mg of an anti-NPR1 antibody (e.g., N001_DAPA or N001_LALA) or antigen binding fragment thereof, wherein the anti-NPR1 antibody (e.g., N001_DAPA or N001_LALA) or antigen binding fragment thereof is administered about every nine weeks. In some embodiments, provided herein are methods of treating a disorder or a disease associated with natriuretic peptide receptor 1 (NPR1) activity (e.g., a cardiovascular disorder) in a subject in need thereof, the method comprising administering to the subject about 10 mg, about 15 mg, about 20 mg, about 25 mg, about 30 mg, about 35 mg, about 40 mg, about 45 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 350 mg, about 400 mg, about 450 mg, about 500 mg, about 550 mg, about 600 mg, about 650 mg, or about 700 mg of an anti-NPR1 antibody (e.g., N001_DAPA or N001_LALA) or antigen binding fragment thereof, wherein the anti-NPR1 antibody (e.g., N001_DAPA or N001_LALA) or antigen binding fragment thereof is administered about every ten weeks. In some embodiments, provided herein are methods of treating a disorder or a disease associated with natriuretic peptide receptor 1 (NPR1) activity (e.g., a cardiovascular disorder) in a subject in need thereof, the method comprising administering to the subject about 10 mg, about 15 mg, about 20 mg, about 25 mg, about 30 mg, about 35 mg, about 40 mg, about 45 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 350 mg, about 400 mg, about 450 mg, about 500 mg, about 550 mg, about 600 mg, about 650 mg, or about 700 mg of an anti-NPR1 antibody (e.g., N001_DAPA or N001_LALA) or antigen binding fragment thereof, wherein the anti-NPR1 antibody (e.g., N001_DAPA or N001_LALA) or antigen binding fragment thereof is administered about every eleven weeks. In some embodiments, provided herein are methods of treating a disorder or a disease associated with natriuretic peptide receptor 1 (NPR1) activity (e.g., a cardiovascular disorder) in a subject in need thereof, the method comprising administering to the subject about 10 mg, about 15 mg, about 20 mg, about 25 mg, about 30 mg, about 35 mg, about 40 mg, about 45 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 350 mg, about 400 mg, about 450 mg, about 500 mg, about 550 mg, about 600 mg, about 650 mg, or about 700 mg of an anti-NPR1 antibody (e.g., N001_DAPA or N001_LALA) or antigen binding fragment thereof, wherein the anti-NPR1 antibody (e.g., N001_DAPA or N001_LALA) or antigen binding fragment thereof is administered about every twelve weeks.

In certain embodiments, provided herein are methods of treating a disorder or a disease associated with natriuretic peptide receptor 1 (NPR1) activity (e.g., a cardiovascular disorder) in a subject in need thereof, the method comprising administering to the subject different amounts of an anti-NPR1 antibody (e.g., N001_DAPA or N001_LALA) or antigen binding fragment thereof using an uptitration regimen. In such a regimen, a patient would receive an initial dose that is lower than a secondary or subsequent dose. In certain embodiments, the patient could also receive a tertiary dose that is higher than the secondary dose. In certain embodiments, the patient could also receive a quaternary dose that is higher than the tertiary dose. In certain embodiments, the patient could also receive one or more subsequent dose amounts (e.g., quinary, septenary, octonary, etc.) that are each higher (e.g., in mg) than the preceding dose amount. In certain embodiments, the increases in dose would reach a maximum dose level and would be maintained at that amount.

In certain embodiments, provided herein are methods of treating a disorder or a disease associated with natriuretic peptide receptor 1 (NPR1) activity (e.g., a cardiovascular disorder) in a subject in need thereof, the method comprising administering to the subject different amounts of an anti-NPR1 antibody (e.g., N001_DAPA or N001_LALA) or antigen binding fragment thereof. As a non-limiting set of examples, about 10 mg, about 15 mg, about 20 mg, about 25 mg, about 30 mg, about 35 mg, about 40 mg, about 45 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, or about 200 mg would be administered initially, and a secondary or subsequent administration would be at a higher amount, including, e.g., including about 15 mg, about 20 mg, about 25 mg, about 30 mg, about 35 mg, about 40 mg, about 45 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 350 mg, about 400 mg, about 450 mg, about 500 mg, about 550 mg, about 600 mg, about 650 mg, or about 700 mg of the anti-NPR1 antibody or antigen binding fragment thereof; as long as the secondary dose was greater than the initial dose.

In one embodiment, the initial, secondary, tertiary, quaternary, or further doses could be administered, for example, to the patient with a cardiovascular disorder every week for about one week, about two weeks, about three weeks, about four weeks, about five weeks, about six weeks, about seven weeks, or about eight weeks. In one embodiment, the initial, secondary, tertiary, quaternary, or further doses could be administered, for example, to the patient with a cardiovascular disorder every two weeks for about two weeks, about four weeks, about six weeks, about eight weeks, or about ten weeks. In one embodiment, the initial, secondary, tertiary, quaternary, or further doses could be administered, for example, to the patient with a cardiovascular disorder every two weeks for about two weeks, about four weeks, about six weeks, about eight weeks, or about ten weeks. In one embodiment, the initial, secondary, tertiary, quaternary, or further doses could be administered, for example, to the patient with a cardiovascular disorder every three weeks for about three weeks, about six weeks, about nine weeks, about twelve weeks, or about fifteen weeks. In one embodiment, the initial, secondary, tertiary, quaternary, or further doses could be administered, for example, to the patient with a cardiovascular disorder every month for about one month, about two months, about three months, about four months, about five months, about six months, about seven months, or about eight months.

In certain embodiments, the periods for each dosage amount might not be equal. For example, an initial dose (e.g., about 30 mg) could be administered at day 1; a secondary dose (e.g., about 60 mg) could be administered at week 2; and a tertiary dose (e.g., about 120 mg) could be administered once each month after that (i.e., at week 4, week 8, week 12, etc.).

As a non-limiting example, in certain embodiments a patient with a cardiovascular disorder could be administered an initial dose of about 30 mg, and could be administered the initial dose one or more (e.g., two, three, four, or five) times. Such a patient could then be administered a secondary dose of about 60 mg, and could be administered the secondary dose one or more (e.g., two, three, four, or five) times; or could be maintained on this dose for as long as necessary.

As a non-limiting example, in certain embodiments a patient with a cardiovascular disorder could be administered an initial dose of about 30 mg, and could be administered the initial dose one or more (e.g., two, three, four, or five) times. Such a patient could then be administered a secondary dose of about 60 mg, and could be administered the secondary dose one or more (e.g., two, three, four, or five) times. Such a patient could then be administered a tertiary dose of about 120 mg, and could be administered the tertiary dose one or more (e.g., two, three, four, or five) times. Such a patient could then be administered a quaternary dose of about 240 mg, and could be administered the quaternary dose one or more (e.g., two, three, four, or five) times; or could be maintained on this dose for as long as necessary.

As a non-limiting example, in certain embodiments a patient with a cardiovascular disorder could be administered an initial dose of about 30 mg, and could be administered the initial dose one or more (e.g., two, three, four, or five) times. Such a patient could then be administered a secondary dose of about 60 mg, and could be administered the secondary dose one or more (e.g., two, three, four, or five) times. Such a patient could then be administered a tertiary dose of about 120 mg, and could be administered the tertiary dose one or more (e.g., two, three, four, or five) times; or could be maintained on this dose for as long as necessary.

As a non-limiting example, in certain embodiments a patient with a cardiovascular disorder could be administered an initial dose of about 60 mg, and could be administered the initial dose one or more (e.g., two, three, four, or five) times. Such a patient could then be administered a secondary dose of about 120 mg, and could be administered the secondary dose one or more (e.g., two, three, four, or five) times; or could be maintained on this dose for as long as necessary.

As a non-limiting example, in certain embodiments a patient with a cardiovascular disorder could be administered an initial dose of about 60 mg, and could be administered the initial dose one or more (e.g., two, three, four, or five) times. Such a patient could then be administered a secondary dose of about 120 mg, and could be administered the secondary dose one or more (e.g., two, three, four, or five) times. Such a patient could then be administered a tertiary dose of about 240 mg, and could be administered the tertiary dose one or more (e.g., two, three, four, or five) times; or could be maintained on this dose for as long as necessary.

As a non-limiting example, in certain embodiments a patient with a cardiovascular disorder could be administered an initial dose of about 120 mg, and could be administered the initial dose one or more (e.g., two, three, four, or five) times. Such a patient could then be administered a secondary dose of about 240 mg, and could be administered the secondary dose one or more (e.g., two, three, four, or five) times; or could be maintained on this dose for as long as necessary.

As a non-limiting example, in certain embodiments a patient with a cardiovascular disorder could be administered an initial dose of about 60 mg, and could be administered the initial dose one or more (e.g., two, three, four, or five) times. Such a patient could then be administered a secondary dose of about 240 mg, and could be administered the secondary dose one or more (e.g., two, three, four, or five) times; or could be maintained on this dose for as long as necessary.

In certain embodiments the anti-NPR1 antibody could be administered to a patient with a cardiovascular disorder at an initial dose of about 30 mg at day 1 and week 2; and a secondary dose of about 60 mg every four weeks thereafter (i.e., at week 4, week 8, week 12, etc.).

In certain embodiments the anti-NPR1 antibody could be administered to a patient with a cardiovascular disorder at an initial dose of about 60 mg at day 1 and week 2; and a secondary dose of about 120 mg every four weeks thereafter (i.e., at week 4, week 8, week 12, etc.).

In certain embodiments the anti-NPR1 antibody could be administered to a patient with a cardiovascular disorder at an initial dose of about 30 mg at day 1; a secondary dose of about 60 mg at week 2; and a tertiary dose of about 120 mg every four weeks thereafter (i.e., at week 4, week 8, week 12, etc.).

In certain embodiments the anti-NPR1 antibody could be administered to a patient with a cardiovascular disorder at an initial dose of about 60 mg at day 1; and a secondary dose of about 120 mg every four weeks thereafter (i.e., at week 4, week 8, week 12, etc.).

In certain embodiments the anti-NPR1 antibody could be administered to a patient with a cardiovascular disorder at an initial dose of about 120 mg at day 1; and a secondary dose of about 240 mg every four weeks thereafter (i.e., at week 4, week 8, week 12, etc.).

In certain embodiments the anti-NPR1 antibody could be administered to a patient with a cardiovascular disorder at an initial dose of about 30 mg at day 1; a secondary dose of about 60 mg at week 2; a tertiary dose of about 120 mg at week 4; and a quaternary dose of about 240 mg every four weeks thereafter (i.e., at week 8, week 12, etc.).

In certain embodiments, provided herein are methods of treating a disorder or a disease associated with natriuretic peptide receptor 1 (NPR1) activity (e.g., a cardiovascular disorder) in a subject in need thereof, the method comprising administering to the subject different amounts of an anti-NPR1 antibody (e.g., N001_DAPA or N001_LALA) or antigen binding fragment thereof. As a non-limiting set of examples, about 10 mg, about 15 mg, about 20 mg, about 25 mg, about 30 mg, about 35 mg, about 40 mg, about 45 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, or about 200 mg could be administered initially, and a secondary or subsequent administration would be at a higher amount, including, e.g., including about 210 mg, about 220 mg, about 230 mg, about 240 mg, about 250 mg, about 260 mg, about 270 mg, about 280 mg, about 290 mg, about 300 mg, about 350 mg, about 400 mg, about 450 mg, about 500 mg, about 550 mg, about 600 mg, about 650 mg, or about 700 mg of the anti-NPR1 antibody or antigen binding fragment thereof. In one embodiment, about 120 mg of the anti-NPR1 antibody is administered in one or more initial doses (e.g., a first or second dose), and about 240 mg of the anti-NPR 1 antibody or antigen binding fragment thereof is administered in a secondary or subsequent dose (e.g., a second dose, a third dose, or further doses).

In some embodiments, provided herein are methods of treating heart failure (e.g., HFrEF) in a subject in need thereof, the method comprising administering to the subject about 10 mg, about 15 mg, about 20 mg, about 25 mg, about 30 mg, about 35 mg, about 40 mg, about 45 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 350 mg, about 400 mg, about 450 mg, about 500 mg, about 550 mg, about 600 mg, about 650 mg, or about 700 mg of an anti-NPR1 antibody (e.g., N001_DAPA or N001_LALA) or antigen binding fragment thereof, wherein the anti-NPR1 antibody (e.g., N001_DAPA or N001_LALA) or antigen binding fragment thereof is administered about every week. In some embodiments, provided herein are methods of treating heart failure (e.g., HFrEF) in a subject in need thereof, the method comprising administering to the subject about 10 mg, about 15 mg, about 20 mg, about 25 mg, about 30 mg, about 35 mg, about 40 mg, about 45 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 350 mg, about 400 mg, about 450 mg, about 500 mg, about 550 mg, about 600 mg, about 650 mg, or about 700 mg of an anti-NPR1 antibody (e.g., N001_DAPA or N001_LALA) or antigen binding fragment thereof, wherein the anti-NPR1 antibody (e.g., N001_DAPA or N001_LALA) or antigen binding fragment thereof is administered about every two weeks. In some embodiments, provided herein are methods of treating heart failure (e.g., HFrEF) in a subject in need thereof, the method comprising administering to the subject about 10 mg, about 15 mg, about 20 mg, about 25 mg, about 30 mg, about 35 mg, about 40 mg, about 45 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 350 mg, about 400 mg, about 450 mg, about 500 mg, about 550 mg, about 600 mg, about 650 mg, or about 700 mg of an anti-NPR1 antibody (e.g., N001_DAPA or N001_LALA) or antigen binding fragment thereof, wherein the anti-NPR1 antibody (e.g., N001_DAPA or N001_LALA) or antigen binding fragment thereof is administered about every three weeks. In some embodiments, provided herein are methods of treating heart failure (e.g., HFrEF) in a subject in need thereof, the method comprising administering to the subject about 10 mg, about 15 mg, about 20 mg, about 25 mg, about 30 mg, about 35 mg, about 40 mg, about 45 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 350 mg, about 400 mg, about 450 mg, about 500 mg, about 550 mg, about 600 mg, about 650 mg, or about 700 mg of an anti-NPR1 antibody (e.g., N001_DAPA or N001_LALA) or antigen binding fragment thereof, wherein the anti-NPR1 antibody (e.g., N001_DAPA or N001_LALA) or antigen binding fragment thereof is administered about every four weeks. In some embodiments, provided herein are methods of treating heart failure (e.g., HFrEF) in a subject in need thereof, the method comprising administering to the subject about 10 mg, about 15 mg, about 20 mg, about 25 mg, about 30 mg, about 35 mg, about 40 mg, about 45 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 350 mg, about 400 mg, about 450 mg, about 500 mg, about 550 mg, about 600 mg, about 650 mg, or about 700 mg of an anti-NPR1 antibody (e.g., N001_DAPA or N001_LALA) or antigen binding fragment thereof, wherein the anti-NPR1 antibody (e.g., N001_DAPA or N001_LALA) or antigen binding fragment thereof is administered about every five weeks. In some embodiments, provided herein are methods of treating heart failure (e.g., HFrEF) in a subject in need thereof, the method comprising administering to the subject about 10 mg, about 15 mg, about 20 mg, about 25 mg, about 30 mg, about 35 mg, about 40 mg, about 45 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 350 mg, about 400 mg, about 450 mg, about 500 mg, about 550 mg, about 600 mg, about 650 mg, or about 700 mg of an anti-NPR1 antibody (e.g., N001_DAPA or N001_LALA) or antigen binding fragment thereof, wherein the anti-NPR1 antibody (e.g., N001_DAPA or N001_LALA) or antigen binding fragment thereof is administered about every six weeks. In some embodiments, provided herein are methods of treating heart failure (e.g., HFrEF) in a subject in need thereof, the method comprising administering to the subject about 10 mg, about 15 mg, about 20 mg, about 25 mg, about 30 mg, about 35 mg, about 40 mg, about 45 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 350 mg, about 400 mg, about 450 mg, about 500 mg, about 550 mg, about 600 mg, about 650 mg, or about 700 mg of an anti-NPR1 antibody (e.g., N001_DAPA or N001_LALA) or antigen binding fragment thereof, wherein the anti-NPR1 antibody (e.g., N001_DAPA or N001_LALA) or antigen binding fragment thereof is administered about every seven weeks. In some embodiments, provided herein are methods of treating heart failure (e.g., HFrEF) in a subject in need thereof, the method comprising administering to the subject about 10 mg, about 15 mg, about 20 mg, about 25 mg, about 30 mg, about 35 mg, about 40 mg, about 45 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 350 mg, about 400 mg, about 450 mg, about 500 mg, about 550 mg, about 600 mg, about 650 mg, or about 700 mg of an anti-NPR1 antibody (e.g., N001_DAPA or N001_LALA) or antigen binding fragment thereof, wherein the anti-NPR1 antibody (e.g., N001_DAPA or N001_LALA) or antigen binding fragment thereof is administered about every eight weeks. In some embodiments, provided herein are methods of treating heart failure (e.g., HFrEF) in a subject in need thereof, the method comprising administering to the subject about 10 mg, about 15 mg, about 20 mg, about 25 mg, about 30 mg, about 35 mg, about 40 mg, about 45 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 350 mg, about 400 mg, about 450 mg, about 500 mg, about 550 mg, about 600 mg, about 650 mg, or about 700 mg of an anti-NPR1 antibody (e.g., N001_DAPA or N001_LALA) or antigen binding fragment thereof, wherein the anti-NPR1 antibody (e.g., N001_DAPA or N001_LALA) or antigen binding fragment thereof is administered about every nine weeks. In some embodiments, provided herein are methods of treating heart failure (e.g., HFrEF) in a subject in need thereof, the method comprising administering to the subject about 10 mg, about 15 mg, about 20 mg, about 25 mg, about 30 mg, about 35 mg, about 40 mg, about 45 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 350 mg, about 400 mg, about 450 mg, about 500 mg, about 550 mg, about 600 mg, about 650 mg, or about 700 mg of an anti-NPR1 antibody (e.g., N001_DAPA or N001_LALA) or antigen binding fragment thereof, wherein the anti-NPR1 antibody (e.g., N001_DAPA or N001_LALA) or antigen binding fragment thereof is administered about every ten weeks. In some embodiments, provided herein are methods of treating heart failure (e.g., HFrEF) in a subject in need thereof, the method comprising administering to the subject about 10 mg, about 15 mg, about 20 mg, about 25 mg, about 30 mg, about 35 mg, about 40 mg, about 45 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 350 mg, about 400 mg, about 450 mg, about 500 mg, about 550 mg, about 600 mg, about 650 mg, or about 700 mg of an anti-NPR1 antibody (e.g., N001_DAPA or N001_LALA) or antigen binding fragment thereof, wherein the anti-NPR1 antibody (e.g., N001_DAPA or N001_LALA) or antigen binding fragment thereof is administered about every eleven weeks. In some embodiments, provided herein are methods of treating heart failure (e.g., HFrEF) in a subject in need thereof, the method comprising administering to the subject about 10 mg, about 15 mg, about 20 mg, about 25 mg, about 30 mg, about 35 mg, about 40 mg, about 45 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 350 mg, about 400 mg, about 450 mg, about 500 mg, about 550 mg, about 600 mg, about 650 mg, or about 700 mg of an anti-NPR1 antibody (e.g., N001_DAPA or N001_LALA) or antigen binding fragment thereof, wherein the anti-NPR1 antibody (e.g., N001_DAPA or N001_LALA) or antigen binding fragment thereof is administered about every twelve weeks.

In some embodiments, provided herein are methods of treating heart failure in a subject in need thereof, the method comprising administering to the subject about 60 mg of an anti-NPR1 antibody (e.g., N001_DAPA or N001_LALA) or antigen binding fragment thereof, wherein the anti-NPR1 antibody or antigen binding fragment thereof is administered about once every week, about once every 2 weeks, about once every 3 weeks, about once every 4 weeks, about once every 5 weeks, about once every 6 weeks, about once every 7 weeks, about once every 8 weeks, about once every 9 weeks, about once every 10 weeks, about once every 11 weeks, or about once every 12 weeks. In some embodiments, provided herein are methods of treating heart failure in a subject in need thereof, the method comprising administering to the subject about 120 mg of an anti-NPR1 antibody (e.g., N001_DAPA or N001_LALA) or antigen binding fragment thereof, wherein the anti-NPR1 antibody or antigen binding fragment thereof is administered about once every week, about once every 2 weeks, about once every 3 weeks, about once every 4 weeks, about once every 5 weeks, about once every 6 weeks, about once every 7 weeks, about once every 8 weeks, about once every 9 weeks, about once every 10 weeks, about once every 11 weeks, or about once every 12 weeks. In some embodiments, provided herein are methods of treating heart failure in a subject in need thereof, the method comprising administering to the subject about 240 mg of an anti-NPR1 antibody (e.g., N001_DAPA or N001_LALA) or antigen binding fragment thereof, wherein the anti-NPR1 antibody or antigen binding fragment thereof is administered about once every week, about once every 2 weeks, about once every 3 weeks, about once every 4 weeks, about once every 5 weeks, about once every 6 weeks, about once every 7 weeks, about once every 8 weeks, about once every 9 weeks, about once every 10 weeks, about once every 11 weeks, or about once every 12 weeks.

In some embodiments, provided herein are methods of treating heart failure with reduced ejection fraction (HFrEF) in a subject in need thereof, the method comprising administering to the subject about 60 mg of an anti-NPR1 antibody (e.g., N001_DAPA or N001_LALA) or antigen binding fragment thereof, wherein the anti-NPR1 antibody or antigen binding fragment thereof is administered about once every week, about once every 2 weeks, about once every 3 weeks, about once every 4 weeks, about once every 5 weeks, about once every 6 weeks, about once every 7 weeks, about once every 8 weeks, about once every 9 weeks, about once every 10 weeks, about once every 11 weeks, or about once every 12 weeks. In some embodiments, provided herein are methods of treating heart failure with reduced ejection fraction (HFrEF) in a subject in need thereof, the method comprising administering to the subject about 120 mg of an anti-NPR1 antibody (e.g., N001_DAPA or N001_LALA) or antigen binding fragment thereof, wherein the anti-NPR1 antibody or antigen binding fragment thereof is administered about once every week, about once every 2 weeks, about once every 3 weeks, about once every 4 weeks, about once every 5 weeks, about once every 6 weeks, about once every 7 weeks, about once every 8 weeks, about once every 9 weeks, about once every 10 weeks, about once every 11 weeks, or about once every 12 weeks. In some embodiments, provided herein are methods of treating heart failure with reduced ejection fraction (HFrEF) in a subject in need thereof, the method comprising administering to the subject about 240 mg of an anti-NPR1 antibody (e.g., N001_DAPA or N001_LALA) or antigen binding fragment thereof, wherein the anti-NPR1 antibody or antigen binding fragment thereof is administered about once every week, about once every 2 weeks, about once every 3 weeks, about once every 4 weeks, about once every 5 weeks, about once every 6 weeks, about once every 7 weeks, about once every 8 weeks, about once every 9 weeks, about once every 10 weeks, about once every 11 weeks, or about once every 12 weeks.

In some embodiments, provided herein are methods of treating heart failure with mid-range ejection fraction or heart failure with mildly reduced ejection fraction (HFmrEF) in a subject in need thereof, the method comprising administering to the subject about 60 mg of an anti-NPR1 antibody (e.g., N001_DAPA or N001_LALA) or antigen binding fragment thereof, wherein the anti-NPR1 antibody or antigen binding fragment thereof is administered about once every week, about once every 2 weeks, about once every 3 weeks, about once every 4 weeks, about once every 5 weeks, about once every 6 weeks, about once every 7 weeks, about once every 8 weeks, about once every 9 weeks, about once every 10 weeks, about once every 11 weeks, or about once every 12 weeks. In some embodiments, provided herein are methods of treating heart failure with mid-range ejection fraction or heart failure with mildly reduced ejection fraction (HFmrEF) in a subject in need thereof, the method comprising administering to the subject about 120 mg of an anti-NPR1 antibody (e.g., N001_DAPA or N001_LALA) or antigen binding fragment thereof, wherein the anti-NPR1 antibody or antigen binding fragment thereof is administered about once every week, about once every 2 weeks, about once every 3 weeks, about once every 4 weeks, about once every 5 weeks, about once every 6 weeks, about once every 7 weeks, about once every 8 weeks, about once every 9 weeks, about once every 10 weeks, about once every 11 weeks, or about once every 12 weeks. In some embodiments, provided herein are methods of treating heart failure with mid-range ejection fraction or heart failure with mildly reduced ejection fraction (HFmrEF) in a subject in need thereof, the method comprising administering to the subject about 240 mg of an anti-NPR1 antibody (e.g., N001_DAPA or N001_LALA) or antigen binding fragment thereof, wherein the anti-NPR1 antibody or antigen binding fragment thereof is administered about once every week, about once every 2 weeks, about once every 3 weeks, about once every 4 weeks, about once every 5 weeks, about once every 6 weeks, about once every 7 weeks, about once every 8 weeks, about once every 9 weeks, about once every 10 weeks, about once every 11 weeks, or about once every 12 weeks.

In certain embodiments, provided herein are methods of treating a disorder or a disease associated with natriuretic peptide receptor 1 (NPR1) activity (e.g., heart failure (e.g., heart failure with reduced ejection fraction (HFrEF); or heart failure with mid-range ejection fraction or heart failure with mildly reduced ejection fraction (HFmrEF))) in a subject in need thereof, the method comprising administering to the subject different amounts of an anti-NPR1 antibody (e.g., N001_DAPA or N001_LALA) or antigen binding fragment thereof using an uptitration regimen. In such a regimen, a patient would receive an initial dose that is lower than a secondary or subsequent dose. In certain embodiments, the patient could also receive a tertiary dose that is higher than the secondary dose. In certain embodiments, the patient could also receive a quaternary dose that is higher than the tertiary dose. In certain embodiments, the patient could also receive one or more subsequent dose amounts (e.g., quinary, septenary, octonary, etc.) that are each higher (e.g., in mg) than the preceding dose amount. In certain embodiments, the increases in dose would reach a maximum dose level and would be maintained at that amount.

In certain embodiments, provided herein are methods of treating a disorder or a disease associated with natriuretic peptide receptor 1 (NPR1) activity (e.g., heart failure (e.g., heart failure with reduced ejection fraction (HFrEF); or heart failure with mid-range ejection fraction or heart failure with mildly reduced ejection fraction (HFmrEF))) in a subject in need thereof, the method comprising administering to the subject different amounts of an anti-NPR1 antibody (e.g., N001_DAPA or N001_LALA) or antigen binding fragment thereof. As a non-limiting set of examples, about 10 mg, about 15 mg, about 20 mg, about 25 mg, about 30 mg, about 35 mg, about 40 mg, about 45 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, or about 200 mg would be administered initially, and a secondary or subsequent administration would be at a higher amount, including, e.g., including about 15 mg, about 20 mg, about 25 mg, about 30 mg, about 35 mg, about 40 mg, about 45 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 350 mg, about 400 mg, about 450 mg, about 500 mg, about 550 mg, about 600 mg, about 650 mg, or about 700 mg of the anti-NPR1 antibody or antigen binding fragment thereof; as long as the secondary dose was greater than the initial dose.

In one embodiment, the initial, secondary, tertiary, quaternary, or further doses could be administered, for example, to the patient with heart failure (e.g., heart failure with reduced ejection fraction (HFrEF); or heart failure with mid-range ejection fraction or heart failure with mildly reduced ejection fraction (HFmrEF)) every week for about one week, about two weeks, about three weeks, about four weeks, about five weeks, about six weeks, about seven weeks, or about eight weeks. In one embodiment, the initial, secondary, tertiary, quaternary, or further doses could be administered, for example, to the patient with heart failure (e.g., heart failure with reduced ejection fraction (HFrEF); or heart failure with mid-range ejection fraction or heart failure with mildly reduced ejection fraction (HFmrEF)) every two weeks for about two weeks, about four weeks, about six weeks, about eight weeks, or about ten weeks. In one embodiment, the initial, secondary, tertiary, quaternary, or further doses could be administered, for example, to the patient with heart failure (e.g., heart failure with reduced ejection fraction (HFrEF); or heart failure with mid-range ejection fraction or heart failure with mildly reduced ejection fraction (HFmrEF)) every two weeks for about two weeks, about four weeks, about six weeks, about eight weeks, or about ten weeks. In one embodiment, the initial, secondary, tertiary, quaternary, or further doses could be administered, for example, to the patient with heart failure (e.g., heart failure with reduced ejection fraction (HFrEF); or heart failure with mid-range ejection fraction or heart failure with mildly reduced ejection fraction (HFmrEF)) every three weeks for about three weeks, about six weeks, about nine weeks, about twelve weeks, or about fifteen weeks. In one embodiment, the initial, secondary, tertiary, quaternary, or further doses could be administered, for example, to the patient with heart failure (e.g., heart failure with reduced ejection fraction (HFrEF); or heart failure with mid-range ejection fraction or heart failure with mildly reduced ejection fraction (HFmrEF)) every month for about one month, about two months, about three months, about four months, about five months, about six months, about seven months, or about eight months.

In certain embodiments, the periods for each dosage amount might not be equal. For example, an initial dose (e.g., about 30 mg) could be administered at day 1; a secondary dose (e.g., about 60 mg) could be administered at week 2; and a tertiary dose (e.g., about 120 mg) could be administered once each month after that (i.e., at week 4, week 8, week 12, etc.).

As a non-limiting example, in certain embodiments a patient with heart failure (e.g., heart failure with reduced ejection fraction (HFrEF); or heart failure with mid-range ejection fraction or heart failure with mildly reduced ejection fraction (HFmrEF)) could be administered an initial dose of about 30 mg, and could be administered the initial dose one or more (e.g., two, three, four, or five) times. Such a patient could then be administered a secondary dose of about 60 mg, and could be administered the secondary dose one or more (e.g., two, three, four, or five) times; or could be maintained on this dose for as long as necessary.

As a non-limiting example, in certain embodiments a patient with heart failure (e.g., heart failure with reduced ejection fraction (HFrEF); or heart failure with mid-range ejection fraction or heart failure with mildly reduced ejection fraction (HFmrEF)) could be administered an initial dose of about 30 mg, and could be administered the initial dose one or more (e.g., two, three, four, or five) times. Such a patient could then be administered a secondary dose of about 60 mg, and could be administered the secondary dose one or more (e.g., two, three, four, or five) times. Such a patient could then be administered a tertiary dose of about 120 mg, and could be administered the tertiary dose one or more (e.g., two, three, four, or five) times. Such a patient could then be administered a quaternary dose of about 240 mg, and could be administered the quaternary dose one or more (e.g., two, three, four, or five) times; or could be maintained on this dose for as long as necessary.

As a non-limiting example, in certain embodiments a patient with heart failure (e.g., heart failure with reduced ejection fraction (HFrEF); or heart failure with mid-range ejection fraction or heart failure with mildly reduced ejection fraction (HFmrEF)) could be administered an initial dose of about 30 mg, and could be administered the initial dose one or more (e.g., two, three, four, or five) times. Such a patient could then be administered a secondary dose of about 60 mg, and could be administered the secondary dose one or more (e.g., two, three, four, or five) times. Such a patient could then be administered a tertiary dose of about 120 mg, and could be administered the tertiary dose one or more (e.g., two, three, four, or five) times; or could be maintained on this dose for as long as necessary.

As a non-limiting example, in certain embodiments a patient with heart failure (e.g., heart failure with reduced ejection fraction (HFrEF); or heart failure with mid-range ejection fraction or heart failure with mildly reduced ejection fraction (HFmrEF)) could be administered an initial dose of about 60 mg, and could be administered the initial dose one or more (e.g., two, three, four, or five) times. Such a patient could then be administered a secondary dose of about 120 mg, and could be administered the secondary dose one or more (e.g., two, three, four, or five) times; or could be maintained on this dose for as long as necessary.

As a non-limiting example, in certain embodiments a patient with heart failure (e.g., heart failure with reduced ejection fraction (HFrEF); or heart failure with mid-range ejection fraction or heart failure with mildly reduced ejection fraction (HFmrEF)) could be administered an initial dose of about 60 mg, and could be administered the initial dose one or more (e.g., two, three, four, or five) times. Such a patient could then be administered a secondary dose of about 120 mg, and could be administered the secondary dose one or more (e.g., two, three, four, or five) times. Such a patient could then be administered a tertiary dose of about 240 mg, and could be administered the tertiary dose one or more (e.g., two, three, four, or five) times; or could be maintained on this dose for as long as necessary.

As a non-limiting example, in certain embodiments a patient with heart failure (e.g., heart failure with reduced ejection fraction (HFrEF); or heart failure with mid-range ejection fraction or heart failure with mildly reduced ejection fraction (HFmrEF)) could be administered an initial dose of about 120 mg, and could be administered the initial dose one or more (e.g., two, three, four, or five) times. Such a patient could then be administered a secondary dose of about 240 mg, and could be administered the secondary dose one or more (e.g., two, three, four, or five) times; or could be maintained on this dose for as long as necessary.

As a non-limiting example, in certain embodiments a patient with heart failure (e.g., heart failure with reduced ejection fraction (HFrEF); or heart failure with mid-range ejection fraction or heart failure with mildly reduced ejection fraction (HFmrEF)) could be administered an initial dose of about 60 mg, and could be administered the initial dose one or more (e.g., two, three, four, or five) times. Such a patient could then be administered a secondary dose of about 240 mg, and could be administered the secondary dose one or more (e.g., two, three, four, or five) times; or could be maintained on this dose for as long as necessary.

In certain embodiments the anti-NPR1 antibody could be administered to a patient with heart failure (e.g., heart failure with reduced ejection fraction (HFrEF); or heart failure with mid-range ejection fraction or heart failure with mildly reduced ejection fraction (HFmrEF)) at an initial dose of about 30 mg at day 1 and week 2; and a secondary dose of about 60 mg every four weeks thereafter (i.e., at week 4, week 8, week 12, etc.).

In certain embodiments the anti-NPR1 antibody could be administered to a patient with heart failure (e.g., heart failure with reduced ejection fraction (HFrEF); or heart failure with mid-range ejection fraction or heart failure with mildly reduced ejection fraction (HFmrEF)) at an initial dose of about 60 mg at day 1 and week 2; and a secondary dose of about 120 mg every four weeks thereafter (i.e., at week 4, week 8, week 12, etc.).

In certain embodiments the anti-NPR1 antibody could be administered to a patient with heart failure (e.g., heart failure with reduced ejection fraction (HFrEF); or heart failure with mid-range ejection fraction or heart failure with mildly reduced ejection fraction (HFmrEF)) at an initial dose of about 30 mg at day 1; a secondary dose of about 60 mg at week 2; and a tertiary dose of about 120 mg every four weeks thereafter (i.e., at week 4, week 8, week 12, etc.).

In certain embodiments the anti-NPR1 antibody could be administered to a patient with heart failure (e.g., heart failure with reduced ejection fraction (HFrEF); or heart failure with mid-range ejection fraction or heart failure with mildly reduced ejection fraction (HFmrEF)) at an initial dose of about 60 mg at day 1; and a secondary dose of about 120 mg every four weeks thereafter (i.e., at week 4, week 8, week 12, etc.).

In certain embodiments the anti-NPR1 antibody could be administered to a patient with heart failure (e.g., heart failure with reduced ejection fraction (HFrEF); or heart failure with mid-range ejection fraction or heart failure with mildly reduced ejection fraction (HFmrEF)) at an initial dose of about 120 mg at day 1; and a secondary dose of about 240 mg every four weeks thereafter (i.e., at week 4, week 8, week 12, etc.).

In certain embodiments the anti-NPR1 antibody could be administered to a patient with heart failure (e.g., heart failure with reduced ejection fraction (HFrEF); or heart failure with mid-range ejection fraction or heart failure with mildly reduced ejection fraction (HFmrEF)) at an initial dose of about 30 mg at day 1; a secondary dose of about 60 mg at week 2; a tertiary dose of about 120 mg at week 4; and a quaternary dose of about 240 mg every four weeks thereafter (i.e., at week 8, week 12, etc.).

In certain embodiments, provided herein are methods of treating heart failure (e.g., heart failure with reduced ejection fraction (HFrEF); or heart failure with mid-range ejection fraction or heart failure with mildly reduced ejection fraction (HFmrEF)) in a subject in need thereof, the method comprising administering to the subject different amounts of an anti-NPR1 antibody (e.g., N001_DAPA or N001_LALA) or antigen binding fragment thereof. As a non-limiting set of examples, about 10 mg, about 15 mg, about 20 mg, about 25 mg, about 30 mg, about 35 mg, about 40 mg, about 45 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, or about 200 mg could be administered initially, and a secondary or subsequent administration would be at a higher amount, including, e.g., including about 210 mg, about 220 mg, about 230 mg, about 240 mg, about 250 mg, about 260 mg, about 270 mg, about 280 mg, about 290 mg, about 300 mg, about 350 mg, about 400 mg, about 450 mg, about 500 mg, about 550 mg, about 600 mg, about 650 mg, or about 700 mg of the anti-NPR1 antibody or antigen binding fragment thereof. In one embodiment, about 120 mg of the anti-NPR1 antibody is administered in one or more initial doses (e.g., a first or second dose), and about 240 mg of the anti-NPR 1 antibody or antigen binding fragment thereof is administered in a secondary or subsequent dose (e.g., a second dose, a third dose, or further doses).

In some embodiments, provided herein are methods of treating hypertension (e.g., rHTN) in a subject in need thereof, the method comprising administering to the subject about 10 mg, about 15 mg, about 20 mg, about 25 mg, about 30 mg, about 35 mg, about 40 mg, about 45 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 350 mg, about 400 mg, about 450 mg, about 500 mg, about 550 mg, about 600 mg, about 650 mg, or about 700 mg of an anti-NPR1 antibody (e.g., N001_DAPA or N001_LALA) or antigen binding fragment thereof, wherein the anti-NPR1 antibody (e.g., N001_DAPA or N001_LALA) or antigen binding fragment thereof is administered about every week. In some embodiments, provided herein are methods of treating hypertension (e.g., rHTN) in a subject in need thereof, the method comprising administering to the subject about 10 mg, about 15 mg, about 20 mg, about 25 mg, about 30 mg, about 35 mg, about 40 mg, about 45 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 350 mg, about 400 mg, about 450 mg, about 500 mg, about 550 mg, about 600 mg, about 650 mg, or about 700 mg of an anti-NPR1 antibody (e.g., N001_DAPA or N001_LALA) or antigen binding fragment thereof, wherein the anti-NPR1 antibody (e.g., N001_DAPA or N001_LALA) or antigen binding fragment thereof is administered about every two weeks. In some embodiments, provided herein are methods of treating hypertension (e.g., rHTN) in a subject in need thereof, the method comprising administering to the subject about 10 mg, about 15 mg, about 20 mg, about 25 mg, about 30 mg, about 35 mg, about 40 mg, about 45 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 350 mg, about 400 mg, about 450 mg, about 500 mg, about 550 mg, about 600 mg, about 650 mg, or about 700 mg of an anti-NPR1 antibody (e.g., N001_DAPA or N001_LALA) or antigen binding fragment thereof, wherein the anti-NPR1 antibody (e.g., N001_DAPA or N001_LALA) or antigen binding fragment thereof is administered about every three weeks. In some embodiments, provided herein are methods of treating hypertension (e.g., rHTN) in a subject in need thereof, the method comprising administering to the subject about 10 mg, about 15 mg, about 20 mg, about 25 mg, about 30 mg, about 35 mg, about 40 mg, about 45 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 350 mg, about 400 mg, about 450 mg, about 500 mg, about 550 mg, about 600 mg, about 650 mg, or about 700 mg of an anti-NPR1 antibody (e.g., N001_DAPA or N001_LALA) or antigen binding fragment thereof, wherein the anti-NPR1 antibody (e.g., N001_DAPA or N001_LALA) or antigen binding fragment thereof is administered about every four weeks. In some embodiments, provided herein are methods of treating hypertension (e.g., rHTN) in a subject in need thereof, the method comprising administering to the subject about 10 mg, about 15 mg, about 20 mg, about 25 mg, about 30 mg, about 35 mg, about 40 mg, about 45 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 350 mg, about 400 mg, about 450 mg, about 500 mg, about 550 mg, about 600 mg, about 650 mg, or about 700 mg of an anti-NPR1 antibody (e.g., N001_DAPA or N001_LALA) or antigen binding fragment thereof, wherein the anti-NPR1 antibody (e.g., N001_DAPA or N001_LALA) or antigen binding fragment thereof is administered about every five weeks. In some embodiments, provided herein are methods of treating hypertension (e.g., rHTN) in a subject in need thereof, the method comprising administering to the subject about 10 mg, about 15 mg, about 20 mg, about 25 mg, about 30 mg, about 35 mg, about 40 mg, about 45 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 350 mg, about 400 mg, about 450 mg, about 500 mg, about 550 mg, about 600 mg, about 650 mg, or about 700 mg of an anti-NPR1 antibody (e.g., N001_DAPA or N001_LALA) or antigen binding fragment thereof, wherein the anti-NPR1 antibody (e.g., N001_DAPA or N001_LALA) or antigen binding fragment thereof is administered about every six weeks. In some embodiments, provided herein are methods of treating hypertension (e.g., rHTN) in a subject in need thereof, the method comprising administering to the subject about 10 mg, about 15 mg, about 20 mg, about 25 mg, about 30 mg, about 35 mg, about 40 mg, about 45 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 350 mg, about 400 mg, about 450 mg, about 500 mg, about 550 mg, about 600 mg, about 650 mg, or about 700 mg of an anti-NPR1 antibody (e.g., N001_DAPA or N001_LALA) or antigen binding fragment thereof, wherein the anti-NPR1 antibody (e.g., N001_DAPA or N001_LALA) or antigen binding fragment thereof is administered about every seven weeks. In some embodiments, provided herein are methods of treating hypertension (e.g., rHTN) in a subject in need thereof, the method comprising administering to the subject about 10 mg, about 15 mg, about 20 mg, about 25 mg, about 30 mg, about 35 mg, about 40 mg, about 45 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 350 mg, about 400 mg, about 450 mg, about 500 mg, about 550 mg, about 600 mg, about 650 mg, or about 700 mg of an anti-NPR1 antibody (e.g., N001_DAPA or N001_LALA) or antigen binding fragment thereof, wherein the anti-NPR1 antibody (e.g., N001_DAPA or N001_LALA) or antigen binding fragment thereof is administered about every eight weeks. In some embodiments, provided herein are methods of treating hypertension (e.g., rHTN) in a subject in need thereof, the method comprising administering to the subject about 10 mg, about 15 mg, about 20 mg, about 25 mg, about 30 mg, about 35 mg, about 40 mg, about 45 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 350 mg, about 400 mg, about 450 mg, about 500 mg, about 550 mg, about 600 mg, about 650 mg, or about 700 mg of an anti-NPR1 antibody (e.g., N001_DAPA or N001_LALA) or antigen binding fragment thereof, wherein the anti-NPR1 antibody (e.g., N001_DAPA or N001_LALA) or antigen binding fragment thereof is administered about every nine weeks. In some embodiments, provided herein are methods of treating hypertension (e.g., rHTN) in a subject in need thereof, the method comprising administering to the subject about 10 mg, about 15 mg, about 20 mg, about 25 mg, about 30 mg, about 35 mg, about 40 mg, about 45 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 350 mg, about 400 mg, about 450 mg, about 500 mg, about 550 mg, about 600 mg, about 650 mg, or about 700 mg of an anti-NPR1 antibody (e.g., N001_DAPA or N001_LALA) or antigen binding fragment thereof, wherein the anti-NPR1 antibody (e.g., N001_DAPA or N001_LALA) or antigen binding fragment thereof is administered about every ten weeks. In some embodiments, provided herein are methods of treating hypertension (e.g., rHTN) in a subject in need thereof, the method comprising administering to the subject about 10 mg, about 15 mg, about 20 mg, about 25 mg, about 30 mg, about 35 mg, about 40 mg, about 45 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 350 mg, about 400 mg, about 450 mg, about 500 mg, about 550 mg, about 600 mg, about 650 mg, or about 700 mg of an anti-NPR1 antibody (e.g., N001_DAPA or N001_LALA) or antigen binding fragment thereof, wherein the anti-NPR1 antibody (e.g., N001_DAPA or N001_LALA) or antigen binding fragment thereof is administered about every eleven weeks. In some embodiments, provided herein are methods of treating hypertension (e.g., rHTN) in a subject in need thereof, the method comprising administering to the subject about 10 mg, about 15 mg, about 20 mg, about 25 mg, about 30 mg, about 35 mg, about 40 mg, about 45 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 350 mg, about 400 mg, about 450 mg, about 500 mg, about 550 mg, about 600 mg, about 650 mg, or about 700 mg of an anti-NPR1 antibody (e.g., N001_DAPA or N001_LALA) or antigen binding fragment thereof, wherein the anti-NPR1 antibody (e.g., N001_DAPA or N001_LALA) or antigen binding fragment thereof is administered about every twelve weeks.

In some embodiments, provided herein are methods of treating hypertension in a subject in need thereof, the method comprising administering to the subject about 60 mg of an anti-NPR1 antibody (e.g., N001_DAPA or N001_LALA) or antigen binding fragment thereof, wherein the anti-NPR1 antibody or antigen binding fragment thereof is administered about once every week, about once every 2 weeks, about once every 3 weeks, about once every 4 weeks, about once every 5 weeks, about once every 6 weeks, about once every 7 weeks, about once every 8 weeks, about once every 9 weeks, about once every 10 weeks, about once every 11 weeks, or about once every 12 weeks. In some embodiments, provided herein are methods of treating hypertension in a subject in need thereof, the method comprising administering to the subject about 120 mg of an anti-NPR1 antibody (e.g., N001_DAPA or N001_LALA) or antigen binding fragment thereof, wherein the anti-NPR1 antibody or antigen binding fragment thereof is administered about once every week, about once every 2 weeks, about once every 3 weeks, about once every 4 weeks, about once every 5 weeks, about once every 6 weeks, about once every 7 weeks, about once every 8 weeks, about once every 9 weeks, about once every 10 weeks, about once every 11 weeks, or about once every 12 weeks. In some embodiments, provided herein are methods of treating hypertension in a subject in need thereof, the method comprising administering to the subject about 240 mg of an anti-NPR1 antibody (e.g., N001_DAPA or N001_LALA) or antigen binding fragment thereof, wherein the anti-NPR1 antibody or antigen binding fragment thereof is administered about once every week, about once every 2 weeks, about once every 3 weeks, about once every 4 weeks, about once every 5 weeks, about once every 6 weeks, about once every 7 weeks, about once every 8 weeks, about once every 9 weeks, about once every 10 weeks, about once every 11 weeks, or about once every 12 weeks.

In some embodiments, provided herein are methods of treating resistant hypertension in a subject in need thereof, the method comprising administering to the subject about 60 mg of an anti-NPR1 antibody (e.g., N001_DAPA or N001_LALA) or antigen binding fragment thereof, wherein the anti-NPR1 antibody or antigen binding fragment thereof is administered about once every week, about once every 2 weeks, about once every 3 weeks, about once every 4 weeks, about once every 5 weeks, about once every 6 weeks, about once every 7 weeks, about once every 8 weeks, about once every 9 weeks, about once every 10 weeks, about once every 11 weeks, or about once every 12 weeks. In some embodiments, provided herein are methods of treating resistant hypertension in a subject in need thereof, the method comprising administering to the subject about 120 mg of an anti-NPR1 antibody (e.g., N001_DAPA or N001_LALA) or antigen binding fragment thereof, wherein the anti-NPR1 antibody or antigen binding fragment thereof is administered about once every week, about once every 2 weeks, about once every 3 weeks, about once every 4 weeks, about once every 5 weeks, about once every 6 weeks, about once every 7 weeks, about once every 8 weeks, about once every 9 weeks, about once every 10 weeks, about once every 11 weeks, or about once every 12 weeks. In some embodiments, provided herein are methods of treating resistant hypertension in a subject in need thereof, the method comprising administering to the subject about 240 mg of an anti-NPR1 antibody (e.g., N001_DAPA or N001_LALA) or antigen binding fragment thereof, wherein the anti-NPR1 antibody or antigen binding fragment thereof is administered about once every week, about once every 2 weeks, about once every 3 weeks, about once every 4 weeks, about once every 5 weeks, about once every 6 weeks, about once every 7 weeks, about once every 8 weeks, about once every 9 weeks, about once every 10 weeks, about once every 11 weeks, or about once every 12 weeks.

In certain embodiments, provided herein are methods of treating a disorder or a disease associated with natriuretic peptide receptor 1 (NPR1) activity (e.g., hypertension (e.g., resistant hypertension (rHTN))) in a subject in need thereof, the method comprising administering to the subject different amounts of an anti-NPR1 antibody (e.g., N001_DAPA or N001_LALA) or antigen binding fragment thereof using an uptitration regimen. In such a regimen, a patient would receive an initial dose that is lower than a secondary or subsequent dose. In certain embodiments, the patient could also receive a tertiary dose that is higher than the secondary dose. In certain embodiments, the patient could also receive a quaternary dose that is higher than the tertiary dose. In certain embodiments, the patient could also receive one or more subsequent dose amounts (e.g., quinary, septenary, octonary, etc.) that are each higher (e.g., in mg) than the preceding dose amount. In certain embodiments, the increases in dose would reach a maximum dose level and would be maintained at that amount.

In certain embodiments, provided herein are methods of treating a disorder or a disease associated with natriuretic peptide receptor 1 (NPR1) activity (e.g., hypertension (e.g., resistant hypertension (rHTN))) in a subject in need thereof, the method comprising administering to the subject different amounts of an anti-NPR1 antibody (e.g., N001_DAPA or N001_LALA) or antigen binding fragment thereof. As a non-limiting set of examples, about 10 mg, about 15 mg, about 20 mg, about 25 mg, about 30 mg, about 35 mg, about 40 mg, about 45 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, or about 200 mg would be administered initially, and a secondary or subsequent administration would be at a higher amount, including, e.g., including about 15 mg, about 20 mg, about 25 mg, about 30 mg, about 35 mg, about 40 mg, about 45 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 350 mg, about 400 mg, about 450 mg, about 500 mg, about 550 mg, about 600 mg, about 650 mg, or about 700 mg of the anti-NPR1 antibody or antigen binding fragment thereof; as long as the secondary dose was greater than the initial dose.

In one embodiment, the initial, secondary, tertiary, quaternary, or further doses could be administered, for example, to the patient with hypertension (e.g., resistant hypertension (rHTN)) every week for about one week, about two weeks, about three weeks, about four weeks, about five weeks, about six weeks, about seven weeks, or about eight weeks. In one embodiment, the initial, secondary, tertiary, quaternary, or further doses could be administered, for example, to the patient with hypertension (e.g., resistant hypertension (rHTN)) every two weeks for about two weeks, about four weeks, about six weeks, about eight weeks, or about ten weeks. In one embodiment, the initial, secondary, tertiary, quaternary, or further doses could be administered, for example, to the patient with hypertension (e.g., resistant hypertension (rHTN)) every two weeks for about two weeks, about four weeks, about six weeks, about eight weeks, or about ten weeks. In one embodiment, the initial, secondary, tertiary, quaternary, or further doses could be administered, for example, to the patient with hypertension (e.g., resistant hypertension (rHTN)) every three weeks for about three weeks, about six weeks, about nine weeks, about twelve weeks, or about fifteen weeks. In one embodiment, the initial, secondary, tertiary, quaternary, or further doses could be administered, for example, to the patient with hypertension (e.g., resistant hypertension (rHTN)) every month for about one month, about two months, about three months, about four months, about five months, about six months, about seven months, or about eight months.

In certain embodiments, the periods for each dosage amount might not be equal. For example, an initial dose (e.g., about 30 mg) could be administered at day 1; a secondary dose (e.g., about 60 mg) could be administered at week 2; and a tertiary dose (e.g., about 120 mg) could be administered once each month after that (i.e., at week 4, week 8, week 12, etc.).

As a non-limiting example, in certain embodiments a patient with hypertension (e.g., resistant hypertension (rHTN)) could be administered an initial dose of about 30 mg, and could be administered the initial dose one or more (e.g., two, three, four, or five) times. Such a patient could then be administered a secondary dose of about 60 mg, and could be administered the secondary dose one or more (e.g., two, three, four, or five) times; or could be maintained on this dose for as long as necessary.

As a non-limiting example, in certain embodiments a patient with hypertension (e.g., resistant hypertension (rHTN)) could be administered an initial dose of about 30 mg, and could be administered the initial dose one or more (e.g., two, three, four, or five) times. Such a patient could then be administered a secondary dose of about 60 mg, and could be administered the secondary dose one or more (e.g., two, three, four, or five) times. Such a patient could then be administered a tertiary dose of about 120 mg, and could be administered the tertiary dose one or more (e.g., two, three, four, or five) times. Such a patient could then be administered a quaternary dose of about 240 mg, and could be administered the quaternary dose one or more (e.g., two, three, four, or five) times; or could be maintained on this dose for as long as necessary.

As a non-limiting example, in certain embodiments a patient with hypertension (e.g., resistant hypertension (rHTN)) could be administered an initial dose of about 30 mg, and could be administered the initial dose one or more (e.g., two, three, four, or five) times. Such a patient could then be administered a secondary dose of about 60 mg, and could be administered the secondary dose one or more (e.g., two, three, four, or five) times. Such a patient could then be administered a tertiary dose of about 120 mg, and could be administered the tertiary dose one or more (e.g., two, three, four, or five) times; or could be maintained on this dose for as long as necessary.

As a non-limiting example, in certain embodiments a patient with hypertension (e.g., resistant hypertension (rHTN)) could be administered an initial dose of about 60 mg, and could be administered the initial dose one or more (e.g., two, three, four, or five) times. Such a patient could then be administered a secondary dose of about 120 mg, and could be administered the secondary dose one or more (e.g., two, three, four, or five) times; or could be maintained on this dose for as long as necessary.

As a non-limiting example, in certain embodiments a patient with hypertension (e.g., resistant hypertension (rHTN)) could be administered an initial dose of about 60 mg, and could be administered the initial dose one or more (e.g., two, three, four, or five) times. Such a patient could then be administered a secondary dose of about 120 mg, and could be administered the secondary dose one or more (e.g., two, three, four, or five) times. Such a patient could then be administered a tertiary dose of about 240 mg, and could be administered the tertiary dose one or more (e.g., two, three, four, or five) times; or could be maintained on this dose for as long as necessary.

As a non-limiting example, in certain embodiments a patient with hypertension (e.g., resistant hypertension (rHTN)) could be administered an initial dose of about 120 mg, and could be administered the initial dose one or more (e.g., two, three, four, or five) times. Such a patient could then be administered a secondary dose of about 240 mg, and could be administered the secondary dose one or more (e.g., two, three, four, or five) times; or could be maintained on this dose for as long as necessary.

As a non-limiting example, in certain embodiments a patient with hypertension (e.g., resistant hypertension (rHTN)) could be administered an initial dose of about 60 mg, and could be administered the initial dose one or more (e.g., two, three, four, or five) times. Such a patient could then be administered a secondary dose of about 240 mg, and could be administered the secondary dose one or more (e.g., two, three, four, or five) times; or could be maintained on this dose for as long as necessary.

In certain embodiments the anti-NPR1 antibody could be administered to a patient with hypertension (e.g., resistant hypertension (rHTN)) at an initial dose of about 30 mg at day 1 and week 2; and a secondary dose of about 60 mg every four weeks thereafter (i.e., at week 4, week 8, week 12, etc.).

In certain embodiments the anti-NPR1 antibody could be administered to a patient with hypertension (e.g., resistant hypertension (rHTN)) at an initial dose of about 60 mg at day 1 and week 2; and a secondary dose of about 120 mg every four weeks thereafter (i.e., at week 4, week 8, week 12, etc.).

In certain embodiments the anti-NPR1 antibody could be administered to a patient with hypertension (e.g., resistant hypertension (rHTN)) at an initial dose of about 30 mg at day 1; a secondary dose of about 60 mg at week 2; and a tertiary dose of about 120 mg every four weeks thereafter (i.e., at week 4, week 8, week 12, etc.).

In certain embodiments the anti-NPR1 antibody could be administered to a patient with hypertension (e.g., resistant hypertension (rHTN)) at an initial dose of about 60 mg at day 1; and a secondary dose of about 120 mg every four weeks thereafter (i.e., at week 4, week 8, week 12, etc.).

In certain embodiments the anti-NPR1 antibody could be administered to a patient with hypertension (e.g., resistant hypertension (rHTN)) at an initial dose of about 120 mg at day 1; and a secondary dose of about 240 mg every four weeks thereafter (i.e., at week 4, week 8, week 12, etc.).

In certain embodiments the anti-NPR1 antibody could be administered to a patient with hypertension (e.g., resistant hypertension (rHTN)) at an initial dose of about 30 mg at day 1; a secondary dose of about 60 mg at week 2; a tertiary dose of about 120 mg at week 4; and a quaternary dose of about 240 mg every four weeks thereafter (i.e., at week 8, week 12, etc.).

In certain embodiments, provided herein are methods of treating hypertension (e.g., resistant hypertension (rHTN)) in a subject in need thereof, the method comprising administering to the subject different amounts of an anti-NPR1 antibody (e.g., N001_DAPA or N001_LALA) or antigen binding fragment thereof. As a non-limiting set of examples, about 10 mg, about 15 mg, about 20 mg, about 25 mg, about 30 mg, about 35 mg, about 40 mg, about 45 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, or about 200 mg could be administered initially, and a secondary or subsequent administration would be at a higher amount, including, e.g., including about 210 mg, about 220 mg, about 230 mg, about 240 mg, about 250 mg, about 260 mg, about 270 mg, about 280 mg, about 290 mg, about 300 mg, about 350 mg, about 400 mg, about 450 mg, about 500 mg, about 550 mg, about 600 mg, about 650 mg, or about 700 mg of the anti-NPR1 antibody or antigen binding fragment thereof. In one embodiment, about 120 mg of the anti-NPR1 antibody is administered in one or more initial doses (e.g., a first or second dose), and about 240 mg of the anti-NPR 1 antibody or antigen binding fragment thereof is administered in a secondary or subsequent dose (e.g., a second dose, a third dose, or further doses).

Any of the doses, methods, or uses described herein may be administered to a patient receiving one or more additional therapies including, but not limited to, those described herein. As a nonlimiting example, provided herein are methods of treating a cardiovascular disorder (e.g., heart failure such as HFrEF, or hypertension such as resistant hypertension) in a subject in need thereof, the method comprising administering to the subject about 10 mg, about 15 mg, about 20 mg, about 25 mg, about 30 mg, about 35 mg, about 40 mg, about 45 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 350 mg, about 400 mg, about 450 mg, about 500 mg, about 550 mg, about 600 mg, about 650 mg, or about 700 mg of an anti-NPR1 antibody (e.g., N001_DAPA or N001_LALA) or antigen binding fragment thereof, wherein the subject is receiving or being administered one or more additional therapeutically active agents (e.g., an ACE (angiotensin-converting-enzyme) inhibitor, an angiotensin receptor blocker (ARB), a beta blocker, a diuretic, or combinations thereof). In some embodiments, the methods of treating a cardiovascular disorder in a subject in need thereof comprise administering to the subject about comprises administering about 60 mg of an anti-NPR1 antibody (e.g., N001_DAPA or N001_LALA) or antigen binding fragment thereof, wherein the subject is receiving or being administered one or more additional therapeutically active agents (e.g., an ACE (angiotensin-converting-enzyme) inhibitor, an angiotensin receptor blocker (ARB), a beta blocker, a diuretic, or combinations thereof). In some embodiments, the methods of treating a cardiovascular disorder in a subject in need thereof comprise administering to the subject about comprises administering about 120 mg of an anti-NPR1 antibody (e.g., N001_DAPA or N001_LALA) or antigen binding fragment thereof, wherein the subject is receiving or being administered one or more additional therapeutically active agents (e.g., an ACE (angiotensin-converting-enzyme) inhibitor, an angiotensin receptor blocker (ARB), a beta blocker, a diuretic, or combinations thereof). In some embodiments, the methods of treating a cardiovascular disorder in a subject in need thereof comprise administering about 240 mg of an anti-NPR1 antibody (e.g., N001_DAPA or N001_LALA) or antigen binding fragment thereof, wherein the subject is receiving or being administered one or more additional therapeutically active agents (e.g., an ACE (angiotensin-converting-enzyme) inhibitor, an angiotensin receptor blocker (ARB), a beta blocker, a diuretic, or combinations thereof).

The methods and uses described herein in which an anti-NPR1 antibody or antigen binding fragment thereof may result in increasing plasma and urine levels of cGMP and/or blood pressure lowering. These changes can have an effect on plasma biomarkers of heart failure (NT-proBP, ANP, BNP), and urine sodium excretion. These effects may have an impact on clinical symptoms of cardiovascular disorders (e.g., heart failure such as HFrEF) which can be measured, for example, through an assessment of clinical congestion score and/or NYHA class. NPR1 stimulation may also improve arterial stiffness, which can be measured, for example, through pulse pressure.

Also provided herein are compositions, e.g., pharmaceutical compositions, for use in treatment of an NPR1-associated disease. Such compositions include one or more anti-NPR1 antibodies or an antigen-binding fragment thereof as described herein and may include a pharmaceutically acceptable carrier. Such compositions can further include another agent, e.g., a current standard of care for the disease to be treated.

Pharmaceutical compositions typically include a pharmaceutically acceptable carrier. As used herein the term “pharmaceutically acceptable carrier” refers to a carrier or a diluent that does not cause significant irritation to a subject and does not abrogate the biological activity and properties of the administered anti-NPR1 antibody or antigen binding fragment and/or any additional therapeutic agent in the composition. Pharmaceutically acceptable carriers may enhance or stabilize the composition or can be used to facilitate preparation of the composition. Pharmaceutically acceptable carriers may include saline, solvents, dispersion media, coatings, antibacterial and antifungal agents, isotonic and absorption delaying agents, and the like, compatible with pharmaceutical administration. An adjuvant may also be included in any of these formulations. Pharmaceutical compositions are typically formulated to be compatible with its intended route of administration. Examples of routes of administration include parenteral (e.g., intravenous, intraarterial, intraperitoneal), oral, intracranial, intrathecal, or intranasal (e.g., inhalation), intradermal, subcutaneous, or transmucosal administration. In a specific embodiment, any of the anti-NPR1 antibodies or antigen binding fragments thereof may be administered subcutaneously. In a specific embodiment, any of the anti-NPR1 antibodies or antigen binding fragments thereof may be prepared for subcutaneous administration. In some embodiments, the pharmaceutical compositions are formulated to deliver anti-NPR1 antibodies or antigen-binding fragments thereof to cross the blood-brain barrier. The phrases “physiologically acceptable carrier” and “pharmaceutically acceptable carrier” may be used interchangeably.

As used herein, the term “excipient” refers to an inert substance added to a pharmaceutical composition to further facilitate administration of an active ingredient. Formulations for parenteral administration can, for example, contain excipients such as sterile water or saline, polyalkylene glycols such as polyethylene glycol, vegetable oils, or hydrogenated napthalenes. Other exemplary excipients include, but are not limited to, calcium bicarbonate, calcium phosphate, various sugars and types of starch, cellulose derivatives, gelatin, ethylene-vinyl acetate co-polymer particles, and surfactants, including, for example, polysorbate 20.

A pharmaceutical composition as described herein can be administered by a variety of methods known in the art. The route and/or mode of administration may vary depending upon the desired results. In some embodiments, the administration is intravitreal, intravenous, intramuscular, intraperitoneal, or subcutaneous. The pharmaceutically acceptable carrier should be suitable for intravitreal, intravenous, intramuscular, subcutaneous, parenteral, spinal, or epidermal administration (e.g., by injection or infusion). Depending on the route of administration, the active compound(s), i.e., the anti-NPR1 antibody or antigen binding fragment and optionally the additional therapeutic agent, may be coated in a material to protect the compound(s) from the action of acids and other natural conditions that may inactivate the compound(s).

Typically, a therapeutically effective dose or efficacious dose of the anti-NPR1 antibodies or antigen binding fragments is employed in the pharmaceutical compositions described herein. The anti-NPR1 antibodies or antigen binding fragments may be formulated into pharmaceutically acceptable dosage forms by conventional methods known to those of skill in the art.

Methods of formulating suitable pharmaceutical compositions can be any know or used, see, e.g., Remington: The Science and Practice of Pharmacy. 21^{st} ed., 2005; and the books in the series Drugs and the Pharmaceutical Sciences: a Series of Textbooks and Monographs (Dekker, NY), the contents of each of which are incorporated by reference herein for this purpose. For example, solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Dosage regimens for anti-NPR1 antibodies and antigen binding fragments with or without an additional therapeutic agent may be adjusted to provide the optimum desired response (e.g., a therapeutic response). For example, a single bolus of one or both agents may be administered at one time, several divided doses may be administered over a predetermined period of time, or the dose of one or both agents may be proportionally reduced or increased as indicated by the exigencies of the therapeutic situation. For any particular subject, specific dosage regimens may be adjusted over time according to the individual's need, and the professional judgment of the treating clinician. Parenteral compositions may be formulated in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a therapeutic compound (i.e., an effective dosage) depends on the therapeutic compounds selected. The skilled artisan (such as a medical doctor) will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

Dosage, toxicity and therapeutic efficacy of the therapeutic compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

Dosage regimens for anti-NPR1 antibodies and antigen binding fragments alone or in combination with an additional therapeutic agent may be adjusted to provide the optimum desired response (e.g., a therapeutic response). For example, a single bolus of one or both agents may be administered at one time, several divided doses may be administered over a predetermined period of time, or the dose of one or both agents may be proportionally reduced or increased as indicated by the exigencies of the therapeutic situation. For any particular subject, specific dosage regimens may be adjusted over time according to the individual's need, and the professional judgment of the treating clinician. Parenteral compositions may be formulated in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

All the embodiments described herein for uses and methods of treatment according to the present invention are equally applicable to:
the use of any one of the antibodies or antigen binding fragments as described herein for the manufacture of a medicament for use according to the present invention,
the use of any one of the antibodies or antigen binding fragments described herein according to the present invention,
any one of the antibodies or antigen binding fragments described herein for use according to the present invention,
the pharmaceutical compositions comprising any one of the antibodies or antigen binding fragments described herein for the use according to the present invention,
the use of the pharmaceutical compositions comprising any one of the antibodies or antigen binding fragments described herein according to the present invention, and
the use of the pharmaceutical compositions comprising any one of the antibodies or antigen binding fragments described herein for the manufacture of a medicament for use according to the present invention.

### Combination Therapies

The various treatments described herein can be combined with other treatment partners or therapeutic agents such as the current standard of care for a disease associated with NPR1 loss of function, e.g., the current standard of care for one or more of the diseases or disorders discussed herein. For example, the NPR1 antibodies or an antigen-binding fragment thereof described herein can be combined with one or more of an ACE (angiotensin-converting-enzyme) inhibitor, an angiotensin receptor blocker (ARB), a neprilysin inhibitor, a beta blocker, a diuretic, a calcium channel blocker, a cardiac glycoside, a sodium-glucose co-transporter 2 inhibitor (SGLT2i), or combinations thereof. As a non-limiting set of examples, the NPR1 antibody or antigen binding from may be combined with an additional therapeutic agent selected from enalapril, benazepril, captopril, fosinopril, lisinopril, moexipril, perindopril, quinapril, ramipril, trandolapril, valsartan, azilsartan, candesartan, eprosartan, irbesartan, losartan, olmesartan, telmisartan, sacubitril, bisoprolol, carvedilol, propanolol, metoprolol, metoprolol tartrate, metoprolol succinate, thiazide diuretics, loop diuretics, potassium-sparing diuretics, amlodipine, clevidipine, diltiazem, felodipine, isradipine, nicardipine, nifedipine, nisoldipine, verapamil, a digitalis glycoside, canagliflozin, dapagliflozin, empagliflozin, sotagliflozin, ertugliflozin, and combinations thereof. Exemplary diuretics and digitalis glycosides include, but are not limited to, chlorothiazide, chlorthalidone, hydrochlorothiazide, indapamide, metolazone, bumetanide, ethacrynic acid, furosemide, torsemide, amiloride, eplerenone, finerone, spironolactone, triamterene, digoxin, and combinations thereof. In some embodiments, the NPR1 antibodies or an antigen-binding fragment thereof described herein may be combined with an angiotensin receptor neprilysin inhibitor (ARNI) such as a combination of sacubitril and valsartan (e.g., Entresto®). In some embodiments, the NPR1 antibodies or an antigen-binding fragment thereof described herein can be combined with one or more of a corticosteroid (e.g., an inhaled corticosteroid such as fluticasone, budesonide, mometasone, beclomethasone, ciclesonide, or fluticasone furoate; or an oral or intravenous corticosteroid such as prednisone or methylprednisolone), a leukotriene modifier (e.g., montelukast, zafirlukast, or zileuton), a bronchodilator (e.g., a long-acting beta agonist (e.g., salmeterol or formoterol), a short-acting beta agonist (e.g., albuterol or levalbuterol), theophylline or ipratropium), or combinations thereof (e.g., a combination of fluticasone and salmeterol, a combination of budesonide and formoterol, or a combination of formoterol and mometasone). In some embodiments, the NPR1 antibodies or an antigen-binding fragment thereof described herein can be combined with one or more of a beta-adrenoceptor antagonist (e.g., timolol, levobunolol, metipranolol, carteolol, or betaxolol), a carbonic anhydrase inhibitor (e.g., acetazolamide, dorzolamide, brinzolamide, or methazolamide), an alpha 2-adrenoceptor agonist (e.g., brimonidine or apraclonidine), a parasympathomimetic (e.g., cholinomimetics like pilocarpine), a prostaglandin analog (e.g., latanoprost, latanoprostene bunod, travoprost, bimatoprost, or tafluprost), a cardiac-specific myosin activator (e.g., omecamtiv mecarbil (also referred to as CK-1827452)), a cell therapy (e.g., allogeneic cell therapy rexlemestrocel-L (REVASCOR®), CardiAMP™ Cell Therapy), a rho kinase inhibitor (e.g., netarsudil or ripasudil), or combinations thereof (e.g., a combination of rho kinase inhibitor and latanoprost).

Accordingly, the methods of treating a disease associated with NPR1 loss of function described herein can further include administering a second agent to the subject in need of treatment. In some embodiments, the patient or subject may have already been administered one or more additional therapeutic agents one or more times. In certain embodiments, the subject may already be receiving an ACE (angiotensin-converting-enzyme) inhibitor, an angiotensin receptor blocker (ARB), a beta blocker, a diuretic, or combinations thereof. In some embodiments, the subject may have been administered an ACE (angiotensin-converting-enzyme) inhibitor, an angiotensin receptor blocker (ARB), a beta blocker, a diuretic, or combinations thereof for a period of time (e.g., weeks, months, or years) before administration of an anti-NPR1 antibody or antigen binding fragment thereof as described herein. In some embodiments, the subject may be concurrently administered a combination of sacubitril and valsartan (e.g., Entresto®). In some embodiments, the subject may have been administered a combination of sacubitril and valsartan (e.g., Entresto®) for a period of time (e.g., weeks, months, or years) before administration of an anti-NPR1 antibody or antigen binding fragment thereof as described herein. In some embodiments, the subject may be concurrently administered an ACE inhibitor. In some embodiments, the subject may have been administered an ACE inhibitor for a period of time (e.g., weeks, months, or years) before administration of an anti-NPR1 antibody or antigen binding fragment thereof as described herein. In some embodiments, the subject may be concurrently administered an ARB. In some embodiments, the subject may have been administered an ARB for a period of time (e.g., weeks, months, or years) before administration of an anti-NPR1 antibody or antigen binding fragment thereof as described herein. In some embodiments, the subject may be concurrently administered a beta blocker. In some embodiments, the subject may have been administered a beta blocker for a period of time (e.g., weeks, months, or years) before administration of an anti-NPR1 antibody or antigen binding fragment thereof as described herein. In some embodiments, the subject may be concurrently administered a diuretic. In some embodiments, the subject may have been administered a diuretic for a period of time (e.g., weeks, months, or years) before administration of an anti-NPR1 antibody or antigen binding fragment thereof as described herein. In some embodiments, the subject may be concurrently administered an ACE inhibitor, an ARB, a beta blocker, a diuretic, or a combination thereof. In some embodiments, the subject may have been administered an ACE inhibitor, an ARB, a beta blocker, a diuretic, or a combination thereof for a period of time (e.g., weeks, months, or years) before administration of an anti-NPR1 antibody or antigen binding fragment thereof as described herein.

The term “combination” refers to either a fixed combination in one dosage unit form, or a combined administration where an anti-NPR1 antibody or antigen-binding fragment thereof described herein and a combination partner (e.g., another drug as explained below, also referred to as “therapeutic agent” or “co-agent”) may be administered independently at the same time or separately within time intervals, especially where these time intervals allow that the combination partners show a cooperative, e.g., synergistic effect. The single components may be packaged in a kit or separately. One or both of the components (e.g., powders or liquids) may be reconstituted or diluted to a desired dose prior to administration. The terms “co-administration” or “combined administration” or the like as utilized herein are meant to encompass administration of the selected combination partner to a single subject in need thereof (e.g., a patient), and are intended to include treatment regimens in which the agents are not necessarily administered by the same route of administration and/or at the same time. The term “pharmaceutical combination” as used herein means a product that results from the mixing or combining of more than one therapeutic agent and includes both fixed and non-fixed combinations of the therapeutic agents. The term “fixed combination” means that the therapeutic agents, e.g., a compound described herein and a combination partner, are both administered to a patient simultaneously in the form of a single entity or dosage. The term “non-fixed combination” means that the therapeutic agents, e.g., a compound described herein and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g., the administration of three or more therapeutic agent.

The term “pharmaceutical combination” as used herein refers to either a fixed combination in one dosage unit form, or non-fixed combination or a kit of parts for the combined administration where two or more therapeutic agents may be administered independently at the same time or separately within time intervals, especially where these time intervals allow that the combination partners show a cooperative, e.g., synergistic effect.

The term “combination therapy” refers to the administration of two or more therapeutic agents to treat a therapeutic condition or disorder described herein. Such administration encompasses co-administration of these therapeutic agents in a substantially simultaneous manner, such as in a single capsule having a fixed ratio of active ingredients. Alternatively, such administration encompasses co-administration in multiple, or in separate containers (e.g., tablets, capsules, powders, and liquids) for each active ingredient. Powders and/or liquids may be reconstituted or diluted to a desired dose prior to administration. In addition, such administration also encompasses use of each type of therapeutic agent in a sequential manner, either at approximately the same time or at different times. As a non-limiting example, in some embodiments, the anti-NPR1 antibody or antigen binding fragment thereof may be administered on the same day as an ACE inhibitor, an ARB, a beta blocker, a diuretic, or a combination thereof. As a non-limiting example, in some embodiments, the anti-NPR1 antibody or antigen binding fragment thereof may be administered on different days as an ACE inhibitor, an ARB, a beta blocker, a diuretic, or a combination thereof. In either case, the treatment regimen will provide beneficial effects of the drug combination in treating the conditions or disorders described herein.

### Kits

Also provided herein are kits including one or more of the compositions provided herein (e.g., an antibody or antigen binding fragment thereof described in Table 2) and instructions for use in the described methods and uses herein. Instructions for use can include instructions for diagnosis or treatment of an NPR1-associated disease. Kits as provided herein may be used in accordance with any of the methods described herein. Those skilled in the art will be aware of other suitable uses for kits provided herein, and will be able to employ the kits for such uses. Kits as provided herein can also include a mailer (e.g., a postage paid envelope or mailing pack) that can be used to return the sample for analysis, e.g., to a laboratory. The kit can include one or more containers for the sample, or the sample can be in a standard blood collection vial. The kit can also include one or more of an informed consent form, a test requisition form, and instructions on how to use the kit in a method described herein. Methods for using such kits are also included herein. One or more of the forms (e.g., the test requisition form) and the container holding the sample can be coded, for example, with a bar code for identifying the subject who provided the sample.

The described methods and uses may be further illustrated by the following examples and claims, which are illustrative and are not meant to be further limiting. One skilled in the art will recognize many methods and materials similar or equivalent to those described herein, which could be used in the practice of the described compositions and methods. Such equivalents are within the scope of the present description and claims. The contents of all references, including issued patents and published patent applications, cited throughout this application are hereby incorporated by reference.

### EXAMPLES

The following examples provide illustrative embodiments. One of ordinary skill in the art will recognize the numerous modifications and variations that may be performed without altering the spirit or scope of the described methods and uses. Such modifications and variations are encompassed within the scope of the description. The examples provided do not in any way limit the methods and uses described here.

### Example 1: A First in Human (FIH) Single Ascending Dose (SAD) Study in Healthy Participants

Below is described a first-in-human, randomized, sponsor open-label, participant and investigator blinded, placebo-controlled, single ascending dose study to explore safety, tolerability, and pharmacokinetics in healthy participants.

The participants are enrolled in the SAD study and administered an anti-NPR1 antibody as described herein or placebo. In this FIH study, the planned single subcutaneous doses are 1, 3, 10, 30, 60, 120, 240, 450, and 600 mg. Depending on the dosage, the participant may receive between one and four injections of up to 1 ml each (i.e., may receive one injection for the lowest dosages, and up to four injections for the higest dosages). Doses may be modified based on emerging data related to safety, tolerability, PK and PD from preceding doses. Safety, pharmacokinetics, and pharmacodynamics will be assessed over 91 days. The total study duration is 151 days (including the full screening period up to 28 days and 30-day safety follow up call after the end of study visit).

### Key Inclusion Criteria:

Healthy male and female participants age 18 to 50 years of age included, and in good health as determined by past medical history, physical examination, vital signs, electrocardiogram, and laboratory tests at screening.
Participants must weigh at least 50 kg to participate in the study, and must have a body mass index (BMI) within the range of 18-32 kg/m². BMI=Body weight (kg)/[Height (m)]².
For Japanese descent cohort: participants must be defined as being of first, second or third generation Japanese descent origin, with each set of parents qualifying as Japanese under the prior generation. Generations will be defined as follows: ‘First generation’ Japanese are participants who were born in Japan to parents of Japanese ethnicity. ‘Second generation’ Japanese are participants who are born outside of Japan to 1st generation Japanese parents. ‘Third generation’ Japanese are participants who are born outside of Japan to 2nd generation Japanese parents.

### Additional Inclusion Criteria:

At screening, vital signs (systolic and diastolic blood pressure and pulse rate) will be assessed in the sitting position after the participant has rested for at least three minutes, and again (when required) after three minutes in the standing position. If sitting systolic blood pressure, diastolic blood pressure, or pulse rate are out-of-range at screening, two additional readings of each can be obtained, so that up to three consecutive assessments are made, with the participant seated quietly for approximately five minutes preceding each repeat assessment. The last reading must be within the ranges listed below. Oral body temperature should not be repeated if our of range. Sitting vital signs should be within the following ranges:
   All cohorts: oral body temperature between 35.0-37.5° C.; pulse rate, 40-90 bpm.
   Cohorts 1-8: systolic blood pressure, 110-139 mm Hg; diastolic blood pressure, 70-89 mm Hg.
   Cohort 9-10: systolic blood pressure, 139-159 mm Hg; diastolic blood pressure, 75-95 mm Hg.

### Key Exclusion Criteria:

Any surgical or medical condition which might significantly alter the distribution, metabolism, or excretion of drugs, or which may jeopardize the participant's participation in the study.
Known history or current clinically significant arrhythmias
Women of child-bearing potential are excluded from this study.

The rationale for the starting dose and the maximum dose is in accordance with the FDA and EMA guidelines: 1) Estimating the Maximum Safe Starting Dose in Initial Clinical Trials for Therapeutics in Adult Healthy Volunteers (CDER 2005); 2) Guideline on Strategies to Identify and Mitigate Risks for First-In-Human and Early Clinical Trials with Investigational Medicinal Products (IMP) EMA (CHMP 2018). The FIH starting dose of the described antibody is determined and selected based on toxicology findings and in consideration of the minimum anticipated biological effect level (MABEL).

### Estimation of Maximum Recommended Starting Dose (MRSD) Based on the NOAEL

Step 1: Determine NOAEL. Rat (Wistar Han): 50 mg/kg/week IV and 50 mg/kg/week SC for male and female rats in a 13-week GLP repeat dose toxicology study. Monkey (cynomolgus): 50 mg/kg/week IV and 50 mg/kg/week SC for male and female monkeys in a 13-week GLP repeat dose toxicology study.
Step 2: Calculation of human equivalent dose. As is appropriate for a monoclonal antibody with molecular weight >100 kDa, the calculation of the human equivalent dose (HED) is based on body weight (mg/kg) scaling, therefore, HED from the NOAEL is 50 mg/kg.
Step 3: Most appropriate species selection. Based on the GLP 13-week rat and monkey toxicology studies there was no difference in toxicity, as 50 mg/kg/week after IV and SC administration was the NOAEL for both species.
Step 4: Application of safety factor. The HED dose of 50 mg/kg with the application of a safety factor of 10 results in the maximum recommended safe starting dose (MRSD) of 50 mg/kg/10=5 mg/kg, which is equivalent to a SC injection of 350 mg (70 kg person).

### Estimation of PAD and MABEL

Step 5: Consideration of the pharmacologically active dose. The lowest dose of the antibody that led to the sustained intended pharmacological activity was 0.3 mg/kg SC in rats. Correcting for a 3-fold greater potency in humans, this yields a human dose of approximately 7 mg (based on human 70 kg body weight).

The MABEL criteria is defined as a dose that results in a minimal possible biological effect, in this case, it is defined as systolic blood pressure (SBP) lowering of <1%. For a participant with a baseline SBP of 140 mmHg, this corresponds to absolute blood pressure (BP) lowering of 1 to 2 mmHg.

Based on rat and monkey data, the reduction in SBP in human may be 0.6% and 0.9%, respectively, after 1 mg SC (based on rat data) and after 10 mg SC (based on monkey data). The proposed human starting dose is therefore set at 1 mg SC based on rat data, which is a more conservative estimate for the starting dose.

The starting dose of 1 mg (˜0.01 mg/kg) is 1/350 of the MRSD of 350 mg. Exposure multiples comparing rat and monkey NOAEL exposures to human predicted exposure for 1 mg SC starting dose are greater than 1000-fold based on Cmax and AUC (0-168 h).

Dosing will be conducted in a step-wise approach for each cohort starting with the dosing of the two sentinel participants. For the first two participants in each dose cohort, one participant receives the described antibody and one participant receives placebo in a randomized and blinded manner. These sentinel participants (following same assessment schedule as remainder of the cohort) will be observed and monitored up to and including Day 10 to allow for detection of any events or reactions before allowing the remainder of the cohort to be dosed.

Eight participants per cohort will be randomized to receive a single dose of the described antibody or a matching placebo in a 6:2 ratio. A total of up to approximately 80 participants will be enrolled in the planned cohorts, excluding replacements. Planned dose levels are described in Table 3. For the two higher dose cohorts (450 mg and 600 mg), healthy subjects with elevated blood pressure will explore potential pharmacodynamic effects of the described antibody on blood pressure.

TABLE 3
Dose levels per cohort

| | Proposed | |
|---|---|---|
| Cohort | Dose | Participants |
| 1a | 1 mg | 2 (1 active; 1 placebo) |
| 1b | 1 mg | 6 (5 active; 1 placebo) |
| 2a | 3 mg | 2 (1 active; 1 placebo) |
| 2b | 3 mg | 6 (5 active; 1 placebo) |
| 3a | 10 mg | 2 (1 active; 1 placebo) |
| 3b | 10 mg | 6 (5 active; 1 placebo) |
| 4a | 30 mg | 2 (1 active; 1 placebo) |
| 4b | 30 mg | 6 (5 active; 1 placebo) |
| 5a | 60 mg | 2 (1 active; 1 placebo) |
| 5b | 60 mg | 6 (5 active; 1 placebo) |
| 6a | 120 mg | 2 (1 active; 1 placebo) |
| 6b | 120 mg | 6 (5 active; 1 placebo) |
| 7a | 240 mg | 2 (1 active; 1 placebo) |
| 7b | 240 mg | 6 (5 active; 1 placebo) |
| 8 | 240 mg | 8 (6 active; 2 placebo) |
| 9a | 450 mg | 2 (1 active; 1 placebo) |
| 9b | 450 mg | 6 (5 active; 1 placebo) |
| 10a | 600 mg | 2 (1 active; 1 placebo) |
| 10b | 600 mg | 6 (5 active; 1 placebo) |

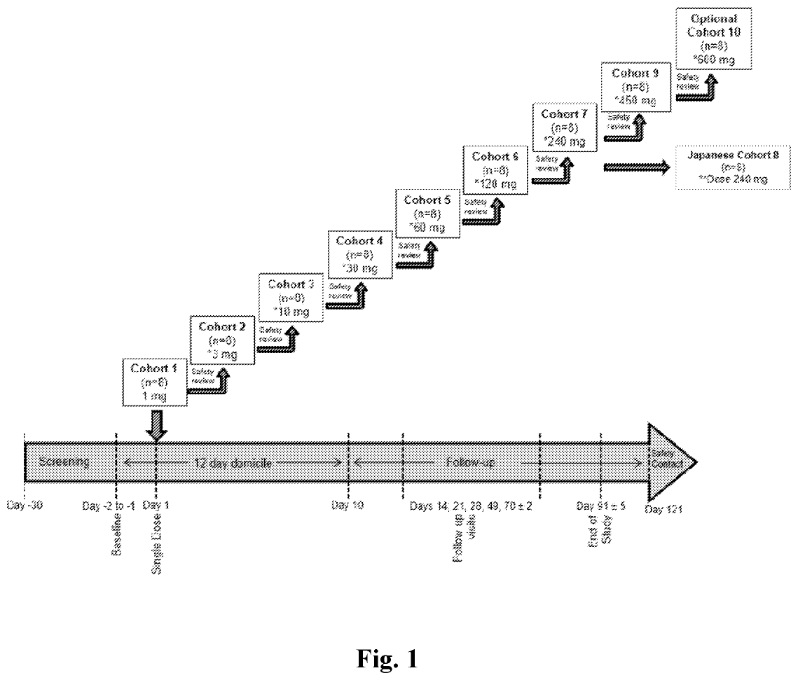

The study is comprised of a screening period up to 28 days, a baseline evaluation (Day-2 to Day-1), a domicile period (Day-2 through Day 10, that is, domiciling up to Day 10, which may be adjusted in accordance with medical advice), a single SC dose of the antibody described herein or placebo on Day 1, and the End of Study (EOS) visit will occur on Day 91. Participants will also return to the site on Day 29 and Day 92 to return the blood pressure devices after completion of 24 hour monitoring. Total number of follow-up visits after the domicile period is eight. A graphical representation of the overall study design is included as FIG. 1.

### Interim Analysis

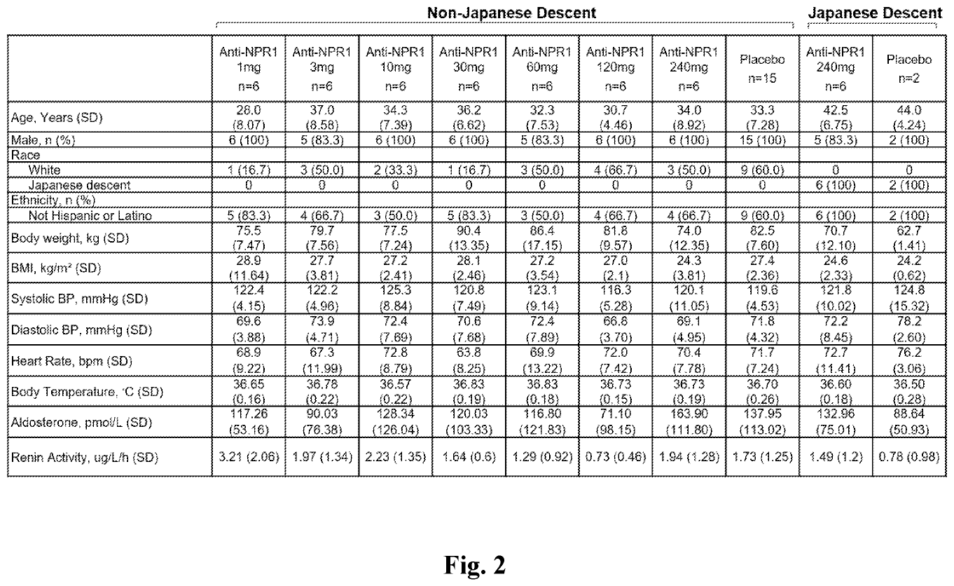

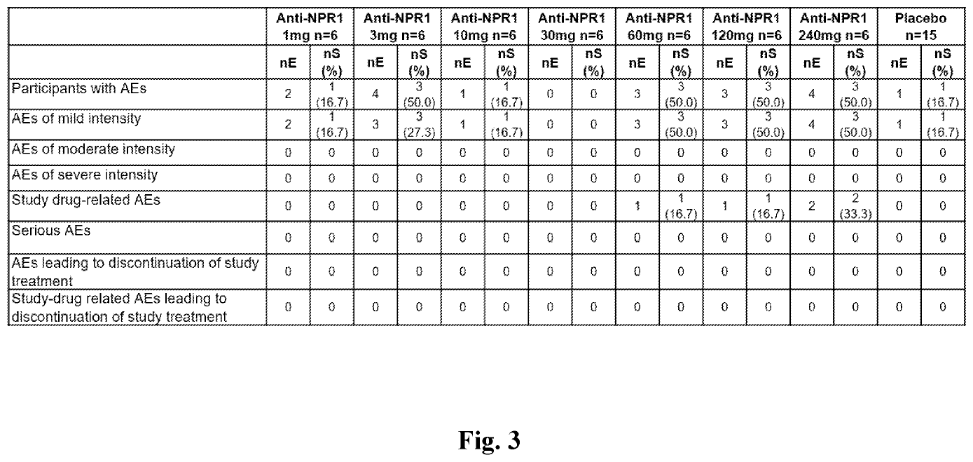

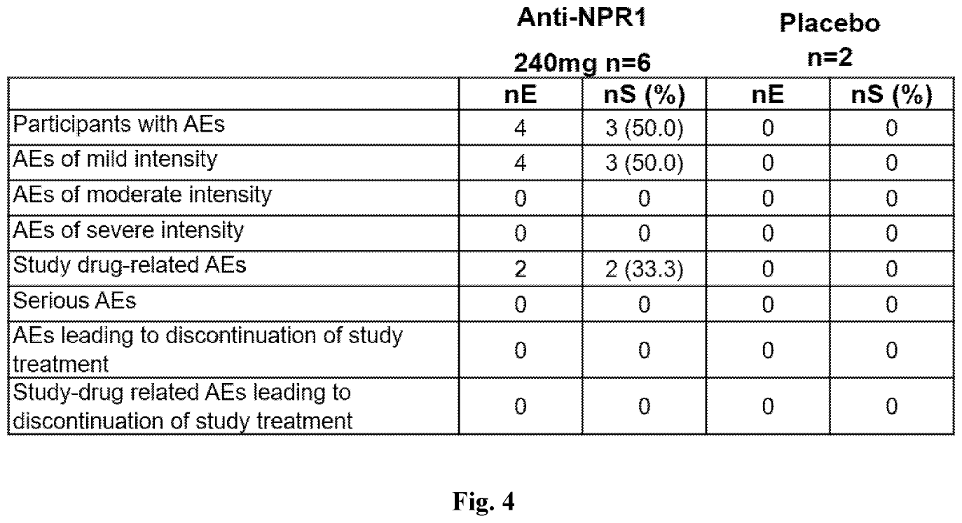

An interim analysis (IA) was performed for eight dose cohorts after a single subcutaneous dose of the anti-NPR1 antibody. The demographics and baseline characteristics of the information for participants analyzed in this IA are shown in FIG. 2. The anti-NPR1 antibody was generally considered safe and well tolerated after a single dose over the dose range from 1 mg to 240 mg. Results from the IA in participants of both non-Japanese descent; and Japanese descent are shown in FIG. 3 and FIG. 4, respectively. All adverse events (AEs) reported were mild in intensity and resolved spontaneously. There were no moderate or severe AEs reported.

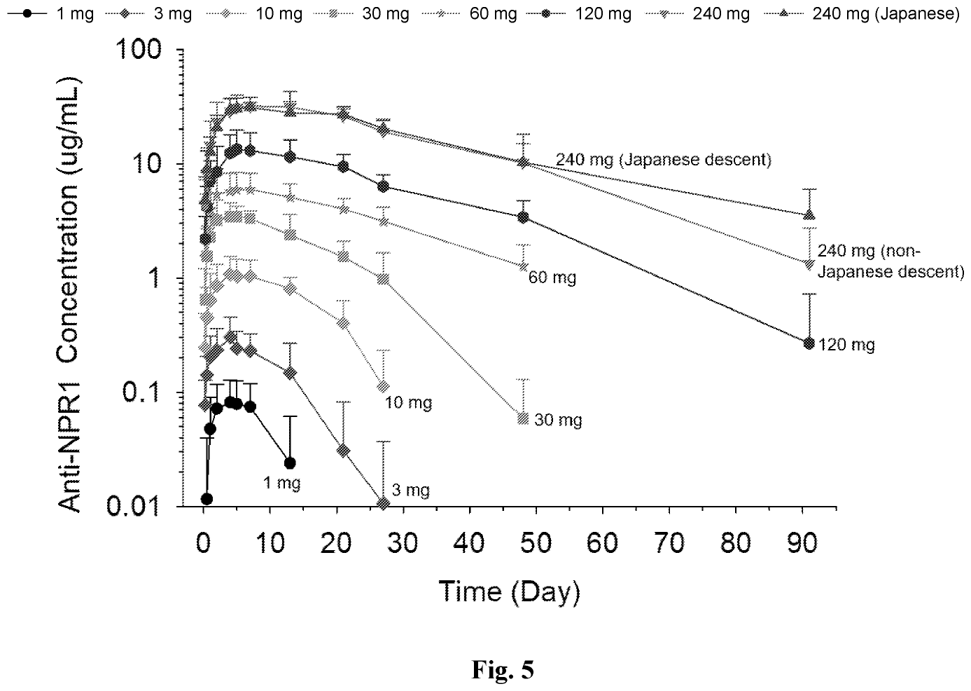

Serum concentrations of the anti-NPR1 antibody were measured. Pharmacokinetic (PK) profiles for the anti-NPR1 antibody after a single subcutaneous dose of 1 to 240 mg for the non-Japanese descent cohort and the Japanese descent 240 mg dose cohort are shown in FIG. 5. The Tmax was determined to be 4˜10 days and the half-life (T½) at 120 and 240 mg was determined to be 15-23 days.

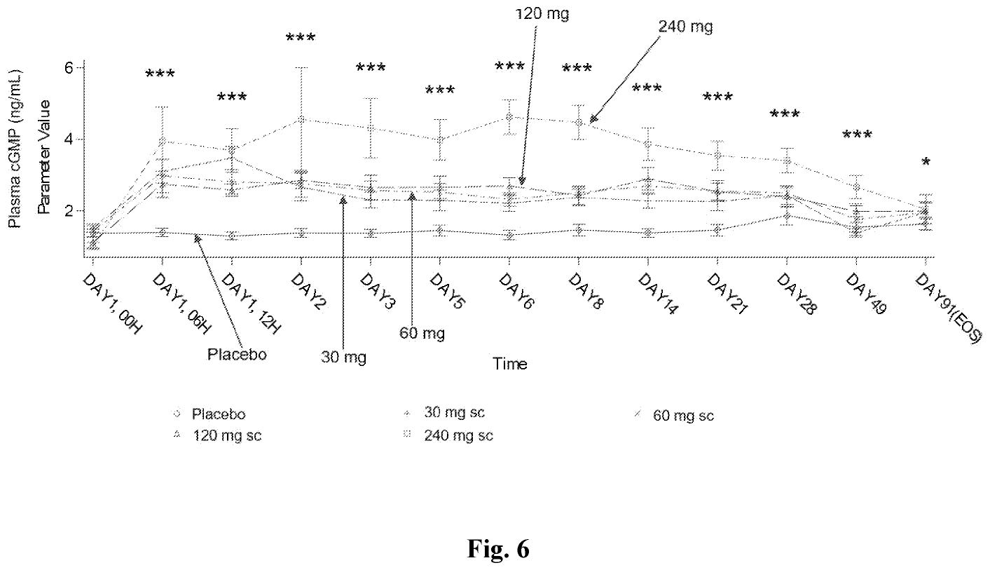

The anti-NPR1 antibody increased plasma levels of cGMP and reduced blood pressure at doses of 30 mg, 60 mg, 120 mg, and 240 mg. Maximal effects on cGMP and blood pressure were achieved on Days 2-3 after a single dose of greater than or equal to 30 mg. Interim analysis results for the non-Japanese descent cohorts over time after a single subcutaneous administration of the anti-NPR1 antibody at 30 mg, 60 mg, 120 mg, and 240 mg are shown in FIG. 6. For the 240 mg dose cohort: ***p<0.001; cGMP levels increased by 200-400% from Day 1 through Day 14 followed by progressive attenuation; *p<0.05; 50% elevation after 240 mg dose at Day 91 (EOS).

Blood pressure and heart rate were measured using three methods: 1) in-office measurements; 2) Ambulatory Blood Pressure Monitoring (ABPM) device; 3) other wearable device. Each visit day of ABPM is a 24-hour period; blood pressure and heart rate data was collected every 15 minutes by the ABPM device. The Baseline day of ABPM is defined as 24 hours, counting back from the last assessment which must be before 9 am of Day 1 and before dosing of the anti-NPR1 antibody on Day 1. The 24-hour baseline ABPM data is calculated as the average of all blood pressure or heart rate data points over the specified time period. The 24-hour average blood pressure or heart rate for other days (2, 5, 8, 28, and 91) were calculated in the same manner as the baseline.

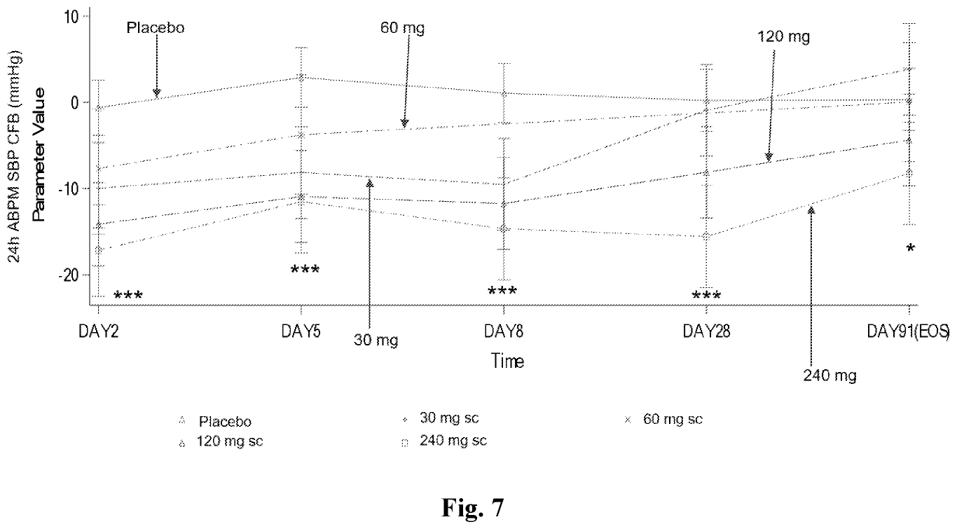

24-hour ADPM SBP change from Baseline (mmHg) is shown in FIG. 7. BP/HR data were collected every 15 min from the ABPM device. Each data point on each day is the average of approximately 96 of BP or HR measurements. ABPM BP data for the 60 mg cohort is missing for Day 8 and Day 28. For the 240 mg dose cohort: ***p<0.001; SBP significantly reduced by 14-16 mmHg during Day 2˜28 (when ABPM was implemented). *p<0.05; significantly reduction in SBP (8.4 mmHg) after 240 mg dose at Day 91 (EOS). SBP was reduced by up to 16 mmHg at the highest dose tested (240 mg), which was sustained for up to 4 weeks.

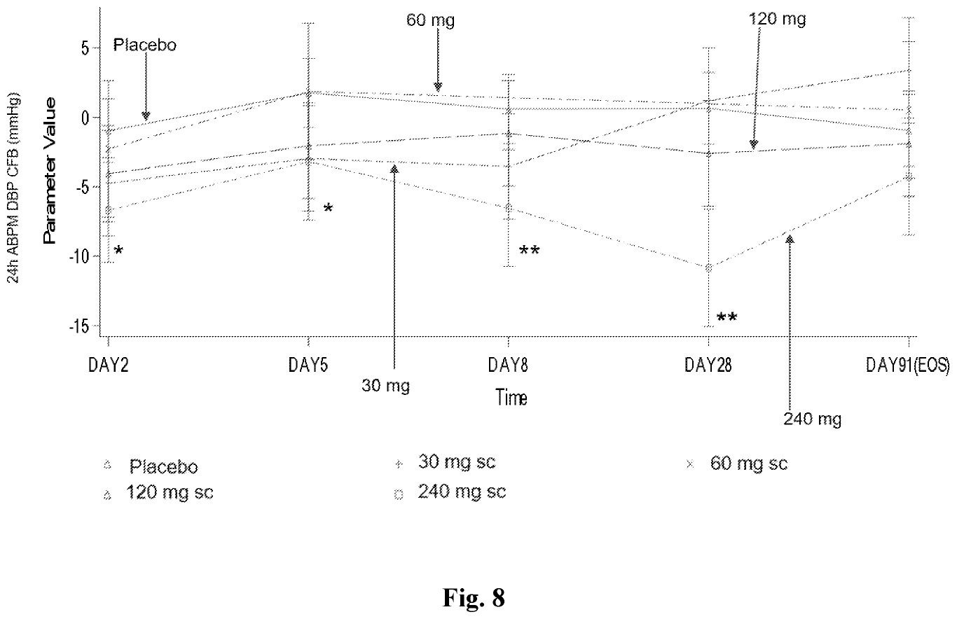

24-hour ADPM DBP change from Baseline (mmHg) is shown in FIG. 8. ABPM BP data for the 60 mg cohort is missing for Day 8 and Day 28. For the 240 mg dose cohort, DBP was significantly reduced by 6-12 mmHg (*p<0.05, **p<0.01) during Day 2˜28 (when ABPM was implemented).

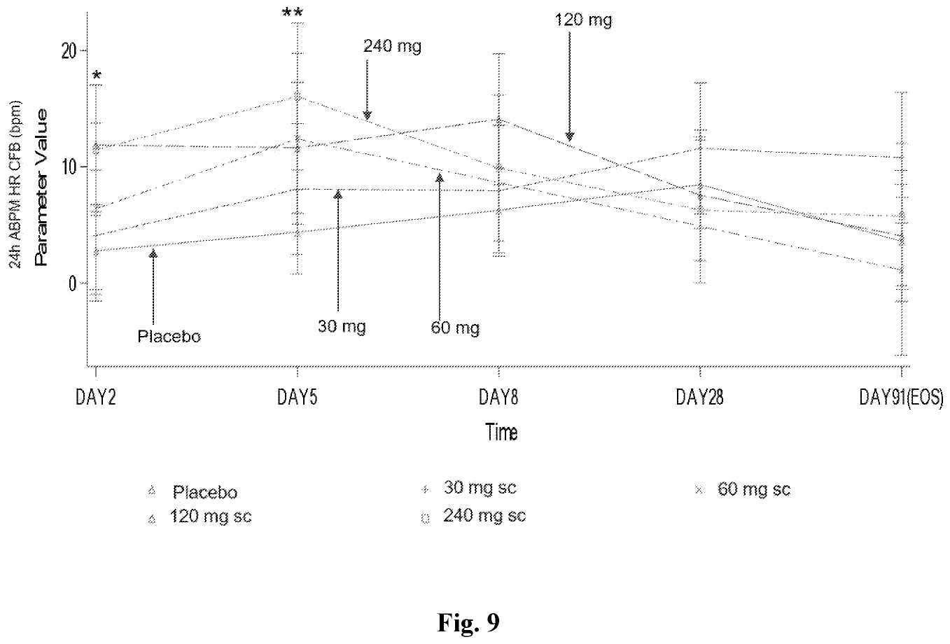

24-hour ABPM heart rate (HR) change from Baseline (bpm) is shown in FIG. 9. For the 240 mg dose cohort: *p<0.05; HR significantly increased (9 bpm) on Day 2; **p<0.01; HR significantly increased (12 bpm) on Day 5; and no significant increase on Day 8, 28 and 91 (EOS). A statistically significant increase in HR on Day 2 and/or Day 5 was observed at doses of ≥120 mg.

### Example 2: A Safety and Tolerability Study in Heart Failure Participants with Reduced Ejection Fraction (HFrEF) or Mildly Reduced Ejection Fraction (HFmrEF)

Below is described a randomized, participant and investigator blinded, sponsor open-label placebo-controlled single and multiple dose study to investigate the safety and tolerability of a single subcutaneous (SC) dose of an anti-NPR1 antibody in heart failure participants with reduced ejection fraction (HFrEF) or mildly reduced ejection fraction (HFmrEF). The purpose of these studies is to evaluate the safety and tolerability of a single subcutaneous dose or multiple subcutaneous doses of an anti-NPR1 antibody in adult participants with chronic stable HFrEF; and to evaluate the pharmacokinetics of such an antibody in the same.

The study population will be comprised of approximately 18-24 men and women, aged 18-75 years old or 18-80 years old with HFrEF or mildly reduced ejection fraction (HFmrEF) defined as New York Heart Association (NYHA) class II-III and left ventricular ejection fraction (LVEF) less than or equal to 45% or less than or equal to 50%. The NYHA functional class definitions are described in Table 4.

TABLE 4
New York Heart Association (NYHA) functional class

| | |
|---|---|
| I | No limitation of physical activity. Ordinary physical activity does not cause undue fatigue, |
| | palpitation, dyspnea (shortness of breath) |
| II | Slight limitation of physical activity. Comfortable at rest. Ordinary physical activity |
| | results in fatigue, palpitation, dyspnea (shortness of breath) |
| III | Marked limitation of physical activity. Comfortable at rest. Less than ordinary activity |
| | causes fatigue, palpitation, or dyspnea |
| IV | Unable to carry on any physical activity without discomfort. Symptoms of heart failure at |
| | rest. If any physical activity is undertaken, discomfort increases |

LVEF will be assessed locally by echocardiography if no prior LVEF assessment was performed within six months prior to screening.

A screening period of up to 28 days will be used to assess participant eligibility. Once selected, approximately 18 eligible participants (excluding replacements) will be randomized in a 2:1 ratio to receive a single dose of either the described antibody or a placebo after administration to a sentinel cohort of two participants. The participants will be stratified by LVEF status (LVEF less than or equal to 35%; vs. LVEF greater than 35% and less than or equal to 45%. Approximately eight participants will be included with LVEF of less than or equal to 35%. One of the two sentinel patients will be administered the anti-NPR1 antibody; and one of the two sentinel patients will receive the placebo. The two sentinel patients will not be chosen based on LVEF status.

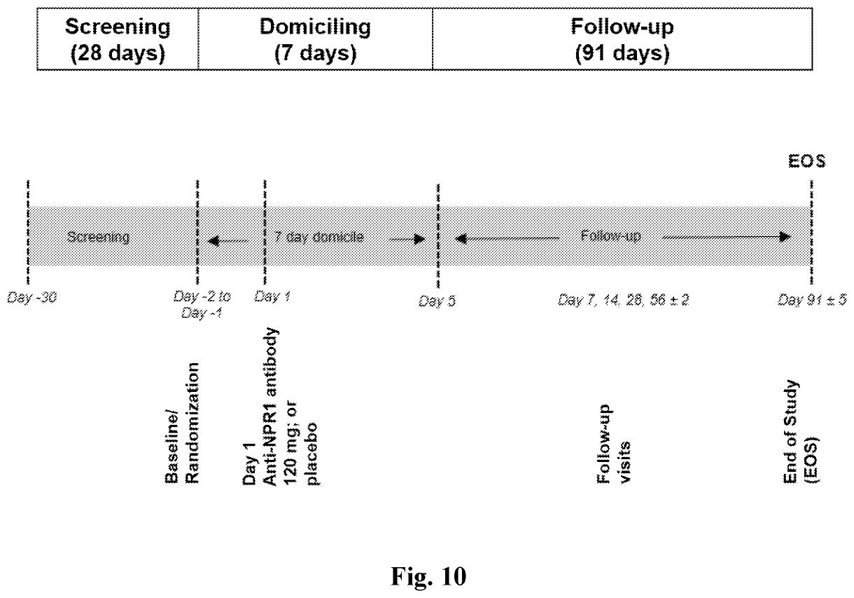

Chronic stable HFrEF participants meeting eligibility requirements will be admitted to baseline evaluations on Day-2. Baseline evaluations will be completed prior to randomization. For Cohort 1, after the initial domiciling baseline evaluation (Day-2 to Day-1), a single 120 mg subcutaneous (s.c.) dose of the antibody or the placebo will be administered on Day 1 (onsite). The participants will continue to be domiciled for a five day period (for a total of 7 days domiciled from Day-2 through Day 5). Following the domiciled period, participants will be followed for thirteen weeks post-dosing for safety, tolerability, and PK until the end-of-study (EOS) visit on Day 91. Participants will be evaluated during approximately five follow-up visits during the 13 week post-dosing period ending with the end of study (EOS) visit. Such follow-up visits will occur approximately on days 7, 14, 28, 56, and 91 (EOS). A graphical representation of the overall study design with respect to Cohort 1 is presented in FIG. 10.

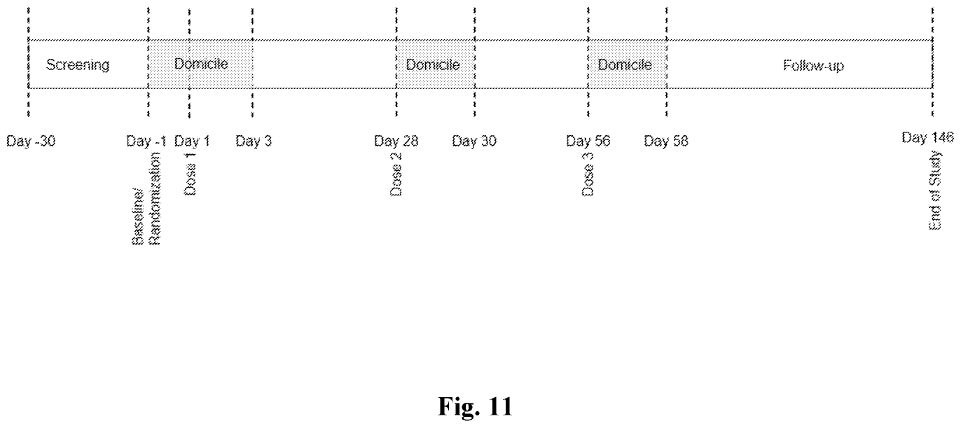

For Cohort 2, after the initial domiciled baseline evaluation on Day 1, a 120 mg subcutaneous (s.c.) dose of the antibody or placebo will be administered on Day 1 (onsite). Participants will then be domiciled for 3 days (a total of 4 days domicile from Day-1 through Day 3). On Day 28, participants will receive a second dose of 120 mg of the antibody or placebo and be re-domiciled for 3 days (Day 28 through Day 30). On Day 56, participants will receive a third dose of 240 mg of the antibody or placebo and be re-domiciled for 3 days (Day 56 through Day 58). Follow-up visits (safety, tolerability, and PK) after each dose will occur out to 21 weeks post dose with the end of study (EOS) visit on Day 146. A graphical representation of the overall study design with respect to Cohort 2 is presented in FIG. 11.

The patients in this study will have chronic HFrEF and will be currently taking a background therapy that may include ACEi/ARBs, beta-blockers, and/or diuretics. Approximately eight of the enrolled participants will have LVEF of less than or equal to 35% documented at screening in order to ensure an appropriate patient population representation.

Certain participants will be administered a single 120 mg subcutaneous dose of the described antibody or a placebo. The antibody or placebo will be administered in a single injection. Such patients may be considered to be in Cohort 1, and be on an ACEi/ARB background therapy.

Certain participants will be administered three subcutaneous doses of the described antibody or a placebo: 120 mg at Day 1, 120 mg at Day 28, and 240 mg at Day 56. The antibody or placebo will be administered in a single injection. Such patients may be considered to be in Cohort 2, and be on a sacubratril/valsartan background therapy.

### Key Inclusion Criteria:

Men or women aged 18 to 75 years
NYHA functional class II-III
LVEF≤45% or ≤50% documented at screening
Systolic blood pressure (SBP) 110-180 mmHg (inclusive; seated) and resting heart rate of ≤90 beats per minute; or Systolic blood pressure (SBP) 110-160 mmHg (Cohort 1) or SBP 105-160 mmHg (Cohort 2), and heart rate 50-90 beats per minute inclusive, at screening
On stable therapies of ACE inhibitor or an ARB for at least four weeks prior to the screening visit; and a beta blocker for four weeks prior to the screening visit

### Key Exclusion Criteria:

Acute heart failure (e.g., acute decompensated heart failure within three months prior to screening), acute coronary syndrome, stroke, transient ischemic attack; cardiac, carotid, or other major cardiovascular surgery; percutaneous coronary intervention (PCI), or carotid angioplasty within the six months prior to screening
Hemodynamically significant mitral and/or aortic valve disease, except mitral regurgitation secondary to left ventricle (LV) dilation at screening
Implantation of a cardiac resynchronization therapy (CRT) device within three months prior to screening or intent to implant a CRT during the study period History of severe pulmonary disease (e.g., COPD) requiring chronic supplemental oxygen therapy or pulmonary hypertension requiring pharmacology treatment at screening
Estimated glomerular filtration rate (eGFR) of less than 45 mL/min/1.73 m² at screening
treatment with sacubitril/valsartan currently or within two weeks from screening
treatment with nesiritide currently or within two weeks from screening
BMI of greater than 35 kg/m²; or BMI of greater than 40 kg/m²
For possible cohorts:
   i. Cohort 1 only: Treatment with sacubitril/valsartan currently or within 4 weeks from screening
   ii. Cohort 2 only: Treatment with ACEi or ARB currently or within 4 weeks from screening. Valsartan as part of sacubitril/valsartan is not an exclusion.

### Assessments:

Efficacy assessments
i. Pulse pressure
ii. Clinical congestion score
iii. NYHA class
Pharmacodynamic assessments:
i. Blood pressure
ii. Plasma and urinary cGMP
Pharmacokinetic assessments:
i. Assessments of antibody PK in blood

The clinical congestion score can range from 0 to 16 and will be calculated as described below in Table 5.

TABLE 5
Clinical congestion score

| | Paroxysmal | | | Jugular | | Rales on |
|---|---|---|---|---|---|---|
| | noctural | Dyspnea | | venous | Peripheral | lung |
| | dyspnea | on effort | Orthopnea | distension | edema | auscultation |
| Absent | 0 | 0 | 0 | 0 | 0 | 0 |
| Present | 1 | If present: | If present: | If present: | If present: | If present: |
| | | Mild: 1 | 1 pillow | <6 cm: 1 | Slight/feet | Less than |
| | | | (10 cm | | and ankles: 1 | ⅓: 1 |
| | | | elevation): 1 | | | |
| | | Moderate: 2 | 2 pillows | 6 to 9 cm: 2 | Moderate/to | ⅓ to ⅔: 2 |
| | | | (20 cm | | lower legs or | |
| | | | elevation): 2 | | thighs: 2 | |
| | | Severe: 3 | >2 pillows | >9 cm: 3 | Marked/to | >⅔: 3 |
| | | | (>20 cm | | sacrum: 3 | |
| | | | elevation): 3 | | | |

Pulse pressure (PP) is a measure of arterial stiffness and will be calculated as PP=systolic blood pressure (SBP)−diastolic blood pressure (DBP). PP will be calculated from the blood pressure measurements performed as part of the vital signs assessments throughout the study.

### Example 3: An Efficacy, Safety, Tolerability and Dose Finding Study of an Anti-NPR-1 Antibody in Resistant Hypertension Patients

Below is described a planned multi-center, randomized, double-blind, parallel-group, 20-week dose-finding study to evaluate efficacy, safety, and tolerability of an anti-NPR1 antibody in patients with resistant hypertension.

This study in patients with resistant hypertension (rHTN) will evaluate the efficacy, safety, and tolerability, of four doses of an anti-NPR1 antibody (30 mg, 60 mg, 120 mg and 240 mg) which will be administered once every 4 weeks (q4w) as subcutaneous (SC) injections, compared to placebo. Since all study participants will be patients with rHTN, study treatments will be given on top of maximally tolerated background antihypertensive therapy recommended by international guidelines for treatment of HTN (i.e., a thiazide or a thiazide-like diuretic, an angiotensin converting enzyme inhibitor (ACEI) or an angiotensin receptor blocker (ARB), and a long-acting dihydropyridine calcium channel blocker (CCB)). Approximately 170 participants will be randomized to one of the following five treatment arms targeting a final ratio of 1:1:1:1:1 at the end of the trial:
Placebo SC every 4 weeks for 12 weeks
Anti-NPR1 antibody at 30 mg SC every 4 weeks for 12 weeks (dose level 1)
Anti-NPR1 antibody at 60 mg SC every 4 weeks for 12 weeks (dose level 2)
Anti-NPR1 antibody at 120 mg SC every 4 weeks for 12 weeks (dose level 3)
Anti-NPR1 antibody at 120 mg SC at the Randomization visit followed by 240 mg SC at Week 4 and Week 8 (dose level 4)

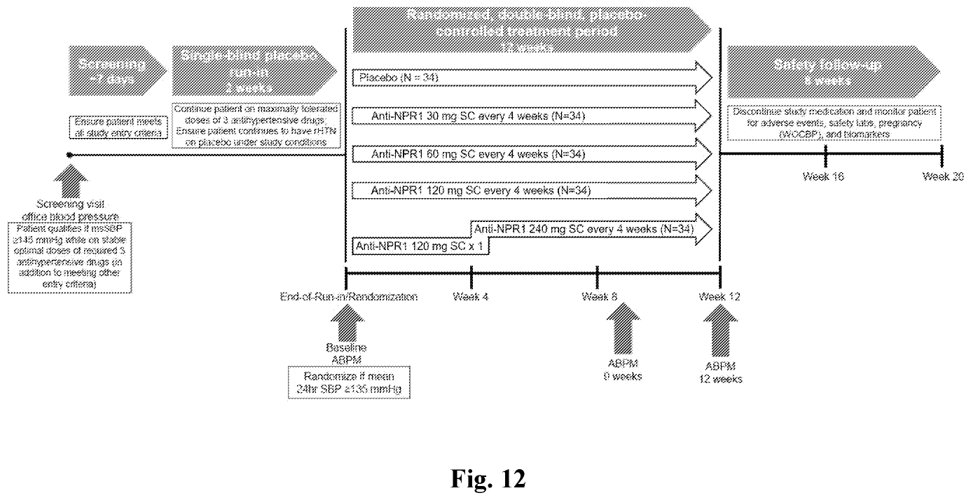

A graphical representation of the overall study design is included as FIG. 12.

A second stage of this protocol will be added to the study to evaluate the efficacy and safety of the anti-NPR1 antibody against an active comparator after dose response has been established against placebo. Specific doses of anti-NPR1 antibody based on the dose-finding stage (Stage 1) of the study will be compared in a double-blind manner to spironolactone 50 mg once daily for 12 weeks in a second stage of this study.

This planned study includes four periods:
A screening period (approximately 7 days)
A single-blind placebo run-in period lasting approximately 2 weeks
A 12-week double-blind, placebo-controlled, parallel-group treatment period.
An 8-week safety follow-up period

### Key Inclusion Criteria

Male and female participants who are ≥18 years old.
Signed informed consent prior to participation in the study.
Apparent rHTN at screening (Visit 1) defined as uncontrolled BP with an office msSBP≥145 mmHg despite treatment with stable (i.e., unchanged for >4 weeks), maximally tolerated doses of three antihypertensive drugs of different classes, specifically an ACEI/ARB, a long-acting dihydropyridine CCB, and a thiazide or thiazide-like diuretic.
Mean 24 hr SBP≥135 mmHg (measured by ABPM) at the end-of Run-in-Visit (Visit 30) on treatment with maximally tolerated doses of an ACEI/ARB, a long-acting dihydropyridine CCB, and a thiazide or thiazide-like diuretic.

### Key Exclusion Criteria

Office msSBP<140 mmHg at Visit 20 or Visit 30 or office msSBP≥180 mmHg or office msDBP>110 mmHg at the end-of-run-in visit (Visit 30) OR 24 h mean SBP≥170 mmHg or 24 h mean DBP>105 mmHg measured by ABPM at the end of the run the Run-in (Visit 30).
Known history of secondary hypertension (moderate-to-severe obstructive sleep apnea without receiving CPAP therapy (either face mask or nasal device), renovascular hypertension, primary aldosteronism, pheochromocytoma, Cushing syndrome, aortic coarctation or other cause of secondary hypertension).
Estimated GFR<30 mL/min/1.73 m2 using CKD-Epi equation at screening (Visit 1) or at end-of-run-in visit (Visit 30).
Serum potassium >5.0 mmol/L (or equivalent plasma potassium value) at screening or end-of-run-in visit (Visit 30).
Current therapy with a mineralocorticoid receptor antagonist (MRA) or received an MRA within the 4 weeks prior to screening.
Clinically significant cardiac arrhythmias (e.g., ventricular tachycardia), high-grade AV block (e.g., Mobitz type II and third-degree AV block in absence of a pacemaker) within 6 months of screening according to investigator's judgement.
Received any antihypertensive medication other than the CCB, ACEI/ARB, and thiazide/thiazide-like diuretic components of the triple background antihypertensive therapy, including sacubitril/valsartan within the 4 weeks before screening (Visit 1). Participants receiving beta blockers and prostate-specific alpha blockers (e.g., tamsulosin) are allowed in the study only if those medications are being used non-hypertension indications and benign prostatic hypertrophy, respectively.

Objectives and endpoints for the described study may be found in Table 6.

TABLE 6
Objectives and Endpoints

| Objective(s) | Endpoint(s) |
|---|---|
| Primary objective(s) | Endpoint(s) for primary objective(s) |
| To evaluate the efficacy and dose-response | Change from baseline in mean 24 hr SBP at Week |
| relationship of the anti-NPR1 antibody 30 mg SC | 12 |
| q4w, 60 mg SC q4w, 120 mg SC q4w, and 240 mg | |
| SC q4w compared to placebo in reducing the mean | |
| 24 hr ambulatory systolic blood pressure (mean 24 hr | |
| SBP) from baseline at Week 12. | |
| Secondary objective(s) | Endpoint(s) for secondary objective(s) |
| To evaluate the treatment effect of the highest | Change from baseline in mean 24 hr SBP at Week |
| anti-NPR1 antibody dose versus placebo in | 12 |
| reducing the mean 24 hr SBP from baseline to Week | |
| 12. | |
| To characterize the dose-response relationship of | Nocturnal SBP dipping expressed as (nighttime |
| the anti-NPR1 antibody 30 mg SC q4w, 60 mg SC | mean SBP/daytime mean SBP) at Week 12 |
| q4w, 120 mg SC q4w, and 240 mg SC q4w | |
| compared to placebo in the nocturnal SBP dipping | |
| at Week 12 | |
| To evaluate the proportions of participants | The proportions of participants achieving blood |
| achieving ambulatory BP control (i.e., mean 24 hr | pressure control defined as mean 24 hr SBP <130 |
| SBP <130 mmHg and mean 24 hr DBP <80 | mmHg and mean 24 hr DBP <80 mmHg at Week 12 |
| mmHg) with respect to the dose-response | |
| relationship of the four anti-NPR1 antibody dose | |
| level groups compared to placebo at week 12. | |
| To evaluate the safety and tolerability of the anti- | Adverse events, safety laboratory parameters, and |
| NPR1 antibody regimens over 12 weeks of | vital signs through end of treatment/study |
| treatment and over the 20-week study period | (EOT/EOS) |
| including safety follow-up. | |
| Additional objective(s) | Endpoint(s) for Additional objective(s) |
| To assess the proportions of participants | The proportions of participants achieving office |
| achieving office BP control based on office BP | BP control defined as office msSBP <135 mmHg |
| defined as mean sitting systolic BP (msSBP) <135 | and msDBP <80 mmHg at Week 12 |
| mmHg and mean sitting diastolic BP (msDBP) <80 | |
| mmHg for the anti-NPR1 antibody dose regimens | |
| compared to placebo at Week 12 | |
| To assess the proportions of participants | The proportions of participants achieving home |
| achieving home BP control based on home BP | BP control defined as home mwSBP <130 mmHg |
| defined as mean weekly systolic BP (mwSBP) <130 | and mwDBP <80 mmHg at Week 12 |
| mmHg and mean weekly diastolic BP (mwDBP) | |
| <80 mmHg for the anti-NPR1 antibody dose | |
| regimens compared to placebo at Week 12. | |
| To characterize the dose-response relationship of | Change from baseline in mean 24 hr DBP, office |
| the four anti-NPR1 antibody dosing regimens in | msSBP, office msDBP, home mwSBP, and home |
| reducing the mean 24 hr DBP, office msSBP, office | mwDBP at week 12 |
| msDBP, home mwSBP, and home mwDBP | |
| compared to placebo at Week 12. | |
| To compare the profile of ambulatory SBP, | AUC (Area Under Curve) of ambulatory SBP and |
| ambulatory DBP, mean sitting SBP (msSBP), mean | ambulatory DBP measurements; |
| sitting DBP (msDBP), home SBP (hSBP) and home | AUC of msSBP and msDBP measurements; |
| DBP (hDBP) during the dosing interval between the | AUC of home SBP and home DBP measurements. |
| four dose groups of the anti-NPR1 antibody and | |
| placebo at the specified timepoints | |
| To characterize the dose-response relationship of | Change from baseline in daytime and nighttime |
| the anti-NPR1 antibody regimens compared to | mean SBP and mean DBP at Week 12 |
| placebo in reducing daytime and nighttime mean | |
| SBP/mean DBP at Week 12 | |
| To explore the nocturnal DBP dipping response | Nocturnal DBP dipping expressed as (nighttime |
| of the four doses of the anti-NPR1 antibody | mean DBP/daytime mean DBP) at Week 12 |
| compared to placebo at Week 12 | |
| To explore the peak-to-trough ratio of the four | 24-hour ambulatory, daytime and nighttime |
| doses of the anti-NPR1 antibody and placebo using | SBP/DBP measurements at baseline, Week 9 and |
| ABPM at week 9 and Week 12 | Week 12 |
| To explore the pharmacokinetic (PK) properties | Population PK analysis including mathematic |
| of the anti-NPR1 antibody in rHTN participants | models and parameters |
| To evaluate immunogenicity of the anti-NPR1 | Detection of markers of immune response, |
| antibody by assessment of anti-drug antibodies | including anti-drug antibodies (ADAs) |
| To assess the effects of the treatment regimens on | Change from baseline at Week 12 in biomarkers |
| change from baseline in biomarkers related to | including but not limited to cGMP, aldosterone, |
| hypertension and/or cardiovascular disease | renin, NT-proBNP |
| To assess overall ambulatory SBP/DBP, | Ambulatory SBP/DBP, msSBP/msDBP, home |
| msSBP/msDBP, home SBP/DBP measurements in | SBP/DBP at scheduled assessment visits |
| terms of average over double-blind treatment period | |
| and safety follow-up period of the four anti-NPR1 | |
| antibody doses versus placebo | |

1. A method of treating a disorder or a disease associated with natriuretic peptide receptor 1 (NPR1) activity in a subject in need thereof, the method comprising administering to the subject about 10 mg to about 700 mg of an anti-NPR1 antibody or antigen binding fragment thereof.

2. The method of claim 1, wherein the disorder or disease associated with natriuretic peptide receptor activity is a cardiovascular disorder.

3. The method of claim 2, wherein the cardiovascular disorder is selected from: hypertension, peripheral vascular disease, heart failure, coronary artery disease (CAD), ischemic heart disease (IHD), mitral stenosis and regurgitation, angina, hypertrophic cardiomyopathy, diabetic cardiomyopathy, supraventricular and ventricular arrhythmias, cardiac dysrhythmia, atrial fibrillation (AF), new onset of atrial fibrillation, recurrent atrial fibrillation, cardiac fibrosis, atrial flutter, detrimental vascular remodeling, plaque stabilization, and myocardial infarction (MI).

4. The method of claim 2, wherein the cardiovascular disorder is heart failure.

5.-6. (canceled)

7. The method of claim 2, wherein the cardiovascular disorder is hypertension.

8.-9. (canceled)

10. The method of claim 1, wherein the anti-NPR1 antibody or antigen binding fragment thereof comprises three heavy chain complementarity determining regions (HCDR1, HCDR2, and HCDR3) and three light chain complementarity determining regions (LCDR1, LCDR2, and LCDR3) selected from:
(I) SEQ ID NO: 4 (HCDR1), SEQ ID NO: 5 (HCDR2), SEQ ID NO: 6 (HCDR3), SEQ ID NO: 17 (LCDR1), SEQ ID NO: 18 (LCDR2), and SEQ ID NO: 19 (LCDR3);
(II) SEQ ID NO: 7 (HCDR1), SEQ ID NO: 5 (HCDR2), SEQ ID NO: 6 (HCDR3), SEQ ID NO: 17 (LCDR1), SEQ ID NO: 18 (LCDR2), and SEQ ID NO: 19 (LCDR3);
(III) SEQ ID NO: 8 (HCDR1), SEQ ID NO: 9 (HCDR2), SEQ ID NO: 6 (HCDR3), SEQ ID NO: 20 (LCDR1), SEQ ID NO: 21 (LCDR2), and SEQ ID NO: 22 (LCDR3); or
(IV) SEQ ID NO: 10 (HCDR1), SEQ ID NO: 11 (HCDR2), SEQ ID NO: 12 (HCDR3), SEQ ID NO: 23 (LCDR1), SEQ ID NO: 21 (LCDR2), and SEQ ID NO: 19 (LCDR3).

11. The method of claim 1, wherein the anti-NPR1 antibody or antigen binding fragment thereof comprises:
(a) a heavy chain variable region comprising an amino acid sequence of SEQ ID NO: 13, and a light chain variable region comprising an amino acid sequence of SEQ ID NO: 24; or
(b) a heavy chain variable region comprising an amino acid sequence of SEQ ID NO: 28, and a light chain variable region comprising an amino acid sequence of SEQ ID NO: 24.

12. The method of claim 1, wherein the anti-NPR1 antibody or antigen binding fragment thereof comprises:
(a) a heavy chain comprising an amino acid sequence of SEQ ID NO: 15, and a light chain comprising an amino acid sequence of SEQ ID NO: 26; or
(b) a heavy chain comprising an amino acid sequence of SEQ ID NO: 30, and a light chain comprising an amino acid sequence of SEQ ID NO: 26.

13.-15. (canceled)

16. The method of claim 1, wherein the anti-NPR1 antibody or antigen binding fragment thereof is therapeutic.

17. The method of claim 1, wherein the antigen binding fragment is selected from the group consisting of a Fab, Fab′, F(ab′)2, Fv, and a single chain variable fragment (scFv).

18. The method of claim 1, wherein about 60 mg, about 120 mg, about 240 mg, about 450 mg, or about 600 mg of the anti-NPR1 antibody or antigen binding fragment thereof is administered to the subject.

19. The method of claim 18, wherein about 120 mg or about 240 mg of the anti-NPR1 antibody or antigen binding fragment thereof is administered to the subject.

20. The method of claim 1, wherein one or more additional therapeutically active agents are being administered to the subject.

21. The method of claim 20, wherein the one or more additional therapeutically active agents is selected from an ACE (angiotensin-converting-enzyme) inhibitor, an angiotensin receptor blocker (ARB), a neprilysin inhibitor, a beta blocker, a diuretic, a calcium channel blocker, a cardiac glycoside, a sodium-glucose co-transporter 2 inhibitor (SGLT2i), an angiotensin receptor-neprilysin inhibitor (ARNi), a corticosteroid, a leukotriene modifier, a bronchodilator, a beta-adrenoceptor antagonist, a carbonic anhydrase inhibitor, an alpha 2-adrenoceptor agonist, a parasympathomimetic, a prostaglandin analog, a rho kinase inhibitor, a cell therapy, a cardiac-specific myosin activator, and combinations thereof.

22. The method of claim 21, wherein the one or more additional therapeutically active agents is an ACE (angiotensin-converting-enzyme) inhibitor, an angiotensin receptor blocker (ARB), a beta blocker, a diuretic, or combinations thereof.

23. The method of claim 20, wherein the one or more additional therapeutically active agents is selected from enalapril, benazepril, captopril, fosinopril, lisinopril, moexipril, perindopril, quinapril, ramipril, trandolapril, valsartan, azilsartan, candesartan, eprosartan, irbesartan, losartan, olmesartan, telmisartan, sacubitril, bisoprolol, carvedilol, propanolol, metoprolol, metoprolol tartrate, metoprolol succinate, thiazide diuretics, loop diuretics, potassium-sparing diuretics, amlodipine, clevidipine, diltiazem, felodipine, isradipine, nicardipine, nifedipine, nisoldipine, verapamil, a digitalis glycoside, canagliflozin, dapagliflozin, empagliflozin, sotagliflozin, ertugliflozin, chlorothiazide, chlorthalidone, hydrochlorothiazide, indapamide, metolazone, bumetanide, ethacrynic acid, furosemide, torsemide, amiloride, eplerenone, finerone, spironolactone, triamterene, digoxin, fluticasone, budesonide, mometasone, beclomethasone, ciclesonide, fluticasone furoate, prednisone, methylprednisolone, montelukast, zafirlukast, zileuton, a long-acting beta agonist, a short-acting beta agonist, theophylline, ipratropium, salmeterol, formoterol, albuterol, levalbuterol, timolol, levobunolol, metipranolol, carteolol, betaxolol, acetazolamide, dorzolamide, brinzolamide, methazolamide, brimonidine, apraclonidine, a cholinomimetic, latanoprost, latanoprostene bunod, travoprost, bimatoprost, tafluprost, omecamtiv mecarbil, allogeneic cell therapy rexlemestrocel-L (REVASCOR®), CardiAMP™ cell therapy, netarsudil, ripasudil, and combinations thereof.

24. The method of claim 1, wherein the anti-NPR1 antibody or antigen binding fragment thereof is subcutaneously administered to the subject.

25. The method of claim 1, wherein the anti-NPR1 antibody or antigen binding fragment thereof is administered about once every week, once every 2 weeks, about once every 3 weeks, about once every 4 weeks, about once every 5 weeks, about once every 6 weeks, about once every 7 weeks, about once every 8 weeks, about once every 9 weeks, about once every 10 weeks, about once every 11 weeks, or about once every 12 weeks.

26. The method of claim 25, wherein the anti-NPR1 antibody or antigen binding fragment thereof is administered about once every 4 weeks.

27.-52. (canceled)
